SEQUENCE LISTING (206 sequences)

SEQ ID NO: 1 - DNA, 20 bp, Artificial
  Primer
cggccctaagtgatatgttg

SEQ ID NO: 2 - DNA, 22 bp, Artificial
  Primer
gaagagtgtgagggaaagactg

SEQ ID NO: 3 - DNA, 21 bp, Artificial
  Primer
aatgacaaattgagggagacg

SEQ ID NO: 4 - DNA, 22 bp, Artificial
  Primer
agagcccagtatcaattcttcc

SEQ ID NO: 5 - DNA, 20 bp, Artificial
  Primer
tcttgctgaacaaaggttcc

SEQ ID NO: 6 - DNA, 22 bp, Artificial
  Primer
tcccaggtatttgaagtgtttc

SEQ ID NO: 7 - DNA, 20 bp, Artificial
  Primer
taggcttctttccctccatc

SEQ ID NO: 8 - DNA, 20 bp, Artificial
  Primer
atgggtttgcaaagagttgg

SEQ ID NO: 9 - DNA, 23 bp, Artificial
  Primer
cattttcccctcaaataagacaa

SEQ ID NO: 10 - DNA, 23 bp, Artificial
  Primer
tctctttgatggaaaagaggaaa

SEQ ID NO: 11 - DNA, 24 bp, Artificial
  Primer
aaactttgtgctgttgggtgtatc

SEQ ID NO: 12 - DNA, 25 bp, Artificial
  Primer
ctcacctctgcctttctatctctct

SEQ ID NO: 13 - DNA, 22 bp, Artificial
  Primer
tggttcttctgtgatgagacag

SEQ ID NO: 14 - DNA, 20 bp, Artificial
  Primer
gcattggtcacgttaaatca

SEQ ID NO: 15 - DNA, 24 bp, Artificial
  Primer
ccaactggccagtcataataaatg

SEQ ID NO: 16 - DNA, 22 bp, Artificial
  Primer
gggccgcaaagggttggatgca

SEQ ID NO: 17 - DNA, 22 bp, Artificial
  Primer
ctggaaactggaaactagtggg

SEQ ID NO: 18 - DNA, 22 bp, Artificial
  Primer
gtgaggcattatcaagaagctg

SEQ ID NO: 19 - DNA, 24 bp, Artificial
  Primer
ttgtcagcaacttcttgtatcttt

SEQ ID NO: 20 - DNA, 24 bp, Artificial
  Primer
tgttttaagccacccaattatttg

SEQ ID NO: 21 - DNA, 21 bp, Artificial
  Primer
gagcaaggtgtttttccaatc

SEQ ID NO: 22 - DNA, 21 bp, Artificial
  Primer
cattctccaactgcttccttg

SEQ ID NO: 23 - DNA, 22 bp, Artificial
  Primer
ccatttctctgttggtaactgc

SEQ ID NO: 24 - DNA, 17 bp, Artificial
  Primer
ctctcacaggtggggtc

SEQ ID NO: 25 - DNA, 22 bp, Artificial
  Primer
agtcttctctgacagcagttgg

SEQ ID NO: 26 - DNA, 21 bp, Artificial
  Primer
ccaggtaccagagagaggaga

SEQ ID NO: 27 - DNA, 22 bp, Artificial
  Primer
ttacagaaagagtgtgtgtgcg

SEQ ID NO: 28 - DNA, 20 bp, Artificial
  Primer
ggctacagttcacaggttgc

SEQ ID NO: 29 - DNA, 22 bp, Artificial
  Primer
cattggtaggtgggttctttcc

SEQ ID NO: 30 - DNA, 23 bp, Artificial
  Primer
gcaacctaagtgtccatcaacag

SEQ ID NO: 31 - DNA, 22 bp, Artificial
  Primer
agggattggtttatgctctctc

SEQ ID NO: 32 - DNA, 20 bp, Artificial
  Primer
gttgcagagtcggacatgac

SEQ ID NO: 33 - DNA, 20 bp, Artificial
  Primer
gcaactaagacccaaccaac

SEQ ID NO: 34 - DNA, 22 bp, Artificial
  Primer
actgatgtgctcaggtatgacg

SEQ ID NO: 35 - DNA, 22 bp, Artificial
  Primer
tttcactgtcatctccctagca

SEQ ID NO: 36 - DNA, 22 bp, Artificial
  Primer
atgtattcatgcacaccacaca

SEQ ID NO: 37 - DNA, 22 bp, Artificial
  Primer
aaaatcccttcataacagtgcc

SEQ ID NO: 38 - DNA, 20 bp, Artificial
  Primer
catcgtgaattccagggttc

SEQ ID NO: 39 - DNA, 20 bp, Artificial
  Primer
aggaaggacaaggacattgc

SEQ ID NO: 40 - DNA, 21 bp, Artificial
  Primer
agagggtcaaaggcttaatgg

SEQ ID NO: 41 - DNA, 25 bp, Artificial
  Primer
gaaactcaacccaagacaactcaag

SEQ ID NO: 42 - DNA, 25 bp, Artificial
  Primer
atgactttattctccacctagcaga

SEQ ID NO: 43 - DNA, 20 bp, Artificial
  Primer
tgtgctggaggtgatagctg

SEQ ID NO: 44 - DNA, 22 bp, Artificial
  Primer
tgcaggaatatgagagctgaga

SEQ ID NO: 45 - DNA, 20 bp, Artificial
  Primer
agttggacctgccattgttc

SEQ ID NO: 46 - DNA, 19 bp, Artificial
  Primer
acttatgtgcgtgcgtgct

SEQ ID NO: 47 - DNA, 20 bp, Artificial
  Primer
gcaccagcagagaggacatt

SEQ ID NO: 48 - DNA, 20 bp, Artificial
  Primer
accggctattgtccatcttg

SEQ ID NO: 49 - DNA, 20 bp, Artificial
  Primer
cagacacgactaagcgacca

SEQ ID NO: 50 - DNA, 20 bp, Artificial
  Primer
cctacaataaagcacgggga

SEQ ID NO: 51 - DNA, 23 bp, Artificial
  Primer
gttcaggactggccctgctaaca

SEQ ID NO: 52 - DNA, 23 bp, Artificial
  Primer
cctccagcccactttctcttctc

SEQ ID NO: 53 - DNA, 24 bp, Artificial
  Primer
tctctctaatggagttggtttttg

SEQ ID NO: 54 - DNA, 24 bp, Artificial
  Primer
atatcccactgaggataagaattc

SEQ ID NO: 55 - DNA, 24 bp, Artificial
  Primer
gagtagaacacaactgaggacaca

SEQ ID NO: 56 - DNA, 20 bp, Artificial
  Primer
caatgctgtgggtactgagg

SEQ ID NO: 57 - DNA, 21 bp, Artificial
  Primer
gtaatgtagccttttgtgccg

SEQ ID NO: 58 - DNA, 22 bp, Artificial
  Primer
tcaccaacatgagatagtgtgc

SEQ ID NO: 59 - DNA, 21 bp, Artificial
  Primer
tggtttagcagagagcacatg

SEQ ID NO: 60 - DNA, 18 bp, Artificial
  Primer
gctcctagccctgcacac

SEQ ID NO: 61 - DNA, 24 bp, Artificial
  Primer
tggatttttaaacacagaatgtgg

SEQ ID NO: 62 - DNA, 25 bp, Artificial
  Primer
tcagcttctctttaaatttctctgg

SEQ ID NO: 63 - DNA, 22 bp, Artificial
  Primer
agccagtttcttcaaatcaacc

SEQ ID NO: 64 - DNA, 20 bp, Artificial
  Primer
atggttccgcagagaaacag

SEQ ID NO: 65 - DNA, 23 bp, Artificial
  Primer
ccaagggaggaaaaataagttaa

SEQ ID NO: 66 - DNA, 22 bp, Artificial
  Primer
accagcagtaggttgaggttaa

SEQ ID NO: 67 - DNA, 20 bp, Artificial
  Primer
aaccttcacaggctccttcc

SEQ ID NO: 68 - DNA, 20 bp, Artificial
  Primer
cccatctcttgtgccaaatc

SEQ ID NO: 69 - DNA, 20 bp, Artificial
  Primer
catctgaatggccagaatga

SEQ ID NO: 70 - DNA, 20 bp, Artificial
  Primer
gtcccctgcatgtgtctctc

SEQ ID NO: 71 - DNA, 21 bp, Artificial
  Primer
acattggcttacgctcacact

SEQ ID NO: 72 - DNA, 20 bp, Artificial
  Primer
cctgtctgggtttgtttgct

SEQ ID NO: 73 - DNA, 20 bp, Artificial
  Primer
atggacagaacagcctgaca

SEQ ID NO: 74 - DNA, 20 bp, Artificial
  Primer
tggtgaactcagcctcactg

SEQ ID NO: 75 - DNA, 20 bp, Artificial
  Primer
ttacttttcgtgggccagag

SEQ ID NO: 76 - DNA, 20 bp, Artificial
  Primer
ggaactgtgccacatagcaa

SEQ ID NO: 77 - DNA, 20 bp, Artificial
  Primer
tcttggaaaggggaaaaagc

SEQ ID NO: 78 - DNA, 24 bp, Artificial
  Primer
tgcttcatagcacttatctcttca

SEQ ID NO: 79 - DNA, 25 bp, Artificial
  Primer
agtaaggcctgcagtatttatatcc

SEQ ID NO: 80 - DNA, 25 bp, Artificial
  Primer
aatctttccctagaacttacaaagg

SEQ ID NO: 81 - DNA, 24 bp, Artificial
  Primer
ctgaaacactctaaaagggtatgc

SEQ ID NO: 82 - DNA, 21 bp, Artificial
  Primer
atcccaacatccacccattcc

SEQ ID NO: 83 - DNA, 21 bp, Artificial
  Primer
gttctgaggtttgtaaagccc

SEQ ID NO: 84 - DNA, 21 bp, Artificial
  Primer
ggtgagctacaatccataggg

SEQ ID NO: 85 - DNA, 23 bp, Artificial
  Primer
ccagcagaagagaaagatactga

SEQ ID NO: 86 - DNA, 23 bp, Artificial
  Primer
agtggtagaacttccatctcaca

SEQ ID NO: 87 - DNA, 27 bp, Artificial
  Primer
aatccagtgtgtgaaagactaatccag

SEQ ID NO: 88 - DNA, 32 bp, Artificial
  Primer
gtagatcaagatatagaatatttttcaacacc

SEQ ID NO: 89 - DNA, 20 bp, Artificial
  Primer
tgtctgtatttctgctgtgg

SEQ ID NO: 90 - DNA, 20 bp, Artificial
  Primer
acacggaagcgatctaaacg

SEQ ID NO: 91 - DNA, 18 bp, Artificial
  Primer
agtgccaaaaggaagcgc

SEQ ID NO: 92 - DNA, 19 bp, Artificial
  Primer
gacttgaccgttccacctg

SEQ ID NO: 93 - DNA, 19 bp, Artificial
  Primer
ccaaggtcattgttgcagc

SEQ ID NO: 94 - DNA, 20 bp, Artificial
  Primer
tggggatttgcttctcagtc

SEQ ID NO: 95 - DNA, 22 bp, Artificial
  Primer
taataaaactggtccctctggc

SEQ ID NO: 96 - DNA, 19 bp, Artificial
  Primer
tgctggctctccagtatgc

SEQ ID NO: 97 - DNA, 20 bp, Artificial
  Primer
tctcctggctacagggctaa

SEQ ID NO: 98 - DNA, 18 bp, Artificial
  Primer
cccactggcctagaaccc

SEQ ID NO: 99 - DNA, 20 bp, Artificial
  Primer
ggtgtgttggttaagactgg

SEQ ID NO: 100 - DNA, 20 bp, Artificial
  Primer
gaatcatagacctgacttcc

SEQ ID NO: 101 - DNA, 20 bp, Artificial
  Primer
tggacaggactgagtatgca

SEQ ID NO: 102 - DNA, 22 bp, Artificial
  Primer
aggtatccaactaacacagcca

SEQ ID NO: 103 - DNA, 21 bp, Artificial
  Primer
agcacctgtaccatctgttcc

SEQ ID NO: 104 - DNA, 22 bp, Artificial
  Primer
tccataggctcacaaagagttg

SEQ ID NO: 105 - DNA, 22 bp, Artificial
  Primer
tccctctaccatatatttcccc

SEQ ID NO: 106 - DNA, 22 bp, Artificial
  Primer
cattagcatggaaaaacctctg

SEQ ID NO: 107 - DNA, 20 bp, Artificial
  Primer
ccactacttgcctcagggag

SEQ ID NO: 108 - DNA, 21 bp, Artificial
  Primer
acaggactctcaagcatcagc

SEQ ID NO: 109 - DNA, 23 bp, Artificial
  Primer
gatgatccaccataactaccaga

SEQ ID NO: 110 - DNA, 20 bp, Artificial
  Primer
tggctctcaaaggtcattgt

SEQ ID NO: 111 - DNA, 24 bp, Artificial
  Primer
tacattaaccccaaaattaaatgc

SEQ ID NO: 112 - DNA, 24 bp, Artificial
  Primer
cccttgatttctctcatgagtatt

SEQ ID NO: 113 - DNA, 21 bp, Artificial
  Primer
tgtgtgctctctcacacatgc

SEQ ID NO: 114 - DNA, 19 bp, Artificial
  Primer
aaccaaagcagggatcagg

SEQ ID NO: 115 - DNA, 25 bp, Artificial
  Primer
tgcaaacatccacgtagcataaata

SEQ ID NO: 116 - DNA, 24 bp, Artificial
  Primer
gcatgaacagccaatagtgtcatc

SEQ ID NO: 117 - DNA, 20 bp, Artificial
  Primer
gaaagatgttgctagtgggg

SEQ ID NO: 118 - DNA, 20 bp, Artificial
  Primer
attctcctgtgaacctctcc

SEQ ID NO: 119 - DNA, 20 bp, Artificial
  Primer
ctagctgctggctacttggg

SEQ ID NO: 120 - DNA, 18 bp, Artificial
  Primer
ggctgctctgggctattg

SEQ ID NO: 121 - DNA, 19 bp, Artificial
  Primer
gtggtggcaaagagtcaga

SEQ ID NO: 122 - DNA, 19 bp, Artificial
  Primer
aacagccctgatttccata

SEQ ID NO: 123 - DNA, 21 bp, Artificial
  Primer
ttgtccagcccagcatttagc

SEQ ID NO: 124 - DNA, 19 bp, Artificial
  Primer
ggagaagacttatgggagc

SEQ ID NO: 125 - DNA, 23 bp, Artificial
  Primer
tgatattcaaactcaatgaaccc

SEQ ID NO: 126 - DNA, 23 bp, Artificial
  Primer
cttgcatatgctcatcattatca

SEQ ID NO: 127 - DNA, 18 bp, Artificial
  Primer
gaccactggaccaccagg

SEQ ID NO: 128 - DNA, 21 bp, Artificial
  Primer
ctggtaaaaagcaatgatgcc

SEQ ID NO: 129 - DNA, 23 bp, Artificial
  Primer
gagccaattcctacaattctctt

SEQ ID NO: 130 - DNA, 21 bp, Artificial
  Primer
agacatggctgaaatgactga

SEQ ID NO: 131 - DNA, 19 bp, Artificial
  Primer
cctctgtgtccacactgcc

SEQ ID NO: 132 - DNA, 20 bp, Artificial
  Primer
cctagtgacatcctggggtg

SEQ ID NO: 133 - DNA, 22 bp, Artificial
  Primer
acgcagaccttagcataggaga

SEQ ID NO: 134 - DNA, 22 bp, Artificial
  Primer
gtcgcaatgagttggacacaac

SEQ ID NO: 135 - DNA, 22 bp, Artificial
  Primer
ccagagaagcagaaccagtagg

SEQ ID NO: 136 - DNA, 21 bp, Artificial
  Primer
cttgtcagcgtccataattcc

SEQ ID NO: 137 - DNA, 24 bp, Artificial
  Primer
aaaactctgggaacttgaaaacta

SEQ ID NO: 138 - DNA, 24 bp, Artificial
  Primer
gttacaaatttaagagacagagtt

SEQ ID NO: 139 - DNA, 21 bp, Artificial
  Primer
ccaggtgggaataatgtctcc

SEQ ID NO: 140 - DNA, 20 bp, Artificial
  Primer
ttgggagcctgtggtttatc

SEQ ID NO: 141 - DNA, 18 bp, Artificial
  Primer
gctgtggctgtgcttgtg

SEQ ID NO: 142 - DNA, 18 bp, Artificial
  Primer
agtaactgcccccggaag

SEQ ID NO: 143 - DNA, 19 bp, Artificial
  Primer
tccctgtgttgaggagctg

SEQ ID NO: 144 - DNA, 20 bp, Artificial
  Primer
ttaatcaatgcacacgcatg

SEQ ID NO: 145 - DNA, 20 bp, Artificial
  Primer
tccttgtattcacacgtggg

SEQ ID NO: 146 - DNA, 20 bp, Artificial
  Primer
ttctcagccaaagtcaaggg

SEQ ID NO: 147 - DNA, 20 bp, Artificial
  Primer
gggtgtgacattttgttccc

SEQ ID NO: 148 - DNA, 20 bp, Artificial
  Primer
ctgctcgccactagtccttc

SEQ ID NO: 149 - DNA, 25 bp, Artificial
  Primer
gaatcaataaagaagatgcagcacg

SEQ ID NO: 150 - DNA, 25 bp, Artificial
  Primer
gccctctagctctatctgtgtttgc

SEQ ID NO: 151 - DNA, 22 bp, Artificial
  Primer
ttaaggttgttgcatactcctg

SEQ ID NO: 152 - DNA, 22 bp, Artificial
  Primer
aagttctcagccaaagtcaagg

SEQ ID NO: 153 - DNA, 24 bp, Artificial
  Primer
cccactttctggaaggcagaaatg

SEQ ID NO: 154 - DNA, 24 bp, Artificial
  Primer
cttattgtgttttctgccagggag

SEQ ID NO: 155 - DNA, 20 bp, Artificial
  Primer
gctacagcccttctggtttg

SEQ ID NO: 156 - DNA, 21 bp, Artificial
  Primer
gagctaatcaccaacagcaag

SEQ ID NO: 157 - DNA, 21 bp, Artificial
  Primer
tccacttcttccctcatctcc

SEQ ID NO: 158 - DNA, 21 bp, Artificial
  Primer
gaagtatatgtgtgcctggcc

SEQ ID NO: 159 - DNA, 20 bp, Artificial
  Primer
ggttcccagcagcagtagag

SEQ ID NO: 160 - DNA, 20 bp, Artificial
  Primer
cccatgtctcccgttacttc

SEQ ID NO: 161 - DNA, 21 bp, Artificial
  Primer
ggcaagctagagtcagacacg

SEQ ID NO: 162 - DNA, 22 bp, Artificial
  Primer
gcaaaagtctaggtgaaatgcc

SEQ ID NO: 163 - DNA, 23 bp, Artificial
  Primer
ccattaagaggaaattgtgttca

SEQ ID NO: 164 - DNA, 23 bp, Artificial
  Primer
atggagtcactgaaaggtactga

SEQ ID NO: 165 - DNA, 22 bp, Artificial
  Primer
gatcaccttgccactatttcct

SEQ ID NO: 166 - DNA, 21 bp, Artificial
  Primer
acatgacagccagctgctact

SEQ ID NO: 167 - DNA, 21 bp, Artificial
  Primer
taggctatgtactgaccatgc

SEQ ID NO: 168 - DNA, 21 bp, Artificial
  Primer
ctgaactgagatgactttggc

SEQ ID NO: 169 - DNA, 19 bp, Artificial
  Primer
cagctttccatcccctttc

SEQ ID NO: 170 - DNA, 20 bp, Artificial
  Primer
ctcccatcccaaacacagac

SEQ ID NO: 171 - DNA, 21 bp, Artificial
  Primer
ttctgaatttgattcccaaca

SEQ ID NO: 172 - DNA, 22 bp, Artificial
  Primer
actgtttccttaaaagcttccc

SEQ ID NO: 173 - DNA, 25 bp, Artificial
  Primer
ccttctctgaatttttgttgaaagc

SEQ ID NO: 174 - DNA, 22 bp, Artificial
  Primer
ggacagaagtgagtgactgaga

SEQ ID NO: 175 - DNA, 24 bp, Artificial
  Primer
ttggcacttactacctcatatgtt

SEQ ID NO: 176 - DNA, 22 bp, Artificial
  Primer
ttttctggatgttgagcctatt

SEQ ID NO: 177 - DNA, 20 bp, Artificial
  Primer
aaagactgcttgcctgaagc

SEQ ID NO: 178 - DNA, 22 bp, Artificial
  Primer
caaccagtgatgctgtactctg

SEQ ID NO: 179 - DNA, 22 bp, Artificial
  Primer
tcaaaaagttggacatgactga

SEQ ID NO: 180 - DNA, 24 bp, Artificial
  Primer
aggttttcaaatgagagacttttc

SEQ ID NO: 181 - DNA, 21 bp, Artificial
  Primer
gctcacaggttctgaggactc

SEQ ID NO: 182 - DNA, 22 bp, Artificial
  Primer
aacttgaagaaggaatgctgag

SEQ ID NO: 183 - DNA, 20 bp, Artificial
  Primer
actatggacatttggggcag

SEQ ID NO: 184 - DNA, 22 bp, Artificial
  Primer
agtaggtggagatcaaggatgc

SEQ ID NO: 185 - DNA, 22 bp, Artificial
  Primer
cagaccagtttctcagacaagc

SEQ ID NO: 186 - DNA, 22 bp, Artificial
  Primer
tcattcctgtgtcaatacagcc

SEQ ID NO: 187 - DNA, 25 bp, Artificial
  Primer
tgtatggctgaatgatattccattt

SEQ ID NO: 188 - DNA, 25 bp, Artificial
  Primer
ctactgacagatgattagataaaga

SEQ ID NO: 189 - DNA, 20 bp, Artificial
  Primer
taaggacttgagataaggag

SEQ ID NO: 190 - DNA, 20 bp, Artificial
  Primer
ccatctacctccatcttaac

SEQ ID NO: 191 - DNA, 22 bp, Artificial
  Primer
gacaaaacccttttagcacagg

SEQ ID NO: 192 - DNA, 22 bp, Artificial
  Primer
aattgcatggaaagttctcagc

SEQ ID NO: 193 - DNA, 23 bp, Artificial
  Primer
ttgtacatttctgtcaatgcctt

SEQ ID NO: 194 - DNA, 23 bp, Artificial
  Primer
acaatgtcattggtcaattcatt

SEQ ID NO: 195 - DNA, 21 bp, Artificial
  Primer
aacccaaatatccatcaatag

SEQ ID NO: 196 - DNA, 21 bp, Artificial
  Primer
cagtccctcaaccctcttttc

SEQ ID NO: 197 - DNA, 20 bp, Artificial
  Primer
tagtgtccaccagagacccc

SEQ ID NO: 198 - DNA, 22 bp, Artificial
  Primer
ccaaagacacagtttaaagggc

SEQ ID NO: 199 - DNA, 20 bp, Artificial
  Primer
ccagcatcaactgtcagagc

SEQ ID NO: 200 - DNA, 20 bp, Artificial
  Primer
ggcagattctttgccttctg

SEQ ID NO: 201 - DNA, 18 bp, Artificial
  Primer
aggtgctggaatggcaac

SEQ ID NO: 202 - DNA, 21 bp, Artificial
  Primer
tgtgattttggtcttccttgc

SEQ ID NO: 203 - DNA, 21 bp, Artificial
  Primer
tttctgctaatggcatcattt

SEQ ID NO: 204 - DNA, 22 bp, Artificial
  Primer
tggataaagaagatgtggtgtg

SEQ ID NO: 205 - DNA, 20 bp, Artificial
  Primer
aggaacccataggcagacac

SEQ ID NO: 206 - DNA, 20 bp, Artificial
  Primer
gcctggcaaagagaaaattc

(19) United States
(12) Patent Application Publication
(10) Pub. No.: US 2009/0176224 A1
(43) Pub. Date: Jul. 9, 2009

(54) Udder Health Characteristics

(75) Inventors: Mogens Sando Lund, Tjele (DK); Christian Bendixen, Ulstrup (DK); Helle Jensen, Viborg (DK); Bo Thomsen, &Aring;rhus (DK); Peter Sorensen, Viborg (DK); Soren Svendsen, Randers (DK); Bart Albert Johannes Buitenhuis, Tjele (DK); Vivi Hunnicke Nielsen, Tjele (DK); Bente Flugel Majgren, Hobro (DK); Bernt Guldbrandsten, Arhus (DK); Jorn Rind Thomasen, Holstebro (DK)

Correspondence Address: WEINGARTEN, SCHURGIN, GAGNEBIN & LEBOVICI LLP, TEN POST OFFICE SQUARE, BOSTON, MA, 02109, US

(73) Assignee: KVAEGAVLSFORENINGEN DANSIRE, Randers (DK)

(21) Appl. No.: 12/223,678

(22) PCT Filed: Feb. 5, 2007
(86) PCT No.: PCT/DK2007/000055

§ 371 Date: Nov. 6, 2008

(30) Foreign Application Priority Data
Feb. 6, 2006 (DK) PA 2006 00161
Dec. 22, 2006 (DK) PA 2006 01703

(51) Int. Cl. C12Q 1/68

(52) U.S. Cl. 435/6

(57) ABSTRACT

The invention relates to a method for determining udder health characteristics in bovine subjects, wherein udder health characteristics comprise sub-clinical and clinical mastitis. In particular, the method of the invention involves identification of genetic markers and/or Quantitative Trait Locus (QTL) for the determination of udder health characteristics in a bovine subject. The determination of udder health characteristics involves resolution of the specific microsatellite status. Furthermore, the invention relates to a diagnostic kit for detection of genetic marker(s) associated with udder health. The method and kit of the present invention can be applied for selection of bovine subjects for breeding purposes. Thus, the invention provides a method of genetically selecting bovine subjects with udder health characteristics that will yield cows less prone to mastitis.

### FIELD OF INVENTION

The present invention relates to udder health characteristics in bovine subjects. In particular, the invention relates to genetic markers for the determination of udder health characteristics in a bovine subject and a diagnostic kit for detection of genetic marker(s) associated with udder health.

### BACKGROUND OF INVENTION

Mastitis is the inflammation of the mammary gland or udder of the cow resulting from infection or trauma and is believed to be the most economically important disease in cattle.

The disease may be caused by a variety of agents. The primary cause of mastitis is the invasion of the mammary gland via the teat end by microorganisms.

The shape and structure of the teat are known to be influenced by hereditary factors (Hickman, 1964). A significant difference between dairy cattle with regard to the presence of mastitis was revealed by mastitis histories of two cow families in different geographical locations. Upon the findings it was concluded that heredity played a part in the infection rate. Also dam-daughter comparisons based on data derived from field surveys cite the influence of heredity on mastitis (Randel and Sunberg, 1962).

Mastitis may be clinical or sub-clinical, with sub-clinical infection preceding clinical manifestations. Clinical mastitis can be detected visually through observing red and swollen mammary glands i.e. red swollen udder, and through the production of clotted milk. Once detected, the milk from mastitic cows is kept separate from the vat so that it will not affect the overall milk quality.

Sub-clinical mastitis cannot be detected visually by swelling of the udder or by observation of the gland or the milk produced. Because of this, farmers do not have the option of diverting milk from sub-clinical mastitic cows. However, this milk is of poorer quality than that from non-infected cows and can thus contaminate the rest of the milk in the vat.

Sub-clinical and clinical mastitis can be detected by the use of somatic cell counts in which a sample of milk from a cow is analysed for the presence of somatic cells (white blood cells). Somatic cells are part of the cow's natural defense mechanism and cell counts rise when the udder becomes infected. The number of somatic cells in a milk sample can be estimated indirectly by rolling-ball viscometer and Coulter counter.

As mastitis results in reduced quantity and quality of milk and products from milk, mastitis results in economic losses to the farmer and dairy industry. Therefore, the ability to determine the genetic basis of bovine udder health is of immense economic significance to the dairy industry both in terms of daily milk production but also in breeding management, selecting for bovine subjects with preferred udder health characteristics. A method of genetically selecting bovine subjects with udder health characteristics that will yield cows less prone to mastitis would be desirable.

One approach to identify genetic determinants for genetic traits is the use of linkage disequilibrium (LD) mapping which aims at exploiting historical recombinants and has been shown in some livestock populations, including dairy cattle, to extend over very long chromosome segments when compared to human populations (Farnir et al., 2000). However, long range LD is likely to result in a limited mapping resolution and the occurrence of association in the absence of linkage due to gametic association between non syntenic loci. Once mapped, a Quantitative Trait Locus (QTL) can be usefully applied in marker assisted selection.

### Linkage Disequilibrium

Linkage disequilibrium reflects recombination events dating back in history and the use of LD mapping within families increases the resolution of mapping. LD exists when observed haplotypes in a population do not agree with the haplotype frequencies predicted by multiplying together the frequency of individual genetic markers in each haplotype. In this respect the term haplotype means a set of closely linked genetic markers present on one chromosome which tend to be inherited together. In order for LD mapping to be efficient the density of genetic markers needs to be compatible with the distance across which LD extends in the given population. In a study of LD in dairy cattle population using a high number of genetic markers (284 autosomal microsatellite markers) it was demonstrated that LD extends over several tens of centimorgans for intrachromosomal markers (Farnir et al. 2000). Similarly, Georges, M (2000) reported that the location of a genetic marker that is linked to a particular phenotype in livestock typically has a confidence interval of 20-30 cM (corresponding to maybe 500-1000 genes) (Georges, M., 2000). The existence of linkage disequilibrium is taken into account in order to use maps of particular regions of interest with high confidence.

The present invention offers a method of determining the genetic determinants for udder health traits of a given bovine subject which is of significant economic interest within the cattle industry.

In the present invention quantitative trait loci with pleiotropic effects on udder health traits have been mapped to chromosomes BTA1, BTA5, BTA6, BTA7, BTA9, BTA11, BTA15, BTA21, BTA26 and BTA27.

### SUMMARY OF INVENTION

It is an object of the present invention to provide an application method for marker assisted selection of polymorphisms in the bovine genome which polymorphisms are associated with udder health characteristics; and/or to provide genetic markers for use in such a method; and/or to provide animals selected using the method of the invention.

The identification of genetic markers that are linked to a particular phenotype, such as udder health, or to a heritable disease has been facilitated by the discovery of microsatellite markers as a source of polymorphic markers and single nucleotide polymorphisms linked to a mutation causing a specific phenotype. Markers linked to the mutation or the mutation itself causing a specific phenotype of interest are localised by use of genetic analysis in pedigrees and also by exploiting linkage disequilibrium when looking at populations.

One aspect of the present invention thus relates to a method for determining udder health characteristics in a bovine subject, comprising detecting in a sample from said bovine subject the presence or absence of at least one genetic marker that is linked to at least one trait indicative of udder health, wherein said at least one genetic marker is located on the bovine chromosome BTA1 in the region flanked by and including the polymorphic microsatellite markers BMS4008 and URB014 and/or BTA5 in the region flanked by and including the polymorphic microsatellite markers BMS1095 and BM315 and/or BTA6 in the region flanked by and including the polymorphic microsatellite markers ILSTS093 and BL1038 and/or BTA7 in the region flanked by and including the polymorphic microsatellite markers BM7160 and BL1043 and/or BTA9 in the region flanked by and including the polymorphic microsatellite markers BMS2151 and BMS1967 and/or BTA11 in the region flanked by and including the polymorphic microsatellite markers BM716 and HEL13 and/or BTA15 in the region flanked by and including the polymorphic microsatellite markers BMS2684 and BMS429 and/or BTA21 in the region flanked by and including the polymorphic microsatellite markers BMS1117 and BM846 and/or BTA26 in the region flanked by and including the polymorphic microsatellite markers BMS651 and BM7237 and/or BTA27 in the region flanked by and including the polymorphic microsatellite markers BMS1001 and BM203, wherein the presence or absence of said at least one genetic marker is indicative of udder health characteristics of said bovine subject or off-spring therefrom.

Another aspect of the present invention relates to a diagnostic kit for use in detecting the presence or absence in a bovine subject of at least one genetic marker associated with bovine udder health, comprising at least one oligonucleotide sequence and combinations thereof, wherein the nucleotide sequences are selected from any of SEQ ID NO.: 1 to SEQ ID NO.:206 and/or any combination thereof.

### DESCRIPTION OF DRAWINGS

FIG. 1: Genome scan of BTA1 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 2: Genome scan of BTA1 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 3: Genome scan of BTA5 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 4: Genome scan of BTA5 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 5: Genome scan of BTA7 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 6: Genome scan of BTA7 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 7: Genome scan of BTA15 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 8: Genome scan of BTA15 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 9: Genome scan of BTA21 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 10: Genome scan of BTA21 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 11: Genome scan of BTA27 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 12: Genome scan of BTA27 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 13: Genome scan of BTA6 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 14: Genome scan of BTA9 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 15: Genome scan of BTA9 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 16: Genome scan of BTA11 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 17: Genome scan of BTA26 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 18: Genome scan of BTA26 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

FIG. 19: Genome scan of BTA26 in relation to udder health characteristics. Numbers refer to ‘herdbook number’ and udder health parameter, respectively. The X-axis represents the distance of the chromosome expressed in Morgan according to the positions employed in this analysis. The Y-axis represents the test-statistics of the QTL analysis expressed in the F-value.

### DETAILED DESCRIPTION OF THE INVENTION

The present invention relates to genetic determinants of udder health in dairy cattle. The occurrence of mastitis, both clinical and sub-clinical mastitis involves substantial economic loss for the dairy industry. Therefore, it is of economic interest to identity those bovine subjects that have a genetic predisposition for developing mastitis. Bovine subjects with such genetic predisposition are carriers of non-desired traits, which can be passed on to their offspring.

The term “bovine subject” refers to cattle of any breed and is meant to include both cows and bulls, whether adult or newborn animals. No particular age of the animals are denoted by this term. One example of a bovine subject is a member of the Holstein breed. In one preferred embodiment, the bovine subject is a member of the Holstein-Friesian cattle population. In another embodiment, the bovine subject is a member of the Holstein Swartbont cattle population. In another embodiment, the bovine subject is a member of the Deutsche Holstein Schwarzbunt cattle population. In another embodiment, the bovine subject is a member of the US Holstein cattle population. In one embodiment, the bovine subject is a member of the Red and White Holstein breed. In another embodiment, the bovine subject is a member of the Deutsche Holstein Schwarzbunt cattle population. In one embodiment, the bovine subject is a member of any family, which include members of the Holstein breed. In one embodiment the bovine subject is a member of the Danish Red population. In another embodiment the bovine subject is a member of the Finnish Ayrshire population. In yet another embodiment the bovine subject is a member of the Swedish Red population. In a further embodiment the bovine subject is a member of the Danish Holstein population. In another embodiment, the bovine subject is a member of the Swedish Red and White population. In yet another embodiment, the bovine subject is a member of the Nordic Red population.

In one embodiment of the present invention, the bovine subject is selected from the group consisting of Swedish Red and White, Danish Red, Finnish Ayrshire, Holstein-Friesian, Danish Holstein and Nordic Red. In another embodiment of the present invention, the bovine subject is selected from the group consisting of Finnish Ayrshire and Swedish Red cattle. In another embodiment of the present invention, the bovine subject is selected from the group consisting of Finnish Ayrshire and Swedish Red cattle.

In one embodiment, the bovine subject is selected from the group of breeds shown in table 1a1

TABLE 1a1
Breed names and breed codes assigned by ICAR
(International Committee for Animal Recording)

| | Breed | National Breed |
|---|---|---|
| Breed | Code | Names Annex |
| Abondance | AB | — |
| Tyrol Grey | AL | 2.2 |
| Angus | AN | 2.1 |
| Aubrac | AU | |
| Ayrshire | AY | 2.1 |
| Belgian Blue | BB | |
| Blonde d'Aquitaine | BD | |
| Beefmaster | BM | |
| Braford | BO | |
| Bralunan | BR | |
| Brangus | BN | |
| Brown Swiss | BS | 2.1 |
| Chianina | CA | |
| Charolais | CH | |
| Dexter | DR | |
| Galloway | GA | 2.2 |
| Guernsey | GU | |
| Gelbvieh | GV | |
| Hereford, horned | HH | |
| Hereford, polled | HP | |
| Highland Cattle | HI | |
| Holstein | HO | 2.2 |
| Jersey | JE | |
| Limousin | LM | |
| Maine-Anjou | MA | |
| Murray-Grey | MG | |
| Montbéliard | MO | |
| Marchigiana | MR | |
| Normandy | NO** | |
| Piedmont | PI | 2.2 |
| Pinzgau | PZ | |
| European Red Dairy Breed | [RE]* | 2.1, 2.2 |
| Romagnola | RN | |
| Holstein, Red and White | RW*** | 2.2 |
| Salers | SL** | |
| Santa Gertrudis | SG | |
| South Devon | SD | |
| Shorthorn | [SH]* | 2.2 |
| Simmental | SM | 2.2 |
| Sahiwal | SW | |
| Tarentaise | TA | |
| Welsh Black | WB | |
| Buffalo (Bubalis bubalis) | BF | |

*new breed code
**change from earlier code because of existing code in France
***US proposal WW

In one embodiment, the bovine subject is a member of a breed selected from the group of breeds shown in table 1a2

TABLE 1a2
Breed names

| National Breed Names | | |
|---|---|---|
| English Name | National names | |
| Angus | Including | Aberdeen Angus |
| | | Canadian Angus |
| | | American Angus |
| | | German Angus |
| Ayrshire | Including | Ayrshire in |
| | | Australia |
| | | Canada |
| | | Colombia |
| | | Czech Republic |
| | | Finland |
| | | Kenya |
| | | New Zealand |
| | | Norway (NRF) |
| | | Russia |
| | | South Africa |
| | | Sweden (SRB) and SAB |
| | | UK |
| | | US |
| | | Zimbabwe |
| Belgian Blue | French: | Blanc-bleu Belge |
| | Flemish: | Witblauw Ras van Belgie |
| Brown Swiss | German: | Braunvieh |
| | Italian: | Razza Bruna |
| | French: | Brune |
| | Spanish: | Bruna, Parda Alpina |
| | Serbo-Croatian: | Slovenacko, belo |
| | Czech: | Hnady Karpatsky |
| | Romanian: | Shivitskaja |
| | Russian: | Bruna |
| | Bulgarian: | Bljarska kafyava |
| European Red Dairy Breed | Including | Danish Red |
| | | Angeln |
| | | Swedish Red and White |
| | | Norwegian Red and White |
| | | Estonian Red |
| | | Latvian Brown |
| | | Lithuanian Red |
| | | Byelorus Red |
| | | Polish Red Lowland |

In one embodiment, the bovine subject is a member of a breed selected from the group of breeds shown in table 1a3

TABLE 1a3
Breed names
National Breed Names

| English Name | National names | |
|---|---|---|
| European Red Dairy Breed | | Ukrainian Polish Red |
| (continued) | | (French Rouge Flamande?) |
| | | (Belgian Flamande Rouge?) |
| Galloway: | Including | Black and Dun |
| | | Galloway |
| | | Belted Galloway |
| | | Red Galloway |
| | | White Galloway |
| Holstein, Black and White: | Dutch: | Holstein Swartbont |
| | German: | Deutsche Holstein, schwarzbunt |
| | Danish: | Sortbroget Dansk Malkekvaeg |
| | British: | Holstein Friesian |
| | Swedish: | Svensk Låglands Boskaap |
| | French: | Prim Holstein |
| | Italian: | Holstein Frisona |
| | Spanish: | Holstein Frisona |
| Holstein, Red and White | Dutch: | Holstein, roodbunt |
| | German: | Holstein, rotbunt |
| | Danish: | Roedbroget Dansk Malkekvaeg |
| Piedmont | Italian: | Piemontese |
| Shorthorn | Including | Dairy Shorthorn |
| | | Beef Shorthorn |
| | | Polled Shorthorn |
| Simmental | Including dual purpose and beef use | |
| | German: | Fleckvieh |
| | French: | Simmental Française |
| | Italian: | Razza Pezzata Rossa |
| | Czech: | Cesky strakatý |
| | Slovakian: | Slovensky strakaty |
| | Romanian: | Baltata româneasca |
| | Russian: | Simmentalskaja |
| Tyrol Grey | German: | Tiroler Grauvieh |
| | | Oberinntaler Grauvieh |
| | | Rätisches Grauvieh |
| | Italian: | Razza Grigia Alpina |

The term “genetic marker” refers to a variable nucleotide sequence (polymorphism) of the DNA on the bovine chromosome and distinguishes one allele from another. The variable nucleotide sequence can be identified by methods known to a person skilled in the art for example by using specific oligonucleotides in for example amplification methods and/or observation of a size difference. However, the variable nucleotide sequence may also be detected by sequencing or for example restriction fragment length polymorphism analysis. The variable nucleotide sequence may be represented by a deletion, an insertion, repeats, and/or a point mutation.

One type of genetic marker is a microsatellite marker that is linked to a quantitative trait locus. Microsatellite markers refer to short sequences repeated after each other. In short sequences are for example one nucleotide, such as two nucleotides, for example three nucleotides, such as four nucleotides, for example five nucleotides, such as six nucleotides, for example seven nucleotides, such as eight nucleotides, for example nine nucleotides, such as ten nucleotides. However, changes sometimes occur and the number of repeats may increase or decrease. The specific definition and locus of the polymorphic microsatellite markers can be found in the USDA genetic map (Kappes et al. 1997; or by following the link to U.S. Meat Animal Research Center http://www.marc.usda.gov/).

It is furthermore appreciated that the nucleotide sequences of the genetic markers of the present invention are genetically linked to traits for udder health in a bovine subject. Consequently, it is also understood that a number of genetic markers may be generated from the nucleotide sequence of the DNA region(s) flanked by and including the genetic markers according to the method of the present invention.

### Udder Health Characteristics

Udder health of a bovine subject is affected by a number of characteristics. Traits that affect the udder health according to the present invention are for example the occurrence of clinical mastitis, somatic cell counts (SCC) and udder conformation. Herein the term SCC is identical to the term CELL. Somatic cell score (SCS) was defined as the mean of log10 transformed somatic cell count values (in 10,000/mL) obtained from the milk recording scheme. The mean was taken over the period 10 to 180 after calving. By the term udder health characteristics is meant traits, which affect udder health in the bovine subject or its off-spring. Thus, udder health characteristics of a bull are physically manifested by its female off-spring.

In the present invention the traits Mas1, Mas2 (CM1), Mas3 (CM2), Mas4 (CM3), SCC and udder health are used which refer to the following characteristics:

Mas1: Treated cases of clinical mastitis in the period −5 to 50 days after 1^{st} calving.

Mas2 (also designated CM1): Treated cases of clinical mastitis in the period −5 to 305 days after 1^{st} calving.

Mas3 (also designated CM2): Treated cases of clinical mastitis in the period −5 to 305 days after 2^{nd} calving.

Mas4 (also designated CM3): Treated cases of clinical mastitis in the period −5 to 305 days after 3^{rd} or later calving.

SCS: Mean SCS in period 5-180 days after 1^{st} calving.

Udder health index: An index weighing together information from Mas1-Mas4, SCC, fore udder attachment, udder depth, and udder band.

In one embodiment of the present invention, the method and kit described herein relates to udder health index. In another embodiment of the present invention, the method and kit described herein relates to clinical mastitis. In another embodiment, the method and kit of the present invention pertains to sub-clinical mastitis, such as detected by somatic cell counts. In yet another embodiment, the method and kit of the present invention primarily relates to clinical mastitis in combination with sub-clinical mastitis such as detected by somatic cell counts.

Registrations from daughters of bulls were examined and used in establishing a relation between the observable incidents of mastitis and potential genetic determinants of udder health in a bovine subject, see Table 16.

### Granddaughter Design

The granddaughter design includes analysing data from DNA-based markers for grandsires that have been used extensively in breeding and for sons of grandsires where the sons have produced offspring. The phenotypic data that are to be used together with the DNA-marker data are derived from the daughters of the sons. Such phenotypic data could be for example milk production features, features relating to calving, meat quality, or disease. One group of daughters has inherited one allele from their father whereas a second group of daughters has inherited the other allele from their father. By comparing data from the two groups information can be gained whether a fragment of a particular chromosome is harbouring one or more genes that affect the trait in question. It may be concluded whether a QTL is present within this fragment of the chromosome.

A prerequisite for performing a granddaughter design is the availability of detailed phenotypic data. In the present invention such data have been available to the inventors (http://www.ir.dk/kvaeg/diverse/principles.pdf).

QTL is a short form of quantitative trait locus. Genes conferring quantitative traits to an individual may be found in an indirect manner by observing pieces of chromosomes that act as if one or more gene(s) is located within that piece of the chromosome.

In contrast, DNA markers can be used directly to provide information of the traits passed on from parents to one or more of their offspring when a number of DNA markers on a chromosome has been determined for one or both parents and their offspring. The markers may be used to calculate the genetic history of the chromosome linked to the DNA markers.

### Frequency of Recombination

The frequency of recombination is the likelihood that a recombination event will occur between two genes or two markers. The frequency of recombination may be calculated as the genetic distance between the two genes or the two markers. Genetic distance is measured in units of centiMorgan (cM). One centiMorgan is equal to a 1% chance that a marker at one genetic locus will be separated from a marker at a second locus due to crossing over in a single generation. One centiMorgan is equivalent, on average, to one million base pairs.

### Chromosomal Regions and Markers

BTA is short for Bos taurus autosome.

One aspect of the present invention relates to a method for determining udder health characteristics in a bovine subject, comprising detecting in a sample from said bovine subject the presence or absence of at least one genetic marker that is linked to at least one trait indicative of udder health, wherein said at least one genetic marker is located on the bovine chromosome BTA1 in the region flanked by and including the polymorphic microsatellite markers BMS4008 and URB014 and/or BTA5 in the region flanked by and including the polymorphic microsatellite markers BMS1095 and BM315 and/or BTA6 in the region flanked by and including the polymorphic microsatellite markers ILSTS093 and BL1038 and/or BTA7 in the region flanked by and including the polymorphic microsatellite markers BM7160 and BL1043 and/or BTA9 in the region flanked by and including the polymorphic microsatellite markers BMS2151 and BMS1967 and/or BTA11 in the region flanked by and including the polymorphic microsatellite markers BM716 and HEL13 and/or BTA15 in the region flanked by and including the polymorphic microsatellite markers BMS2684 and BMS429 and/or BTA21 in the region flanked by and including the polymorphic microsatellite markers BMS1117 and BM846 and/or BTA26 in the region flanked by and including the polymorphic microsatellite markers BMS651 and BM7237 and/or BTA27 in the region flanked by and including the polymorphic microsatellite markers BMS1001 and BM203, wherein the presence or absence of said at least one genetic marker is indicative of udder health characteristics of said bovine subject or off-spring therefrom.

In order to determine udder health characteristics in a bovine subject, wherein the at least one genetic marker is present located on a bovine chromosome in the region flanked by and including the polymorphic microsatellite marker, it is appreciated that more than one genetic marker may be employed in the present invention. For example the at least one genetic marker may be a combination of at least two or more genetic markers such that the accuracy may be increased, such as at least three genetic markers, for example four genetic markers, such as at least five genetic markers, for example six genetic markers, such as at least seven genetic markers, for example eight genetic markers, such as at least nine genetic markers, for example ten genetic markers.

The at least one genetic marker may be located on at least one bovine chromosome, such as two chromosomes, for example three chromosomes, such as four chromosomes, for example five chromosomes, and/or such as six chromosomes.

In a preferred embodiment the at least one marker is selected from any of the individual markers of the tables shown herein.

### BTA1

In one embodiment of the invention the at least one genetic marker is located on the bovine chromosome BTA1. In one specific embodiment of the present invention, the at least one genetic marker is located in the region from about 80,379 cM to about 154.672 cM (http://www.marc.usda.gov/) on the bovine chromosome BTA1. In one embodiment the at least one genetic marker is located on the bovine chromosome BTA1 in the region flanked by and including the markers BMS4008 and URB014. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index. However, in a further embodiment the at least one genetic marker is significant for the traits in any combination. The at least one genetic marker is selected from the group of markers shown in Table1b1:

TABLE 1b1

| Marker on | Position employed | Relative position (cM) |
|---|---|---|
| BTA1 | in analysis (cM) | http://www.marc.usda.gov/ |
| BMS4008 | 71.7 | 80.379 |
| BM8246 | 76.2 | 83.834 |
| BMS4031 | 77.7 | 87.124 |
| DIK2273 | 84.5 | 84.471 |
| DIK4151 | 90.0 | 89.989 |
| MCM130 | 92.6 | 92.649 |
| DIK4367 | 97.2 | 97.246 |
| TGLA130 | 98.2 | 110.816 |
| BMS1789 | 100.9 | 113.501 |
| CSSM019 | 108.3 | 122.094 |
| BM1824 | 108.6 | 122.391 |
| UWCA46 | 113.2 | 127.441 |
| BMS918 | 118.1 | 132.471 |
| BMS4043 | 128.7 | 142.244 |
| URB014 | 142.1 | 154.672 |

In a preferred embodiment of the invention, the at least one genetic marker is located in the region from about 89.989 cM to about 113.501 cM (http://www.marc.usda.gov/) on the bovine chromosome BTA1.

In one embodiment the at least one genetic marker is located on the bovine chromosome BTA1 in the region flanked by and including the markers DIK4151 and BMS1789. The at least one genetic marker is selected from the group of markers shown in Table 1b2:

TABLE 1b2

| Marker on | Position employed | Relative position (cM) |
|---|---|---|
| BTA1 | in analysis (cM) | http://www.marc.usda.gov/ |
| DIK4151 | 90.0 | 89.989 |
| MCM130 | 92.6 | 92.649 |
| DIK4367 | 97.2 | 97.246 |
| TGLA130 | 98.2 | 110.816 |
| BMS1789 | 100.9 | 113.501 |

In another preferred embodiment of the invention, the at least one genetic marker is located in the region from about 92.649 cM to about 110.816 cM (http://www.marc.usda.gov/) on the bovine chromosome BTA1.

In one embodiment the at least one genetic marker is located on the bovine chromosome BTA1 in the region flanked by and including the markers MCM130 and TGLA130. The at least one genetic marker is selected from the group of markers shown in Table 1b3:

TABLE 1b3

| Marker on | Position employed | Relative position (cM) |
|---|---|---|
| BTA1 | in analysis (cM) | http://www.marc.usda.gov/ |
| MCM130 | 92.6 | 92.649 |
| DIK4367 | 97.2 | 97.246 |
| TGLA130 | 98.2 | 110.816 |

In yet another preferred embodiment, the at least one genetic marker is located in the region from about 89.989 cM to about 97.246 cM on the bovine chromosome BTA1.

In one embodiment the at least one genetic marker is located on the bovine chromosome BTA1 in the region flanked by and including the markers DIK4151 and DIK4367. The at least one genetic marker is significant for the traits CELL, MAS1, MAS2.

The at least one genetic marker is selected from the group of markers shown in Table 1b4:

TABLE 1b4

| Marker on | Position employed | Relative position (cM) |
|---|---|---|
| BTA1 | in analysis (cM) | http://www.marc.usda.gov/ |
| DIK4151 | 90.0 | 89.989 |
| MCM130 | 92.6 | 92.649 |
| DIK4367 | 97.2 | 97.246 |

In an even more preferred embodiment, the at least one genetic marker is located in the region from about 92.649 cM to about 97.246 cM (http://www.marc.usda.gov/) on the bovine chromosome BTA1.

In one embodiment the at least one genetic marker is located on the bovine chromosome BTA1 in the region flanked by and including the markers MCM130 and DIK4367. The at least one genetic marker is selected from the group of markers shown in Table 1b5:

TABLE 1b5

| Marker on | Position employed | Relative position (cM) |
|---|---|---|
| BTA1 | in analysis (cM) | http://www.marc.usda.gov/ |
| MCM130 | 92.6 | 92.649 |
| DIK4367 | 97.2 | 97.246 |

In a further embodiment of the invention, the at least one genetic marker is located in the region from about 97.246 cM to about 132.471 cM (http://www.marc.usda.gov/) on the bovine chromosome BTA1. In one embodiment the at least one genetic marker is located on the bovine chromosome BTA1 in the region flanked by and including the markers DIK4367 and BMS918. The at least one genetic marker is selected from the group of markers shown in Table 1b6:

TABLE 1b6

| Marker on | Position employed | Relative position (cM) |
|---|---|---|
| BTA1 | in analysis (cM) | http://www.marc.usda.gov/ |
| DIK4367 | 97.2 | 97.246 |
| TGLA130 | 98.2 | 110.816 |
| BMS1789 | 100.9 | 113.501 |
| CSSM019 | 108.3 | 122.094 |
| BM1824 | 108.6 | 122.391 |
| UWCA46 | 113.2 | 127.441 |
| BMS918 | 118.1 | 132.471 |

In yet another embodiment of the invention, the at least one genetic marker is located in the region from about 132.471 cM to about 142.244 cM (http://www.marc.usda.gov/) on the bovine chromosome BTA1.

In one embodiment the at least one genetic marker is located on the bovine chromosome BTA1 in the region flanked by and including the markers BMS918 and BMS4043. The at least one genetic marker is selected from the group of markers shown in Table 1b7:

TABLE 1b7

| Marker on | Position employed | Relative position (cM) |
|---|---|---|
| BTA1 | in analysis (cM) | http://www.marc.usda.gov/ |
| BMS918 | 118.1 | 132.471 |
| BMS4043 | 128.7 | 142.244 |

In a further embodiment of the invention, the at least one genetic marker is located in the region from about 132.471 cM to about 154,672 cM (http://www.marc.usda.gov/) on the bovine chromosome BTA1.

In one embodiment the at least one genetic marker is located on the bovine chromosome BTA1 in the region flanked by and including the markers BMS918 and URBO14. The at least one genetic marker is selected from the group of markers shown in Table 1b8:

TABLE 1b8

| Marker on | Position employed | Relative position (cM) |
|---|---|---|
| BTA1 | in analysis (cM) | http://www.marc.usda.gov/ |
| BMS918 | 118.1 | 132.471 |
| BMS4043 | 128.7 | 142.244 |
| URBO14 | 142.1 | 154.672 |

### BTA5

In another embodiment of the invention the at least one genetic marker is located on the bovine chromosome BTA5. In one specific embodiment of the present invention, the at least one genetic marker is located in the region from about 0 cM to about 103.169 cM (http://www.marc.usda.gov/) on the bovine chromosome BTA5. In one embodiment the at least one genetic marker is located on the bovine chromosome BTA5 in the region flanked by and including the markers BMS1095 and BM315. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index.

However, in a further embodiment the at least one genetic marker is significant for the traits in any combination. The at least one genetic marker is selected from the group of markers shown in Table 2a:

TABLE 2a

| Marker on | Position employed | Relative position (cM) |
|---|---|---|
| BTA5 | in analysis (cM) | http://www.marc.usda.gov/ |
| BMS1095 | 0.0 | 0 |
| BM6026 | 6.7 | 6.05 |
| BMS610 | 12.8 | 12.018 |
| BP1 | 18.8 | 17.287 |
| DIK2718 | 30.1 | 30.143 |
| AGLA293 | 32.0 | 32.253 |
| DIK5002 | 33.7 | 33.655 |
| DIK4759 | 40.3 | 40.293 |
| BMC1009 | 40.6 | 41.693 |
| RM500 | 55.6 | 56.303 |
| ETH10 | 70.0 | 71.764 |
| CSSM022 | 72.4 | 74.2 |
| BMS1216 | 75.6 | 78.205 |
| BMS1248 | 88.4 | 90.849 |
| BM315 | 100.1 | 103.169 |

In one specific embodiment of the present invention, the at least one genetic marker is located in the region from about 33.655 cM to about 56.303 cM (http://www.marc.usda.gov/) on the bovine chromosome BTA5.

In one embodiment the at least one genetic marker is located on the bovine chromosome BTA5 in the region flanked by and including the markers DIK5002 and RM500. The at least one genetic marker is selected from the group of markers shown in Table 2b:

TABLE 2b

| Marker on | Position employed | Relative position (cM) |
|---|---|---|
| BTA5 | in analysis (cM) | http://www.marc.usda.gov/ |
| DIK5002 | 33.7 | 33.655 |
| DIK4759 | 40.3 | 40.293 |
| BMC1009 | 40.6 | 41.693 |
| RM500 | 55.6 | 56.303 |

In another specific embodiment, the at least one genetic marker is located in the region from about 40.293 cM to about 56.303 cM (http://www.marc.usda.gov/) on the bovine chromosome BTA5. In one embodiment the at least one genetic marker is located on the bovine chromosome BTA5 in the region flanked by and including the markers DIK4759 and RM500. The at least one genetic marker is selected from the group of markers shown in Table 2b1:

TABLE 2b1

| Marker on | Position employed | Relative position (cM) |
|---|---|---|
| BTA5 | in analysis (cM) | http://www.marc.usda.gov/ |
| DIK4759 | 40.3 | 40.293 |
| BMC1009 | 40.6 | 41.693 |
| RM500 | 55.6 | 56.303 |

In yet another specific embodiment of the present invention, the at least one genetic marker is located in the region from about 40.293 cM to about 41.693 cM (http://www.marc.usda.gov/) on the bovine chromosome BTA5.

In one embodiment the at least one genetic marker is located on the bovine chromosome BTA5 in the region flanked by and including the markers DIK4759 and BMC1009. The at least one genetic marker is selected from the group of markers shown in Table 2b2:

TABLE 2b2

| Marker on | Position employed | Relative position (cM) |
|---|---|---|
| BTA5 | in analysis (cM) | http://www.marc.usda.gov/ |
| DIK4759 | 40.3 | 40.293 |
| BMC1009 | 40.6 | 41.693 |

In a further embodiment of the present invention, the at least one genetic marker is located in the region from about 17.287 cM to about 40.293 cM (http://www.marc.usda.gov/) on the bovine chromosome BTA5.

In one embodiment the at least one genetic marker is located on the bovine chromosome BTA5 in the region flanked by and including the markers BPI and DIK4759. The at least one genetic marker is selected from the group of markers shown in Table 2c:

TABLE 2c

| Marker on | Position employed | Relative position (cM) |
|---|---|---|
| BTA5 | in analysis (cM) | http://www.marc.usda.gov/ |
| BP1 | 18.8 | 17.287 |
| DIK2718 | 30.1 | 30.143 |
| AGLA293 | 32.0 | 32.253 |
| DIK5002 | 33.7 | 33.655 |
| DIK4759 | 40.3 | 40.293 |

In yet a further embodiment of the present invention, the at least one genetic marker is located in the region from about 56.303 cM to about 71.764 cM (http://www.marc.usda.gov/) on the bovine chromosome BTA5.

In one embodiment the at least one genetic marker is located on the bovine chromosome BTA5 in the region flanked by and including the markers RM500 and ETH10. The at least one genetic marker is selected from the group of markers shown in Table 2d:

TABLE 2d

| Marker on | Position employed | Relative position (cM) |
|---|---|---|
| BTA5 | in analysis (cM) | http://www.marc.usda.gov/ |
| RM500 | 55.6 | 56.303 |
| ETH10 | 70.0 | 71.764 |

In a preferred embodiment the at least one genetic marker is RM500 positioned at bovine chromosome BTA5 at position 56.303 cM (http://www.marc.usda.gov/). In another preferred embodiment the at least one genetic marker is ETH10 located at bovine chromosome BTA5 at position 71.764. In yet another embodiment of the present invention, the at least one genetic marker is located in the region from about 41,693 cM to about 71.764 cM (http://www.marc.usda.gov/) on the bovine chromosome BTA5.

In one embodiment the at least one genetic marker is located on the bovine chromosome BTA5 in the region flanked by and including the markers BMC1009 and ETH10. The at least one genetic marker is selected from the group of markers shown in Table 2e:

TABLE 2e

| Marker | Position employed | Relative position (cM) |
|---|---|---|
| on BTA5 | in analysis (cM) | http://www.marc.usda.gov/ |
| BMC1009 | 40.6 | 41.693 |
| RM500 | 55.6 | 56.303 |
| ETH10 | 70.0 | 71.764 |

In a further embodiment of the present invention, the at least one genetic marker is located in the region from about 71.764 cM to about 78.205 (http://www.marc.usda.gov/) on the bovine chromosome BTA5.

In one embodiment the at least one genetic marker is located on the bovine chromosome BTA5 in the region flanked by and including the markers ETH10 and BMS1216. The at least one genetic marker is selected from the group of markers shown in Table 2f:

TABLE 2f

| Marker | Position employed | Relative position (cM) |
|---|---|---|
| on BTA5 | in analysis (cM) | http://www.marc.usda.gov/ |
| ETH10 | 70.0 | 71.764 |
| CSSM022 | 72.4 | 74.2 |
| BMS1216 | 75.6 | 78.205 |

### BTA6

In another embodiment of the invention the at least one genetic marker is located on the bovine chromosome BTA6. In one specific embodiment of the present invention, the at least one genetic marker is located in the region from about 0 cM to about 129.985 cM (http://www.marc.usda.gov/) on the bovine chromosome BTA6. In one embodiment the at least one genetic marker is located on the bovine chromosome BTA6 in the region flanked by and including the markers ILSTS093 and BL1038. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index. However, in a further embodiment the at least one genetic marker is significant for the traits in any combination. The at least one genetic marker is selected from the group of markers shown in Table 2g:

TABLE 2g

| Marker | Position employed | Relative position (cM) |
|---|---|---|
| on BTA6 | in analysis (cM) | http://www.marc.usda.gov/ |
| ILSTS093 | 0 | 0 |
| INRA133 | 8.2 | 8.053 |
| BM1329 | 35.5 | 35.398 |
| OARJMP36*¹ | 52.4 | 56.12 |
| BM415 | 76.3 | 81.961 |
| BM4311 | 89.1 | 97.728 |
| BM2320 | 120.7 | 127.264 |
| BL1038 | 122.3 | 129.985 |

*¹also known as JMP36

In one specific embodiment of the present invention, the at least one genetic marker is located in the region from about 56.12 cM to about 129.985 cM (http://www.marc.usda.gov/) on the bovine chromosome BTA6.

In one embodiment the at least one genetic marker is located on the bovine chromosome BTA6 in the region flanked by and including the markers OARJMP36 and BL1038. The at least one genetic marker is selected from the group of markers shown in Table 2g1:

TABLE 2g1

| | Position employed in | Relative position (cM) |
|---|---|---|
| Marker on BTA6 | analysis (cM) | http://www.marc.usda.gov/ |
| OARJMP36 | 52.4 | 56.12 |
| BM415 | 76.3 | 81.961 |
| BM4311 | 89.1 | 97.728 |
| BM2320 | 120.7 | 127.264 |
| BL1038 | 122.3 | 129.985 |

In one specific embodiment of the present invention, the at least one genetic marker is located in the region from about 56.12 cM to about 97.728 cM (http://www.marc.usda.gov/) on the bovine chromosome BTA6.

In one embodiment the at least one genetic marker is located on the bovine chromosome BTA6 in the region flanked by and including the markers OARJMP36 and BM4311. The at least one genetic marker is selected from the group of markers shown in Table 2g2:

TABLE 2g2

| | Position employed in | Relative position (cM) |
|---|---|---|
| Marker on BTA6 | analysis (cM) | http://www.marc.usda.gov/ |
| OARJMP36*¹ | 52.4 | 56.12 |
| BM415 | 76.3 | 81.961 |
| BM4311 | 89.1 | 97.728 |

In one specific embodiment of the present invention, the at least one genetic marker is located in the region from about 97.728 cM to about 127.264 cM (http://www.marc.usda.gov/) on the bovine chromosome BTA6.

In one embodiment the at least one genetic marker is located on the bovine chromosome BTA6 in the region flanked by and including the markers BM4311 and BM2320. The at least one genetic marker is selected from the group of markers shown in Table 2g3:

TABLE 2g3

| | Position employed in | Relative position (cM) |
|---|---|---|
| Marker on BTA6 | analysis (cM) | http://www.marc.usda.gov/ |
| BM4311 | 89.1 | 97.728 |
| BM2320 | 120.7 | 127.264 |

In one specific embodiment of the present invention, the at least one genetic marker is located in the region from about 81.961 cM to about 127.264 cM (http://www.marc.usda.gov/) on the bovine chromosome BTA6.

In one embodiment the at least one genetic marker is located on the bovine chromosome BTA6 in the region flanked by and including the markers BM415 and BM2320. The at least one genetic marker is selected from the group of markers shown in Table 2g4:

TABLE 2g4

| | Position employed in | Relative position (cM) |
|---|---|---|
| Marker on BTA6 | analysis (cM) | http://www.marc.usda.gov/ |
| BM415 | 76.3 | 81.961 |
| BM4311 | 89.1 | 97.728 |
| BM2320 | 120.7 | 127.264 |

In one specific embodiment of the present invention, the at least one genetic marker is located in the region from about 81.961 cM to about 97.728 cM (http://www.marc.usda.gov/) on the bovine chromosome BTA6.

In one embodiment the at least one genetic marker is located on the bovine chromosome BTA6 in the region flanked by and including the markers BM415 and BM4311. The at least one genetic marker is selected from the group of markers shown in Table 2g5:

TABLE 2g5

| | Position employed in | Relative position (cM) |
|---|---|---|
| Marker on BTA6 | analysis (cM) | http://www.marc.usda.gov/ |
| BM415 | 76.3 | 81.961 |
| BM4311 | 89.1 | 97.728 |

In one specific embodiment of the present invention, the at least one genetic marker is located in the region from about 97.728 cM to about 127.264 cM (http://www.marc.usda.gov/) on the bovine chromosome BTA6.

In one embodiment the at least one genetic marker is located on the bovine chromosome BTA6 in the region flanked by and including the markers BM4311 and BM2320. The at least one genetic marker is selected from the group of markers shown in Table 2g6:

TABLE 2g6

| | Position employed in | Relative position (cM) |
|---|---|---|
| Marker on BTA6 | analysis (cM) | http://www.marc.usda.gov/ |
| BM4311 | 89.1 | 97.728 |
| BM2320 | 120.7 | 127.264 |

In one specific embodiment of the present invention, the at least one genetic marker is located in the region from about 8.053 cM to about 56.12 cM (http://www.marc.usda.gov/) on the bovine chromosome BTA6.

In one embodiment the at least one genetic marker is located on the bovine chromosome BTA6 in the region flanked by and including the markers INRA133 and OARJMP36. The at least one genetic marker is selected from the group of markers shown in Table 2g7:

TABLE 2g7

| | Position employed in | Relative position (cM) |
|---|---|---|
| Marker on BTA6 | analysis (cM) | http://www.marc.usda.gov/ |
| INRA133 | 8.2 | 8.053 |
| BM1329 | 35.5 | 35.398 |
| OARJMP36 | 52.4 | 56.12 |

In one specific embodiment of the present invention, the at least one genetic marker is located in the region from about 35.398 cM to about 81.961 cM (http://www.marc.usda.gov/) on the bovine chromosome BTA6.

In one embodiment the at least one genetic marker is located on the bovine chromosome BTA6 in the region flanked by and including the markers BM1329 and BM415. The at least one genetic marker is selected from the group of markers shown in Table 2g8:

TABLE 2g8

| | Position employed in | Relative position (cM) |
|---|---|---|
| Marker on BTA6 | analysis (cM) | http://www.marc.usda.gov/ |
| BM1329 | 35.5 | 35.398 |
| OARJMP36*¹ | 52.4 | 56.12 |
| BM415 | 76.3 | 81.961 |

In one specific embodiment of the present invention, the at least one genetic marker is located in the region from about 127.264 cM to about 129.985 cM (http://www.marc.usda.gov/) on the bovine chromosome BTA6.

In one embodiment the at least one genetic marker is located on the bovine chromosome BTA6 in the region flanked by and including the markers BM2320 and BL1038. The at least one genetic marker is selected from the group of markers shown in Table 2g9:

TABLE 2g9

| | Position employed in | Relative position (cM) |
|---|---|---|
| Marker on BTA6 | analysis (cM) | http://www.marc.usda.gov/ |
| BM2320 | 120.7 | 127.264 |
| BL1038 | 122.3 | 129.985 |

### BTA7

In yet another aspect of the invention the at least one genetic marker is located on the bovine chromosome BTA7. In one specific embodiment of the present invention, the at least one genetic marker is located in the region from about 0 cM to about 135.564 cM (http://www.marc.usda.gov/) on the bovine chromosome BTA7. In one embodiment the at least one genetic marker is located on the bovine chromosome BTA7 in the region flanked by and including the markers BM7160 and BL1043. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index.

However, in a further embodiment the at least one genetic marker is significant for the traits in any combination. The at least one genetic marker is selected from the group of markers shown in Table 3a:

TABLE 3a

| Markers | Position employed | Relative position (cM) |
|---|---|---|
| on BTA7 | in analysis (cM) | http://www.marc.usda.gov/ |
| BM7160 | 0.0 | 0 |
| BL1067 | 14.2 | 14.683 |
| BMS713 | 15.2 | 16.756 |
| DIK5321 | 22.3 | 22.286 |
| DIK4421 | 22.7 | 22.692 |
| DIK2207 | 26.7 | 26.74 |
| DIK5412 | 30.2 | 30.166 |
| DIK2819 | 47.9 | 47.908 |
| DIK4606 | 55.3 | 55.292 |
| BM7247 | 58.0 | 57.263 |
| UWCA20 | 59.9 | 58.552 |
| BM6117 | 61.0 | 62.246 |
| BMS2840 | 64.3 | 65.305 |
| BMS2258 | 75.0 | 77.194 |
| OARAE129 | 96.6 | 95.93 |
| ILSTS006 | 116.0 | 116.629 |
| BL1043 | 134.1 | 135.564 |

In one embodiment of the present invention, the at least one genetic marker is located in the region from about 55.292 cM to about 77.194 cM (http://www.marc.usda.gov/) on the bovine chromosome BTA7.

In one embodiment the at least one genetic marker is located on the bovine chromosome BTA7 in the region flanked by and including the markers DIK4606 and BMS2258. The at least one genetic marker is selected from the group of markers shown in Table 3b:

TABLE 3b

| Markers | Position employed | Relative position (cM) |
|---|---|---|
| on BTA7 | in analysis (cM) | http://www.marc.usda.gov/ |
| DIK4606 | 55.3 | 55.292 |
| BM7247 | 58.0 | 57.263 |
| UWCA20 | 59.9 | 58.552 |
| BM6117 | 61.0 | 62.246 |
| BMS2840 | 64.3 | 65.305 |
| BMS2258 | 75.0 | 77.194 |

In another preferred embodiment, the at least one genetic marker is located in the region from about 55.292 cM to about 62.246 cM (http://www.marc.usda.gov/) on the bovine chromosome BTA7.

In one embodiment the at least one genetic marker is located on the bovine chromosome BTA7 in the region flanked by and including the markers DIK4606 and BM6117. The at least one genetic marker is selected from the group of markers shown in Table 3b1:

TABLE 3b1

| Markers | Position employed | Relative position (cM) |
|---|---|---|
| on BTA7 | in analysis (cM) | http://www.marc.usda.gov/ |
| DIK4606 | 55.3 | 55.292 |
| BM7247 | 58.0 | 57.263 |
| UWCA20 | 59.9 | 58.552 |
| BM6117 | 61.0 | 62.246 |

In yet another preferred embodiment of the present invention, the at least one genetic marker is located in the region from about 58.552 cM to about 77.194 cM (http://www.marc.usda.gov/) on the bovine chromosome BTA7. In one embodiment the at least one genetic marker is located on the bovine chromosome BTA7 in the region flanked by and including the markers UWCA20 and BMS2258. The at least one genetic marker is selected from the group of markers shown in Table 3b2:

TABLE 3b2

| | Position employed in | Relative position (cM) |
|---|---|---|
| Markers on BTA7 | analysis (cM) | http://www.marc.usda.gov/ |
| UWCA20 | 59.9 | 58.552 |
| BM6117 | 61.0 | 62.246 |
| BMS2840 | 64.3 | 65.305 |
| BMS2258 | 75.0 | 77.194 |

In yet a further preferred embodiment of the present invention, the at least one genetic marker is located in the region from about 57.263 cM to about 65.305 cM (http://www.marc.usda.gov/) on the bovine chromosome BTA7. In one embodiment the at least one genetic marker is located on the bovine chromosome BTA7 in the region flanked by and including the markers BM7247 and BMS2840. The at least one genetic marker is selected from the group of markers shown in Table 3b3:

TABLE 3b3

| | Position employed in | Relative position (cM) |
|---|---|---|
| Markers on BTA7 | analysis (cM) | http://www.marc.usda.gov/ |
| BM7247 | 58.0 | 57.263 |
| UWCA20 | 59.9 | 58.552 |
| BM6117 | 61.0 | 62.246 |
| BMS2840 | 64.3 | 65.305 |

In another embodiment of the present invention, the at least one genetic marker is located in the region from about 95.93 cM to about 116.629 cM (http://www.marc.usda.gov/) on the bovine chromosome BTA7. In one embodiment the at least one genetic marker is located on the bovine chromosome BTA7 in the region flanked by and including the markers OARAE129 and ILSTS006. The at least one genetic marker is selected from the group of markers shown in Table 3c:

TABLE 3c

| | Position employed in | Relative position (cM) |
|---|---|---|
| Markers on BTA7 | analysis (cM) | http://www.marc.usda.gov/ |
| OARAE129 | 96.6 | 95.93 |
| ILSTS006 | 116.0 | 116.629 |

In a further embodiment of the present invention, the at least one genetic marker is located in the region from about 116.629 cM to about 135.564 cM (http://www.marc.usda.gov/) on the bovine chromosome BTA7. In one embodiment the at least one genetic marker is located on the bovine chromosome BTA7 in the region flanked by and including the markers ILSTS006 and BL1043. The at least one genetic marker is selected from the group of markers shown in Table 3d:

TABLE 3d

| | Position employed in | Relative position (cM) |
|---|---|---|
| Markers on BTA7 | analysis (cM) | http://www.marc.usda.gov/ |
| ILSTS006 | 116.0 | 116.629 |
| BL1043 | 134.1 | 135.564 |

In still a further embodiment of the present invention, the at least one genetic marker is located in the region from about 65.305 cM to about 95.93 cM (http://www.marc.usda.gov/) on the bovine chromosome BTA7. In one embodiment the at least one genetic marker is located on the bovine chromosome BTA7 in the region flanked by and including the markers BMS2840 and OARAE129. The at least one genetic marker is selected from the group of markers shown in Table 3e:

TABLE 3e

| | Position employed in | Relative position (cM) |
|---|---|---|
| Markers on BTA7 | analysis (cM) | http://www.marc.usda.gov/ |
| BMS2840 | 64.3 | 65.305 |
| BMS2258 | 75.0 | 77.194 |
| OARAE129 | 96.6 | 95.93 |

In yet a further embodiment of the present invention, the at least one genetic marker is located in the region from about 30.166 cM to about 55.292 cM (http://www.marc.usda.gov/) on the bovine chromosome BTA7. In one embodiment the at least one genetic marker is located on the bovine chromosome BTA7 in the region flanked by and including the markers DIK5412 and DIK4606. The at least one genetic marker is selected from the group of markers shown in Table 3f:

TABLE 3f

| | Position employed in | Relative position (cM) |
|---|---|---|
| Markers on BTA7 | analysis (cM) | http://www.marc.usda.gov/ |
| DIK5412 | 30.2 | 30.166 |
| DIK2819 | 47.9 | 47.908 |
| DIK4606 | 55.3 | 55.292 |

In another embodiment of the present invention, the at least one genetic marker is located in the region from about 95.93 cM to about 135,564 cM (http://www.marc.usda.gov/) on the bovine chromosome BTA7. In one embodiment the at least one genetic marker is located on the bovine chromosome BTA7 in the region flanked by and including the markers OAREA129 and BL1043. The at least one genetic marker is selected from the group of markers shown in Table 3g:

TABLE 3g

| | Position employed in | Relative position (cM) |
|---|---|---|
| Markers on BTA7 | analysis (cM) | http://www.marc.usda.gov/ |
| OARAE129 | 96.6 | 95.93 |
| ILSTS006 | 116.0 | 116.629 |
| BL1043 | 134.1 | 135.564 |

In yet another embodiment of the present invention, the at least one genetic marker is located in the region from about 30,166 cM to about 65,305 cM (http://www.marc.usda.gov/) on the bovine chromosome BTA7. In one embodiment the at least one genetic marker is located on the bovine chromosome BTA7 in the region flanked by and including the markers DIK5412 and BMS2840. The at least one genetic marker is selected from the group of markers shown in Table 3h:

TABLE 3h

| | Position employed in | Relative position (cM) |
|---|---|---|
| Markers on BTA7 | analysis (cM) | http://www.marc.usda.gov/ |
| DIK5412 | 302 | 30.166 |
| DIK2819 | 47.9 | 47.908 |
| DIK4606 | 55.3 | 55.292 |
| BM7247 | 58.0 | 57.263 |
| UWCA20 | 59.9 | 58.552 |
| BM6117 | 61.0 | 62.246 |
| BMS2840 | 64.3 | 65.305 |

### BTA9

In another embodiment of the invention the at least one genetic marker is located on the bovine chromosome BTA9. In one specific embodiment of the present invention, the at least one genetic marker is located in the region from about 4.892 cM to about 109.287 cM (http://www.marc.usda.gov/) on the bovine chromosome BTA9. In one embodiment the at least one genetic marker is located on the bovine chromosome BTA9 in the region flanked by and including the markers BMS2151 and BMS1967. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index.

However, in a further embodiment the at least one genetic marker is significant for the traits in any combination. The at least one genetic marker is selected from the group of markers shown in Table 3i:

TABLE 3i

| | Position employed in | Relative position (cM) |
|---|---|---|
| Marker on BTA9 | analysis (cM) | http://www.marc.usda.gov/ |
| BMS2151 | 0 | 4.892 |
| ETH225*² | 8.1 | 12.754 |
| ILSTS037 | 21 | 26.266 |
| BM2504 | 25.2 | 30.92 |
| BMS1267 | 33.8 | 38.742 |
| UWCA9 | 44.9 | 49.996 |
| BMS1290 | 59.0 | 64.935 |
| BM6436 | 71.1 | 77.554 |
| BMS2753 | 73.1 | 79.249 |
| BMS2819 | 84.4 | 90.98 |
| BM4208 | 84.6 | 90.69 |
| BMS2295 | 91.5 | 98.646 |
| BMS1967 | 102.5 | 109.287 |

*²Also known as MB009

In one specific embodiment of the present invention, the at least one genetic marker is located in the region from about 4.892 cM to about 90.98 cM (http://www.marc.usda.gov/) on the bovine chromosome BTA9.

In one embodiment the at least one genetic marker is located on the bovine chromosome BTA9 in the region flanked by and including the markers BMS2151 and BMS2819. The at least one genetic marker is selected from the group of markers shown in Table 3i1:

TABLE 3i1

| | Position employed in | Relative position (cM) |
|---|---|---|
| Marker on BTA9 | analysis (cM) | http://www.marc.usda.gov/ |
| BMS2151 | 0 | 4.892 |
| ETH225 | 8.1 | 12.754 |
| ILSTS037 | 21 | 26.266 |
| BM2504 | 25.2 | 30.92 |
| BMS1267 | 33.8 | 38.742 |
| UWCA9 | 44.9 | 49.996 |
| BMS1290 | 59.0 | 64.935 |
| BM6436 | 71.1 | 77.554 |
| BMS2753 | 73.1 | 79.249 |
| BMS2819 | 84.4 | 90.98 |

In yet another specific embodiment of the present invention, the at least one genetic marker is located in the region from about 90.69 cM to about 90.98 cM (http://www.marc.usda.gov/) on the bovine chromosome BTA9.

In one embodiment the at least one genetic marker is located on the bovine chromosome BTA9 in the region flanked by and including the markers BM4208 and BMS2819. The at least one genetic marker is selected from the group of markers shown in Table 3i2:

TABLE 3i2

| | Position employed in | Relative position (cM) |
|---|---|---|
| Marker on BTA9 | analysis (cM) | http://www.marc.usda.gov/ |
| BM4208 | 84.6 | 90.69 |
| BMS2819 | 84.4 | 90.98 |

In one specific embodiment of the present invention, the at least one genetic marker is located in the region from about 49.996 cM to about 90.98 cM (http://www.marc.usda.gov/) on the bovine chromosome BTA9.

In one embodiment the at least one genetic marker is located on the bovine chromosome BTA9 in the region flanked by and including the markers UWCA9 and BMS2819. The at least one genetic marker is selected from the group of markers shown in Table 3i3:

TABLE 3i3

| | Position employed in | Relative position (cM) |
|---|---|---|
| Marker on BTA9 | analysis (cM) | http://www.marc.usda.gov/ |
| UWCA9 | 44.9 | 49.996 |
| BMS1290 | 59.0 | 64.935 |
| BM6436 | 71.1 | 77.554 |
| BMS2753 | 73.1 | 79.249 |
| BMS2819 | 84.4 | 90.98 |

In one specific embodiment of the present invention, the at least one genetic marker is located in the region from about 64.935 cM to about 90.69 cM (http://www.marc.usda.gov/) on the bovine chromosome BTA9.

In one embodiment the at least one genetic marker is located on the bovine chromosome BTA9 in the region flanked by and including the markers BMS1290 and BM4208. The at least one genetic marker is selected from the group of markers shown in Table 3i4:

TABLE 3i4

| | Position employed in | Relative position (cM) |
|---|---|---|
| Marker on BTA9 | analysis (cM) | http://www.marc.usda.gov/ |
| BMS1290 | 59.0 | 64.935 |
| BM6436 | 71.1 | 77.554 |
| BMS2753 | 73.1 | 79.249 |
| BMS2819 | 84.4 | 90.98 |
| BM4208 | 84.6 | 90.69 |

In one specific embodiment of the present invention, the at least one genetic marker is located in the region from about 12.754 cM to about 38.742 cM (http://www.marc.usda.gov/) on the bovine chromosome BTA9.

In one embodiment the at least one genetic marker is located on the bovine chromosome BTA9 in the region flanked by and including the markers ETH225 and BMS1267. The at least one genetic marker is selected from the group of markers shown in Table 3i5:

TABLE 3i5

| | Position employed in | Relative position (cM) |
|---|---|---|
| Marker on BTA9 | analysis (cM) | http://www.marc.usda.gov/ |
| ETH225 | 8.1 | 12.754 |
| ILSTS037 | 21 | 26.266 |
| BM2504 | 25.2 | 30.92 |
| BMS1267 | 33.8 | 38.742 |

In one specific embodiment of the present invention, the at least one genetic marker is located in the region from about 12.754 cM to about 26.266 cM (http://www.marc.usda.gov/) on the bovine chromosome BTA9.

In one embodiment the at least one genetic marker is located on the bovine chromosome BTA9 in the region flanked by and including the markers ETH225 and ILSTS037. The at least one genetic marker is selected from the group of markers shown in Table 3i6:

TABLE 3i6

| | Position employed in | Relative position (cM) |
|---|---|---|
| Marker on BTA9 | analysis (cM) | http://www.marc.usda.gov/ |
| ETH225 | 8.1 | 12.754 |
| ILSTS037 | 21 | 26.266 |

In one specific embodiment of the present invention, the at least one genetic marker is located in the region from about 90.98 cM to about 109.287 cM (http://www.marc.usda.gov/) on the bovine chromosome BTA9.

In one embodiment the at least one genetic marker is located on the bovine chromosome BTA9 in the region flanked by and including the markers BMS2819 and BMS1967. The at least one genetic marker is selected from the group of markers shown in Table 3i7:

TABLE 3i7

| | Position employed in | Relative position (cM) |
|---|---|---|
| Marker on BTA9 | analysis (cM) | http://www.marc.usda.gov/ |
| BMS2819 | 84.4 | 90.98 |
| BMS2295 | 91.5 | 98.646 |
| BMS1967 | 102.5 | 109.287 |

In one specific embodiment of the present invention, the at least one genetic marker is located in the region from about 98.646 cM to about 109.287 cM (http://www.marc.usda.gov/) on the bovine chromosome BTA9.

In one embodiment the at least one genetic marker is located on the bovine chromosome BTA9 in the region flanked by and including the markers BMS2285 and BMS1967. The at least one genetic marker is selected from the group of markers shown in Table 3i8:

TABLE 3i8

| | Position employed in | Relative position (cM) |
|---|---|---|
| Marker on BTA9 | analysis (cM) | http://www.marc.usda.gov/ |
| BMS2295 | 91.5 | 98.646 |
| BMS1967 | 102.5 | 109.287 |

In one specific embodiment of the present invention, the at least one genetic marker is located in the region from about 38.742 cM to about 64.935 cM (http://www.marc.usda.gov/) on the bovine chromosome BTA9.

In one embodiment the at least one genetic marker is located on the bovine chromosome BTA9 in the region flanked by and including the markers BMS1267 and BMS1290. The at least one genetic marker is selected from the group of markers shown in Table 3i9:

TABLE 3i9

| | Position employed in | Relative position (cM) |
|---|---|---|
| Marker on BTA9 | analysis (cM) | http://www.marc.usda.gov/ |
| BMS1267 | 33.8 | 38.742 |
| UWCA9 | 44.9 | 49.996 |
| BMS1290 | 59.0 | 64.935 |

In one specific embodiment of the present invention, the at least one genetic marker is located in the region from about 38742 cM to about 49.996 cM (http://www.marc.usda.gov/) on the bovine chromosome BTA9.

In one embodiment the at least one genetic marker is located on the bovine chromosome BTA9 in the region flanked by and including the markers BMS1267 and UWCA9. The at least one genetic marker is selected from the group of markers shown in Table 3i10:

TABLE 3i10

| | Position employed in | Relative position (cM) |
|---|---|---|
| Marker on BTA9 | analysis (cM) | http://www.marc.usda.gov/ |
| BMS1267 | 33.8 | 38.742 |
| UWCA9 | 44.9 | 49.996 |

### BTA11

In another embodiment of the invention the at least one genetic marker is located on the bovine chromosome BTA11. In one specific embodiment of the present invention, the at least one genetic marker is located in the region from about 19.44 cM to about 122.37 cM (http://www.marc.usda.gov/) on the bovine chromosome BTA11. In one embodiment the at least one genetic marker is located on the bovine chromosome BTA11 in the region flanked by and including the markers BM716 and HELL 3. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index.

However, in a further embodiment the at least one genetic marker is significant for the traits in any combination. The at least one genetic marker is selected from the group of markers shown in Table 3j:

TABLE 3j

| | Position employed in | Relative position (cM) |
|---|---|---|
| Marker on BTA11 | analysis (cM) | http://www.marc.usda.gov/ |
| BM716 | 9.5 | 19.44 |
| BMS2569 | 11.7 | 21.082 |
| BM2818 | 20.5 | 30.009 |
| INRA177 2 | 25.7 | 34.802 |
| RM096*³ | 31.3 | 40.481 |
| INRA131 | 38.0 | 47.289 |
| BM7169 | 41.0 | 50.312 |
| BM6445 | 56.9 | 61.57 |
| BMS1822 | 61.2 | 65.879 |
| TGLA58*⁴ | 67.5 | 73.136 |
| BMS2047 | 73.8 | 78.457 |
| HUJV174 | 85.4 | 92.179 |
| TGLA436 | 98.5 | 105.214 |
| HEL13*⁵ | 114.5 | 122.37 |

*³Also known as CA096,
*⁴also known as BMS710,
*⁵also known as MB070.

In one specific embodiment of the present invention, the at least one genetic marker is located in the region from about 78.457 cM to about 122.37 cM (http://www.marc.usda.gov/) on the bovine chromosome BTA11.

In one embodiment the at least one genetic marker is located on the bovine chromosome BTA11 in the region flanked by and including the markers BMS2047 and HELL 3. The at least one genetic marker is selected from the group of markers shown in Table 3j2:

TABLE 3j1

| | Position employed in | Relative position (cM) |
|---|---|---|
| Marker on BTA11 | analysis (cM) | http://www.marc.usda.gov/ |
| BMS2047 | 73.8 | 78.457 |
| HUJV174 | 85.4 | 92.179 |
| TGLA436 | 98.5 | 105.214 |
| HEL13 | 114.5 | 122.37 |

In one specific embodiment of the present invention, the at least one genetic marker is located in the region from about 92.179 cM to about 122.33 cM (http://www.marc.usda.gov/) on the bovine chromosome BTA11.

In one embodiment the at least one genetic marker is located on the bovine chromosome BTA1L1 in the region flanked by and including the markers HUJ174 and HEL13. The at least one genetic marker is selected from the group of markers shown in Table 3j2:

TABLE 3j2

| | Position employed in | Relative position (cM) |
|---|---|---|
| Marker on BTA11 | analysis (cM) | http://www.marc.usda.gov/ |
| HUJV174 | 85.4 | 92.179 |
| TGLA436 | 98.5 | 105.214 |
| HEL13 | 114.5 | 122.37 |

In one specific embodiment of the present invention, the at least one genetic marker is located in the region from about 50.312 cM to about 73.136 cM (http://www.marc.usda.gov/) on the bovine chromosome BTA11.

In one embodiment the at least one genetic marker is located on the bovine chromosome BTA11 in the region flanked by and including the markers BM7169 and TGLA58. The at least one genetic marker is selected from the group of markers shown in Table 3j3:

TABLE 3j3

| | Position employed in | Relative position (cM) |
|---|---|---|
| Marker on BTA11 | analysis (cM) | http://www.marc.usda.gov/ |
| BM7169 | 41.0 | 50.312 |
| BM6445 | 56.9 | 61.57 |
| BMS1822 | 61.2 | 65.879 |
| TGLA58 | 67.5 | 73.136 |

In one specific embodiment of the present invention, the at least one genetic marker is located in the region from about 61.57 cM to about 65.879 cM (http://www.marc.usda.gov/) on the bovine chromosome BTA11.

In one embodiment the at least one genetic marker is located on the bovine chromosome BTA11 in the region flanked by and including the markers BM6445 and BMS1822. The at least one genetic marker is selected from the group of markers shown in Table 3j4:

TABLE 3j4

| | Position employed in | Relative position (cM) |
|---|---|---|
| Marker on BTA11 | analysis (cM) | http://www.marc.usda.gov/ |
| BM6445 | 56.9 | 61.57 |
| BMS1822 | 61.2 | 65.879 |

In one specific embodiment of the present invention, the at least one genetic marker is located in the region from about 21.082 cM to about 47.289 cM (http://www.marc.usda.gov/) on the bovine chromosome BTA 1.

In one embodiment the at least one genetic marker is located on the bovine chromosome BTA11 in the region flanked by and including the markers BMS2569 and INRA131. The at least one genetic marker is selected from the group of markers shown in Table 3j5:

TABLE 3j5

| | Position employed in | Relative position (cM) |
|---|---|---|
| Marker on BTA11 | analysis (cM) | http://www.marc.usda.gov/ |
| BMS2569 | 11.7 | 21.082 |
| BM2818 | 20.5 | 30.009 |
| INRA177 2 | 25.7 | 34.802 |
| RM096 | 31.3 | 40.481 |
| INRA131 | 38.0 | 47.289 |

In one specific embodiment of the present invention, the at least one genetic marker is located in the region from about 30.009 cM to about 47.289 cM (http://www.marc.usda.gov/) on the bovine chromosome BTA11.

In one embodiment the at least one genetic marker is located on the bovine chromosome BTA11 in the region flanked by and including the markers BM2818 and INRA131. The at least one genetic marker is selected from the group of markers shown in Table 3j6:

TABLE 3j6

| | Position employed in | Relative position (cM) |
|---|---|---|
| Marker on BTA11 | analysis (cM) | http://www.marc.usda.gov/ |
| BM2818 | 20.5 | 30.009 |
| INRA177 2 | 25.7 | 34.802 |
| RM096 | 31.3 | 40.481 |
| INRA131 | 38.0 | 47.289 |

### BTA15

In yet another embodiment of the invention the at least one genetic marker is located on the bovine chromosome BTA15. In one specific embodiment of the present invention, the at least one genetic marker is located in the region from about 48.216 cM to about 109.753 cM (http://www.marc.usda.gov/) on the bovine chromosome BTA15.

In one embodiment the at least one genetic marker is located on the bovine chromosome BTA15 in the region flanked by and including the markers BMS2684 and BMS429. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index. However, in a further embodiment the at least one genetic marker is significant for the traits in any combination. The at least one genetic marker is selected from the group of markers shown in Table 4a:

TABLE 4a

| | Position employed in | Relative position (cM) |
|---|---|---|
| Marker on BTA15 | analysis (cM) | http://www.marc.usda.gov/ |
| BMS2684 | 34.9 | 48.216 |
| INRA145 | 51.6 | 67.759 |
| IDVGA-10 | 51.7 | 67.759 |
| ILSTS027 | 66.3 | 83.417 |
| BMS812 | 68.8 | 84.894 |
| BMS2076 | 75.4 | 91.848 |
| BL1095 | 77.8 | 94.775 |
| BMS820 | 81.6 | 98.184 |
| BMS927 | 88.3 | 104.998 |
| BMS429 | 93.4 | 109.753 |

In one particular embodiment of the present invention, the at least one genetic marker is located in the region from about 98.184 cM to about 109.753 cM (http://www.marc.usda.gov/) on the bovine chromosome BTA15. In one embodiment the at least one genetic marker is located on the bovine chromosome BTA15 in the region flanked by and including the markers BMS820 and BMS429. The at least one genetic marker is selected from the group of markers shown in Table 4b:

TABLE 4b

| | Position employed in | Relative position (cM) |
|---|---|---|
| Marker on BTA15 | analysis (cM) | http://www.marc.usda.gov/ |
| BMS820 | 81.6 | 98.184 |
| BMS927 | 88.3 | 104.998 |
| BMS429 | 93.4 | 109.753 |

In another particular embodiment, the at least one genetic marker is located in the region from about 98.184 cM to about 104.998 cM (http://www.marc.usda.gov/) on the bovine chromosome BTA15. In one embodiment the at least one genetic marker is located on the bovine chromosome BTA15 in the region flanked by and including the markers BMS820 and BMS927. The at least one genetic marker is selected from the group of markers shown in Table 4b1:

TABLE 4b1

| | Position employed in | Relative position (cM) |
|---|---|---|
| Marker on BTA15 | analysis (cM) | http://www.marc.usda.gov/ |
| BMS820 | 81.6 | 98.184 |
| BMS927 | 88.3 | 104.998 |

In a further particular embodiment of the present invention, the at least one genetic marker is located in the region from about 104.998 cM to about 109.753 cM (http://www.marc.usda.gov/) on the bovine chromosome BTA15. In one embodiment the at least one genetic marker is located on the bovine chromosome BTA15 in the region flanked by and including the markers BMS927 and BMS429. The at least one genetic marker is selected from the group of markers shown in Table 4b2:

TABLE 4b2

| | Position employed in | Relative position (cM) |
|---|---|---|
| Marker on BTA15 | analysis (cM) | http://www.marc.usda.gov/ |
| BMS927 | 88.3 | 104.998 |
| BMS429 | 93.4 | 109.753 |

In yet a further particular embodiment of the present invention, the at least one genetic marker is located in the region from about 48.216 cM to about 83.417 cM (http://www.marc.usda.gov/) on the bovine chromosome BTA15. In one embodiment the at least one genetic marker is located on the bovine chromosome BTA15 in the region flanked by and including the markers BMS2684 and ILSTS027. The at least one genetic marker is selected from the group of markers shown in Table 4c:

TABLE 4c

| | Position employed in | Relative position (cM) |
|---|---|---|
| Marker on BTA15 | analysis (cM) | http://www.marc.usda.gov/ |
| BMS2684 | 34.9 | 48.216 |
| INRA145 | 51.6 | 67.759 |
| IDVGA-10 | 51.7 | 67.759 |
| ILSTS027 | 66.3 | 83.417 |

In yet another embodiment of the present invention, the at least one genetic marker is located in the region from about 67.759 cM to about 83.417 cM (http://www.marc.usda.gov/) on the bovine chromosome BTA15. In one embodiment the at least one genetic marker is located on the bovine chromosome BTA15 in the region flanked by and including the markers IDVGA-10 and ILSTS027. The at least one genetic marker is selected from the group of markers shown in Table 4c1:

TABLE 4c1

| | Position employed in | Relative position (cM) |
|---|---|---|
| Marker on BTA15 | analysis (cM) | http://www.marc.usda.gov/ |
| IDVGA-10 | 51.7 | 67.759 |
| ILSTS027 | 66.3 | 83.417 |

In one embodiment, the at least one genetic marker is located in the region from about 48.216 cM to about 67.759 cM (http://www.marc.usda.gov/) on the bovine chromosome BTA15. In one embodiment the at least one genetic marker is located on the bovine chromosome BTA15 in the region flanked by and including the markers BMS2684 and IDVGA-10. The at least one genetic marker is selected from the group of markers shown in Table 4d:

TABLE 4d

| | Position employed in | Relative position (cM) |
|---|---|---|
| Marker on BTA15 | analysis (cM) | http://www.marc.usda.gov/ |
| BMS2684 | 34.9 | 48.216 |
| INRA145 | 51.6 | 67.759 |
| IDVGA-10 | 51.7 | 67.759 |

In yet another preferred embodiment, the at least one genetic marker is located in the region from about 48.216 cM to about 67.759 cM (http://www.marc.usda.gov/) on the bovine chromosome BTA15. In one embodiment the at least one genetic marker is located on the bovine chromosome BTA15 in the region flanked by and including the markers BMS2684 and INRA145. The at least one genetic marker is selected from the group of markers shown in Table 4d1:

TABLE 4d1

| | Position employed in | Relative position (cM) |
|---|---|---|
| Marker on BTA15 | analysis (cM) | http://www.marc.usda.gov/ |
| BMS2684 | 34.9 | 48.216 |
| INRA145 | 51.6 | 67.759 |

In another preferred embodiment, the at least one genetic marker is located in the region from about 67.759 cM to about 83.417 cM (http://www.marc.usda.gov/) on the bovine chromosome BTA15. In one embodiment the at least one genetic marker is located on the bovine chromosome BTA15 in the region flanked by and including the markers INRA145 and ILSTS027. The at least one genetic marker is selected from the group of markers shown in Table 4d2:

TABLE 4d2

| | Position employed in | Relative position (cM) |
|---|---|---|
| Marker on BTA15 | analysis (cM) | http://www.marc.usda.gov/ |
| INRA145 | 51.6 | 67.759 |
| IDVGA-10 | 51.7 | 67.759 |
| ILSTS027 | 66.3 | 83.417 |

In still another embodiment of the present invention, the at least one genetic marker is located in the region from about 91.848 cM to about 104.998 cM (http://www.marc.usda.gov/) on the bovine chromosome BTA15. In one embodiment the at least one genetic marker is located on the bovine chromosome BTA15 in the region flanked by and including the markers BMS2076 and BMS927. The at least one genetic marker is selected from the group of markers shown in Table 4e:

TABLE 4e

| | Position employed in | Relative position (cM) |
|---|---|---|
| Marker on BTA15 | analysis (cM) | http://www.marc.usda.gov/ |
| BMS2076 | 75.4 | 91.848 |
| BL1095 | 77.8 | 94.775 |
| BMS820 | 81.6 | 98.184 |
| BMS927 | 88.3 | 104.998 |

### BTA21

In yet a further embodiment of the invention the at least one genetic marker is located on the bovine chromosome BTA21. In one specific embodiment of the present invention the at least one genetic marker is located in the region from about 10.969 cM to about 61.247 cM (http://www.marc.usda.gov/) on the bovine chromosome BTA21. In one embodiment the at least one genetic marker is located on the bovine chromosome BTA21 in the region flanked by and including the markers BMS1117 and BM846. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index.

However, in a further embodiment the at least one genetic marker is significant for the traits in any combination. The at least one genetic marker is selected from the group of markers shown in Table 5a:

TABLE 5a

| | Position employed in | Relative position (cM) |
|---|---|---|
| Markers on BTA 21 | analysis (cM) | http://www.marc.usda.gov/ |
| BMS1117 | 9.9 | 10.969 |
| AGLA233 | 20.4 | 21.202 |
| ILSTS095 | 24.4 | 23.735 |
| BM103 | 30.5 | 29.77 |
| IDVGA-45 | 31.8 | 30.887 |
| INRA103 | 37.7 | 35.898 |
| BMS2815 | 46.1 | 41.714 |
| BM846 | 61.247 | 61.247 |

In a specific embodiment of the present invention, the at least one genetic marker is located in the region from about 23.735 cM to about 35.898 cM (http://www.marc.usda.gov/) on the bovine chromosome BTA21.

In one embodiment the at least one genetic marker is located on the bovine chromosome BTA21 in the region flanked by and including the markers ILSTS095 and INRA103. The at least one genetic marker is selected from the group of markers shown in Table 5b:

TABLE 5b

| | Position employed in | Relative position (cM) |
|---|---|---|
| Markers on BTA 21 | analysis (cM) | http://www.marc.usda.gov/ |
| ILSTS095 | 24.4 | 23.735 |
| BM103 | 30.5 | 29.77 |
| IDVGA-45 | 31.8 | 30.887 |
| INRA103 | 37.7 | 35.898 |

In particularly one embodiment of the present invention, the at least one genetic marker is located in the region from about 23.735 cM to about 30.887 cM (http://www.marc.usda.gov/) on the bovine chromosome BTA21.

In one embodiment the at least one genetic marker is located on the bovine chromosome BTA21 in the region flanked by and including the markers ILSTS095 and IDVGA-45. The at least one genetic marker is selected from the group of markers shown in Table 5b1:

TABLE 5b1

| | Position employed in | Relative position (cM) |
|---|---|---|
| Markers on BTA 21 | analysis (cM) | http://www.marc.usda.gov/ |
| ILSTS095 | 24.4 | 23.735 |
| BM103 | 30.5 | 29.77 |
| IDVGA-45 | 31.8 | 30.887 |

In another particular embodiment of the present invention, the at least one genetic marker is located in the region from about 29.77 cM to about 35.898 cM (http://www.marc.usda.gov/) on the bovine chromosome BTA21.

In one embodiment the at least one genetic marker is located on the bovine chromosome BTA21 in the region flanked by and including the markers BM103 and INRA103. The at least one genetic marker is selected from the group of markers shown in Table 5b2:

TABLE 5b2

| | Position employed in | Relative position (cM) |
|---|---|---|
| Markers on BTA 21 | analysis (cM) | http://www.marc.usda.gov/ |
| BM103 | 30.5 | 29.77 |
| IDVGA-45 | 31.8 | 30.887 |
| INRA103 | 37.7 | 35.898 |

In yet another particular embodiment of the present invention, the at least one genetic marker is located in the region from about 29.77 cM to about 30.887 cM (http://www.marc.usda.gov/) on the bovine chromosome BTA21. In one embodiment the at least one genetic marker is located on the bovine chromosome BTA21 in the region flanked by and including the markers BM103 and IDVGA-45. The at least one genetic marker is selected from the group of markers shown in Table 5b3:

TABLE 5b3

| | Position employed in | Relative position (cM) |
|---|---|---|
| Markers on BTA 21 | analysis (cM) | http://www.marc.usda.gov/ |
| BM103 | 30.5 | 29.77 |
| IDVGA-45 | 31.8 | 30.887 |

The at least one genetic marker is, in another embodiment of the present invention, located in the region from about 30.887 cM to about 41.714 cM (http://www.marc.usda.gov/) on the bovine chromosome BTA21. In one embodiment the at least one genetic marker is located on the bovine chromosome BTA21 in the region flanked by and including the markers IDVGA-45 and BMS2815. The at least one genetic marker is selected from the group of markers shown in Table 5c:

TABLE 5c

| | Position employed in | Relative position (cM) |
|---|---|---|
| Markers on BTA 21 | analysis (cM) | http://www.marc.usda.gov/ |
| IDVGA-45 | 31.8 | 30.887 |
| INRA103 | 37.7 | 35.898 |
| BMS2815 | 46.1 | 41.714 |

In a further embodiment of the present invention, the at least one genetic marker is located in the region from about 35.898 cM to about 61.247 cM (http://www.marc.usda.gov/) on the bovine chromosome BTA21 In one embodiment the at least one genetic marker is located on the bovine chromosome BTA21 in the region flanked by and including the markers INRA103 and BM846. The at least one genetic marker is selected from the group of markers shown in Table 5d:

TABLE 5d

| | Position employed in | Relative position (cM) |
|---|---|---|
| Markers on BTA 21 | analysis (cM) | http://www.marc.usda.gov/ |
| INRA103 | 37.7 | 35.898 |
| BMS2815 | 46.1 | 41.714 |
| BM846 | 61.247 | 61.247 |

In another embodiment of the present invention, the at least one genetic marker is located in the region from about 41,714 cM to about 61.247 cM (http://www.marc.usda.gov/) on the bovine chromosome BTA21 In one embodiment the at least one genetic marker is located on the bovine chromosome BTA21 in the region flanked by and including the markers BMS2815 and BM846. The at least one genetic marker is selected from the group of markers shown in Table 5e:

TABLE 5e

| | Position employed in | Relative position (cM) |
|---|---|---|
| Markers on BTA 21 | analysis (cM) | http://www.marc.usda.gov/ |
| BMS2815 | 46.1 | 41.714 |
| BM846 | 61.247 | 61.247 |

### BTA26

In another embodiment of the invention the at least one genetic marker is located on the bovine chromosome BTA11. In one specific embodiment of the present invention, the at least one genetic marker is located in the region from about 2.839 cM to about 66.763 cM (http://www.marc.usda.gov/) on the bovine chromosome BTA26. In one embodiment the at least one genetic marker is located on the bovine chromosome BTA26 in the region flanked by and including the markers BMS651 and BM7237. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index.

However, in a further embodiment the at least one genetic marker is significant for the traits in any combination. The at least one genetic marker is selected from the group of markers shown in Table 5f:

TABLE 5f

| | Position employed in | Relative position (cM) |
|---|---|---|
| Marker on BTA26 | analysis (cM) | http://www.marc.usda.gov/ |
| BMS651 | 2.5 | 2.839 |
| HEL11*⁶ | 20.7 | 22.862 |
| BMS332 | 27.0 | 31.65 |
| RM026 | 37.3 | 37.635 |
| IDVGA-59 | 50.6 | 53.094 |
| BMS882 | 51.0 | 53.477 |
| BM804 | 59.6 | 60.476 |
| BM9284 | 59.7 | 41.648 |
| BM7237 | 64.3 | 66.763 |

*⁶Also known as MB067

In one specific embodiment of the present invention, the at least one genetic marker is located in the region from about 31.65 cM to about 66.763 cM (http://www.marc.usda.gov/) on the bovine chromosome BTA26.

In one embodiment the at least one genetic marker is located on the bovine chromosome BTA in the region flanked by and including the markers BMS332 and BM7237. The at least one genetic marker is selected from the group of markers shown in Table 5f1:

TABLE 5f1

| | Position employed in | Relative position (cM) |
|---|---|---|
| Marker on BTA26 | analysis (cM) | http://www.marc.usda.gov/ |
| BMS332 | 27.0 | 31.65 |
| RM026 | 37.3 | 37.635 |
| IDVGA-59 | 50.6 | 53.094 |
| BMS882 | 51.0 | 53.477 |
| BM804 | 59.6 | 60.476 |
| BM9284 | 59.7 | 41.648 |
| BM7237 | 64.3 | 66.763 |

In one specific embodiment of the present invention, the at least one genetic marker is located in the region from about 41.648 cM to about 60.476 cM (http://www.marc.usda.gov/) on the bovine chromosome BTA26.

In one embodiment the at least one genetic marker is located on the bovine chromosome BTA in the region flanked by and including the markers BM9284 and BM804. The at least one genetic marker is selected from the group of markers shown in Table 5f2:

TABLE 5f2

| | Position employed in | Relative position (cM) |
|---|---|---|
| Marker on BTA26 | analysis (cM) | http://www.marc.usda.gov/ |
| IDVGA-59 | 50.6 | 53.094 |
| BMS882 | 51.0 | 53.477 |
| BM804 | 59.6 | 60.476 |
| BM9284 | 59.7 | 41.648 |

In one specific embodiment of the present invention, the at least one genetic marker is located in the region from about 53.477 cM to about 60.476 cM (http://www.marc.usda.gov/) on the bovine chromosome BTA26.

In one embodiment the at least one genetic marker is located on the bovine chromosome BTA in the region flanked by and including the markers BMS882 and BM804. The at least one genetic marker is selected from the group of markers shown in Table 5f3:

TABLE 5f3

| | Position employed in | Relative position (cM) |
|---|---|---|
| Marker on BTA26 | analysis (cM) | http://www.marc.usda.gov/ |
| BMS882 | 51.0 | 53.477 |
| BM804 | 59.6 | 60.476 |

In one specific embodiment of the present invention, the at least one genetic marker is located in the region from about 53.577 cM to about 66.763 cM (http://www.marc.usda.gov/) on the bovine chromosome BTA26.

In one embodiment the at least one genetic marker is located on the bovine chromosome BTA in the region flanked by and including the markers BMS882 and BM7237. The at least one genetic marker is selected from the group of markers shown in Table 5f4:

TABLE 5f4

| | Position employed in | Relative position (cM) |
|---|---|---|
| Marker on BTA26 | analysis (cM) | http://www.marc.usda.gov/ |
| BMS882 | 51.0 | 53.477 |
| BM804 | 59.6 | 60.476 |
| BM7237 | 64.3 | 66.763 |

In one specific embodiment of the present invention, the at least one genetic marker is located in the region from about 31.65 cM to about 41.648 cM (http://www.marc.usda.gov/) on the bovine chromosome BTA26.

In one embodiment the at least one genetic marker is located on the bovine chromosome BTA in the region flanked by and including the markers BMS332 and BM9284. The at least one genetic marker is selected from the group of markers shown in Table 5f5:

TABLE 5f5

| | Position employed in | Relative position (cM) |
|---|---|---|
| Marker on BTA26 | analysis (cM) | http://www.marc.usda.gov/ |
| BMS332 | 27.0 | 31.65 |
| RM026 | 37.3 | 37.635 |
| BM9284 | 59.7 | 41.648 |

In one specific embodiment of the present invention, the at least one genetic marker is located in the region from about 37.635 cM to about 41.648 cM (http://www.marc.usda.gov/) on the bovine chromosome BTA26.

In one embodiment the at least one genetic marker is located on the bovine chromosome BTA in the region flanked by and including the markers RM026 and BM9284. The at least one genetic marker is selected from the group of markers shown in Table 5f6:

TABLE 5f6

| | Position employed in | Relative position (cM) |
|---|---|---|
| Marker on BTA26 | analysis (cM) | http://www.marc.usda.gov/ |
| RM026 | 37.3 | 37.635 |
| BM9284 | 59.7 | 41.648 |

In one specific embodiment of the present invention, the at least one genetic marker is located in the region from about 41.648 cM to about 53.477 cM (http://www.marc.usda.gov/) on the bovine chromosome BTA26.

In one embodiment the at least one genetic marker is located on the bovine chromosome BTA in the region flanked by and including the markers BM9284 and BMS882. The at least one genetic marker is selected from the group of markers shown in Table 5f7:

TABLE 5f7

| | Position employed in | Relative position (cM) |
|---|---|---|
| Marker on BTA26 | analysis (cM) | http://www.marc.usda.gov/ |
| IDVGA-59 | 50.6 | 53.094 |
| BMS882 | 51.0 | 53.477 |
| BM9284 | 59.7 | 41.648 |

In one specific embodiment of the present invention, the at least one genetic marker is located in the region from about 37.635 cM to about 41.648 cM (http://www.marc.usda.gov/) on the bovine chromosome BTA26.

In one embodiment the at least one genetic marker is located on the bovine chromosome BTA in the region flanked by and including the markers RM026 and BM9284. The at least one genetic marker is selected from the group of markers shown in Table 5f8:

TABLE 5f8

| | Position employed in | Relative position (cM) |
|---|---|---|
| Marker on BTA26 | analysis (cM) | http://www.marc.usda.gov/ |
| RM026 | 37.3 | 37.635 |
| BM9284 | 59.7 | 41.648 |

In one specific embodiment of the present invention, the at least one genetic marker is located in the region from about 41.648 cM to about 53.094 cM (http://www.marc.usda.gov/) on the bovine chromosome BTA26.

In one embodiment the at least one genetic marker is located on the bovine chromosome BTA in the region flanked by and including the markers BM9284 and IDVGA-59. The at least one genetic marker is selected from the group of markers shown in Table 5f9:

TABLE 5f9

| | Position employed in | Relative position (cM) |
|---|---|---|
| Marker on BTA26 | analysis (cM) | http://www.marc.usda.gov/ |
| IDVGA-59 | 50.6 | 53.094 |
| BM9284 | 59.7 | 41.648 |

In one specific embodiment of the present invention, the at least one genetic marker is located at the 41.648 cM position (http://www.marc.usda.gov/) on the bovine chromosome BTA26.

In one embodiment the at least one genetic marker is located on the bovine chromosome BTA in the region comprising the marker BM9284. The at least one genetic marker is selected from the group of markers shown in Table 5f10:

TABLE 5f10

| | Position employed in | Relative position (cM) |
|---|---|---|
| Marker on BTA11 | analysis (cM) | http://www.marc.usda.gov/ |
| BM9284 | 59.7 | 41.648 |

### BTA27

On the bovine chromosome BTA27, in yet a further embodiment of the invention, is located the at least one genetic marker. In one specific embodiment of the present invention, the at least one genetic marker is located in the region from about 5.389 cM to about 64.098 cM (http://www.marc.usda.gov/) on the bovine chromosome BTA27. In one embodiment the at least one genetic marker is located on the bovine chromosome BTA27 in the region flanked by and including the markers BMS1001 and BM203. The at least one genetic marker is significant for the traits CELL, MAS1, MAS2, MAS3, MAS4 and/or udder health. In a particular embodiment the at least one genetic marker is significant for example the trait MAS1, such as MAS2, for example MAS3, such as MAS4, for example udder health index. However, in a further embodiment the at least one genetic marker is significant for the traits in any combination. The at least one genetic marker is selected from the group of markers shown in Table 6a:

TABLE 6a

| | Position employed in | Relative position (cM) |
|---|---|---|
| Markers on BTA 27 | analysis (M) | http://www.marc.usda.gov/ |
| BMS1001 | 0.054 | 5.389 |
| BMS 2650 | 0.123 | 12.285 |
| INRA016 | 0.172 | 17.186 |
| BMS2137 | 0.208 | 20.781 |
| CSSM043 | 0.345 | 34.525 |
| IOBT313 | 0.345 | 34.525 |
| INRA134 | 0.453 | 45.253 |
| BM1857 | 0.523 | 52.326 |
| BMS2116 | 0.544 | 54.389 |
| HUJI-13 | 0.557 | 55.75 |
| BM203 | 0.641 | 64.098 |

In a specific embodiment of the present invention, the at least one genetic marker is located in the region from about 45.253 cM to about 52.326 cM (http://www.marc.usda.gov/) on the bovine chromosome BTA27. In one embodiment the at least one genetic marker is located on the bovine chromosome BTA27 in the region flanked by and including the markers INRA134 and BM1857. The at least one genetic marker is selected from the group of markers shown in Table 6b:

TABLE 6b

| | Position employed in | Relative position (cM) |
|---|---|---|
| Markers on BTA 27 | analysis (M) | http://www.marc.usda.gov/ |
| INRA134 | 0.453 | 45.253 |
| BM1857 | 0.523 | 52.326 |

In another specific embodiment of the present invention, the at least one genetic marker is located in the region from about 55.75 cM to about 64.098 cM (http://www.marc.usda.gov/) on the bovine chromosome BTA27.

In one embodiment the at least one genetic marker is located on the bovine chromosome BTA27 in the region flanked by and including the markers HUJI-13 and BM203. The at least one genetic marker is selected from the group of markers shown in Table 6c:

TABLE 6c

| | Position employed in | Relative position (cM) |
|---|---|---|
| Markers on BTA 27 | analysis (M) | http://www.marc.usda.gov/ |
| HUJI-13 | 0.557 | 55.75 |
| BM203 | 0.641 | 64.098 |

In yet another specific embodiment of the present invention, the at least one genetic marker is located in the region from about 54.389 cM to about 55.75 cM (http://www.marc.usda.gov/) on the bovine chromosome BTA27.

In one embodiment the at least one genetic marker is located on the bovine chromosome BTA27 in the region flanked by and including the markers BM2116 and HUJI-13. The at least one genetic marker is selected from the group of markers shown in Table 6d:

TABLE 6d

| | Position employed in | Relative position (cM) |
|---|---|---|
| Markers on BTA 27 | analysis (M) | http://www.marc.usda.gov/ |
| BMS2116 | 0.544 | 54.389 |
| HUJI-13 | 0.557 | 55.75 |

In a further embodiment of the present invention, the at least one genetic marker is located in the region from about 34.525 cM to about 45.253 cM (http://www.marc.usda.gov/) on the bovine chromosome BTA27. In one embodiment the at least one genetic marker is located on the bovine chromosome BTA27 in the region flanked by and including the markers CSSM043 and INRA134. The at least one genetic marker is selected from the group of markers shown in Table 6e:

TABLE 6e

| | Position employed in | Relative position (cM) |
|---|---|---|
| Markers on BTA 27 | analysis (M) | http://www.marc.usda.gov/ |
| CSSM043 | 0.345 | 34.525 |
| IOBT313 | 0.345 | 34.525 |
| INRA134 | 0.453 | 45.253 |

In yet another embodiment of the present invention, the at least one genetic marker is located in the region from about 52.326 cM to about 54.389 cM (http://www.marc.usda.gov/) on the bovine chromosome BTA27 In one embodiment the at least one genetic marker is located on the bovine chromosome BTA27 in the region flanked by and including the markers BM1857 and BMS2116. The at least one genetic marker is selected from the group of markers shown in Table 6f:

TABLE 6f

| | Position employed in | Relative position (cM) |
|---|---|---|
| Markers on BTA 27 | analysis (M) | http://www.marc.usda.gov/ |
| BM1857 | 0.523 | 52.326 |
| BMS2116 | 0.544 | 54.389 |

In a further preferred embodiment of the present invention, the at least one genetic marker is located in the region from about 20.781 cM to about 34.525 cM (http://www.marc.usda.gov/) on the bovine chromosome BTA27. In one embodiment the at least one genetic marker is located on the bovine chromosome BTA27 in the region flanked by and including the markers BMS2137 and CSSM043. The at least one genetic marker is selected from the group of markers shown in Table 6g:

TABLE 6g

| | Position employed in | Relative position (cM) |
|---|---|---|
| Markers on BTA 27 | analysis (cM) | http://www.marc.usda.gov/ |
| BMS2137 | 0.208 | 20.781 |
| CSSM043 | 0.345 | 34.525 |

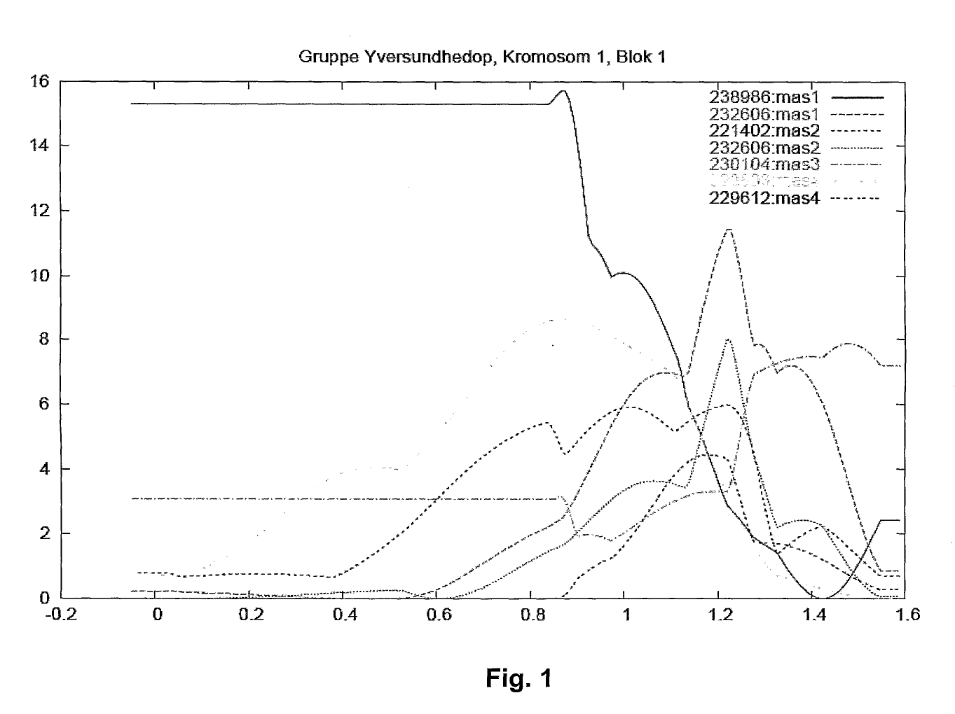

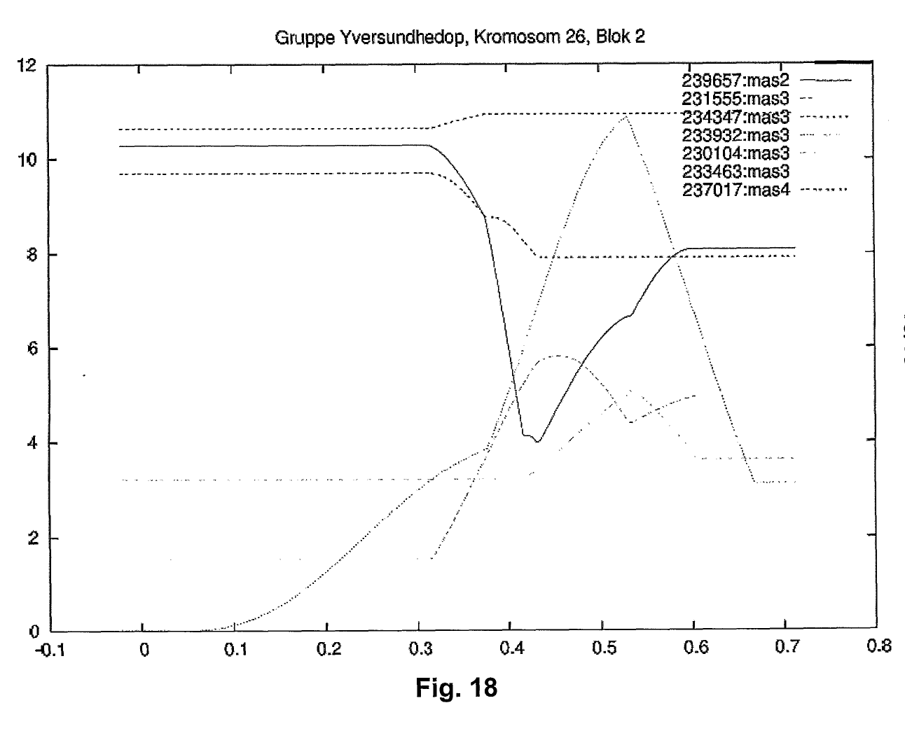

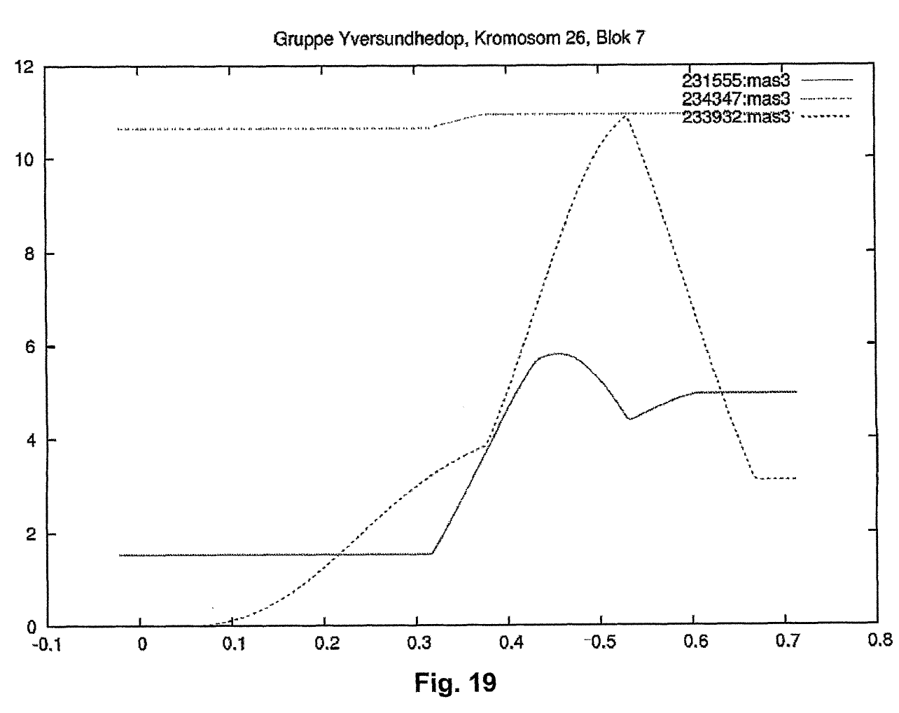

The region of the bovine chromosomes, comprising the genetic markers useful in the present invention is shown in FIGS. 1-19.

In another embodiment of the present invention, the at least one genetic marker is a combination of markers, as indicated in tables 6h1 to 6h10. It is understood that the term BTA1, BTA5. BTA6, BTA7, BTA9, BTA11, BTA15, BTA21, BTA26, BTA27 in tables 6h1 to 6h10 is meant to comprise any regions and genetic markers located on the bovine chromosomes, respectively, as described elsewhere herein.

The tables 6h1 to 6h10 show different embodiments, wherein the combination of markers is a multiplicity of bovine chromosomes, wherein the specific chromosome in each embodiment is indicated with X.

TABLE 6h1

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | X | X | | | | | | | | |
| 2 | X | | X | | | | | | | |
| 3 | X | | | X | | | | | | |
| 4 | X | | | | X | | | | | |
| 5 | X | | | | | X | | | | |
| 6 | X | | | | | | X | | | |
| 7 | X | | | | | | | X | | |
| 8 | X | | | | | | | | X | |
| 9 | X | | | | | | | | | X |
| 10 | X | X | | | X | X | | | | |
| 11 | X | X | | | X | | | | | |
| 12 | X | | | | | X | | | | |
| 13 | X | | | | X | X | | | | |
| 14 | X | | X | | | | X | | X | |
| 15 | X | | X | | | | | X | | |
| 16 | X | | | | | X | | X | | |
| 17 | X | | | | X | | | X | | |
| 18 | X | X | | | | | | X | | |
| 19 | X | X | X | X | X | X | X | X | X | X |

TABLE 6h2

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | | X | X | | | | | | | |
| 2 | | X | | X | | | | | | |
| 3 | | X | | | X | | | | | |
| 4 | | X | | | | X | | | | |
| 5 | | X | | | | | X | | | |
| 6 | | X | | | | | | X | | |
| 7 | | X | | | | | | | X | |
| 8 | | X | | | | | | | | X |
| 9 | | X | | | X | X | | | | |
| 10 | | X | | | X | | | | | |
| 11 | | X | | | | X | | | | |
| 12 | | X | | | | X | | | | |
| 13 | X | X | X | | | | X | | X | |
| 14 | | X | X | | | | | X | | |
| 15 | X | X | | | | X | | X | | |
| 16 | X | X | | | X | | | X | | |
| 17 | | X | | | | | | X | | |
| 18 | | X | X | X | X | X | X | X | X | X |

TABLE 6h3

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | | | X | X | | | | | | |
| 2 | | | X | | X | | | | | |
| 3 | | | X | | | X | | | | |
| 4 | | | X | | | | X | | | |
| 5 | | | X | | | | | X | | |
| 6 | | | X | | | | | | X | |
| 7 | | | X | | | | | | | X |
| 8 | | | X | | X | X | | | | |
| 9 | | | X | | X | | | | | |
| 10 | | | X | | | X | | | | |
| 11 | X | | X | | X | X | | | | |
| 12 | X | | X | | | | X | | X | |
| 13 | X | | X | | | | | X | | |
| 14 | | | X | | | X | | X | | |
| 15 | | | X | | X | | | X | | |
| 16 | X | | X | X | X | X | X | X | X | X |

TABLE 6h4

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | | | | X | X | | | | | |
| 2 | | | | X | | X | | | | |
| 3 | | | | X | | | X | | | |
| 4 | | | | X | | | | X | | |
| 5 | | | | X | | | | | X | |
| 6 | | | | X | | | | | | X |
| 7 | | X | | X | X | X | | | | |
| 8 | | X | | X | X | | | | | |
| 9 | | X | | X | | X | | | | |
| 10 | | | | X | X | X | | | | |
| 11 | | | X | X | | | X | | X | |
| 12 | | | X | X | | | | X | | |
| 13 | X | | | X | | X | | X | | |
| 14 | | | | X | X | | | X | | |
| 15 | | X | | X | | | | X | | |
| 16 | | X | | X | X | X | X | X | X | X |

TABLE 6h5

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | | | | | X | X | | | | |
| 2 | | | | | X | | X | | | |
| 3 | | | | | X | | | X | | |
| 4 | | | | | X | | | | X | |
| 5 | | | | | X | | | | | X |
| 6 | | X | | | X | X | | | | |
| 7 | | X | | | X | | | | | |
| 8 | | | | | X | X | | | | |
| 9 | | | | | X | X | | | | |
| 10 | | | X | | X | | X | | X | |
| 11 | | | X | | X | | | X | | |
| 12 | | | | | X | X | | X | | |
| 13 | X | | | | X | | | X | | |
| 14 | | X | | | X | | | X | | |
| 15 | X | | | | X | | | | | |
| 16 | | X | | X | X | | | | | |
| 17 | | | | | X | | X | X | | X |
| 18 | | X | | X | X | | X | X | X | X |

TABLE 6h6

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | | | | | | X | X | | | |
| 2 | | | | | | X | | X | | |
| 3 | | | | | | X | | | X | |
| 4 | | | | | | X | | | | X |
| 5 | | X | | | X | X | | | | |
| 6 | | X | | | | X | | | | |
| 7 | | | | | | X | | | | |
| 8 | | | | | X | X | | | | |
| 9 | X | | X | | | X | X | | X | |
| 10 | | | X | | | X | | X | | |
| 11 | X | X | | | | X | | X | | |
| 12 | | | | | X | X | | X | | |
| 13 | | X | | | | X | | X | | |
| 14 | X | | X | | X | X | X | | | |
| 15 | | X | X | | | X | | X | | |
| 16 | | | | | | X | | X | | |
| 17 | | | X | X | X | X | | X | X | |

TABLE 6h7

| Embodiment | BTA1 | BTA5 | BTA6 | BTA7 | BTA9 | BTA11 | BTA15 | BTA21 | BTA26 | BTA27 |
|---|---|---|---|---|---|---|---|---|---|---|
| 1 | | | | | | | X | X | | |
| 2 | | | | | | | X | | X | |
| 3 | | | | | | | X | | | X |
| 4 | | X | | | X | X | X | | | |
| 5 | | X | | | X | | X | | | |
| 6 | | X | | | | X | X | | | |
| 7 | | | | | X | X | X | | | |
| 8 | | | X | | | | X | | X | |
| 9 | | | X | | | | X | X | | |
| 10 | | | | | | X | X | X | | |
| 11 | X | X | | | X | X | X | X | | |
| 12 | | X | | | | | X | X | | |
| 13 | | | | | | X | X | X | | |
| 14 | X | | | | X | | X | | | |
| 15 | X | | | | | | X | | | |
| 16 | | | | X | | | X | | X | |

TABLE 6h8

| | | | | | | BTA | BTA | BTA | BTA | BTA |
|---|---|---|---|---|---|---|---|---|---|---|
| Embodiment | BTA 1 | BTA 5 | BTA 6 | BTA 7 | BTA 9 | 11 | 15 | 21 | 26 | 27 |
| 1 | | | | | | | | X | X | |
| 2 | | | | | | | | X | | X |
| 3 | | X | | | X | X | | X | | |
| 4 | | X | | | X | | | X | | |
| 5 | | X | | | | X | | X | | |
| 6 | | | | | | X | | X | | |
| 7 | | | | | X | | | X | | |
| 8 | | X | | | | | | X | | |
| 9 | | | | | | X | | X | | |
| 10 | | | | | X | | | X | | |
| 11 | | X | | | | | | X | | |
| 12 | | | | | | | | X | X | |
| 13 | | X | | X | | X | X | X | | X |
| 14 | | | | | | | | X | | |
| 15 | X | X | | | X | X | | X | | |
| 16 | X | X | X | | X | X | | X | X | |

TABLE 6h9

| | | | | | | BTA | BTA | BTA | BTA | BTA |
|---|---|---|---|---|---|---|---|---|---|---|
| Embodiment | BTA 1 | BTA 5 | BTA 6 | BTA 7 | BTA 9 | 11 | 15 | 21 | 26 | 27 |
| 1 | | | | | | | | | X | X |
| 2 | | X | | | X | X | | | X | |
| 3 | | X | | | X | | | | X | |
| 4 | | X | | | | X | | | X | |
| 5 | | | | | X | X | | | X | |
| 6 | | | | | X | | | | X | |
| 7 | | | | | | X | | | X | |
| 8 | | | | | | X | | X | X | |
| 9 | | | | | X | | | X | X | |
| 10 | | X | | | | | | X | X | |
| 11 | X | | | | | | | | X | |
| 12 | X | X | | | X | X | | X | X | |
| 13 | | X | | X | | | X | | X | X |
| 14 | X | | X | | X | X | | | X | |
| 15 | | X | | X | | | X | | X | X |
| 16 | X | | X | | | X | | X | X | X |

TABLE 6h10

| | | | | | | BTA | BTA | BTA | BTA | BTA |
|---|---|---|---|---|---|---|---|---|---|---|
| Embodiment | BTA 1 | BTA 5 | BTA 6 | BTA 7 | BTA 9 | 11 | 15 | 21 | 26 | 27 |
| 1 | | X | | | X | X | | | | X |
| 2 | | X | | | X | | | | | X |
| 3 | | X | | | | X | | | | X |
| 4 | | | | | X | X | | | | X |
| 5 | | | | | X | | | | | X |
| 6 | | | | | | X | | | | X |
| 7 | X | X | | | X | X | | X | | X |
| 8 | X | | | | X | | | X | | X |
| 9 | | X | | | | X | | X | | X |
| 10 | | X | X | | X | X | | | | X |
| 11 | | X | | | X | | | | X | X |
| 12 | | | | | | X | | | | X |
| 13 | X | | | X | X | X | | X | | X |
| 14 | | | X | | | | X | | X | X |
| 15 | | | X | | | | | X | | X |

### Detection

The detection of the presence or absence of a genetic marker according to the present invention may be conducted on the DNA sequence of the bovine chromosomes BTA1, BTA5, BTA6, BTA9, BTA11, BTA15, BTA21, BTA7 and/or BTA27 specified elsewhere herein according to the present invention or a complementary sequence as well as on transcriptional (mRNA) and translational products (polypeptides, proteins) therefrom.

It will be apparent to the person skilled in the art that there are a large number of analytical procedures which may be used to detect the presence or absence of variant nucleotides at one or more of positions mentioned herein in the specified region. Mutations or polymorphisms within or flanking the specified region can be detected by utilizing a number of techniques. Nucleic acid from any nucleated cell can be used as the starting point for such assay techniques, and may be isolated according to standard nucleic acid preparation procedures that are well known to those of skill in the art. In general, the detection of allelic variation requires a mutation discrimination technique, optionally an amplification reaction and a signal generation system.

A number of mutation detection techniques are listed in Table 7. Some of the methods listed in Table 7 are based on the polymerase chain reaction (PCR), wherein the method according to the present invention includes a step for amplification of the nucleotide sequence of interest in the presence of primers based on the nucleotide sequence of the variable nucleotide sequence. The methods may be used in combination with a number of signal generation systems, a selection of which is also listed in Table 7.

TABLE 7

| | |
|---|---|
| General | DNA sequencing, Sequencing by hybridisation, |
| techniques | SNAPshot |
| Scanning | Single-strand conformation polymorphism analysis, |
| techniques | Denaturing gradient gel electrophoresis, Temperature |
| | gradient gel electrophoresis, Chemical mismatch |
| | cleavage, cleavage, heteroduplex analysis, enzymatic |
| | mismatch cleavage |
| Hybridisation | Solid phase hybridisation: Dot blots, Multiple allele |
| based | specific diagnostic assay (MASDA), Reverse dot blots, |
| techniques | Oligonucleotide arrays (DNA Chips) |
| | Solution phase hybridisation: Taqman - U.S. Pat. No. |
| | 5,210,015 & 5,487,972 (Hoffmann-La Roche), |
| | Molecular Beacons -- Tyagi et al (1996), Nature |
| | Biotechnology, 14, 303; WO 95/13399 (Public |
| | Health Inst., New York), Lightcycler, optionally in |
| | combination with Fluorescence resonance energy |
| | transfer (FRET). |
| Extension based | Amplification refractory mutation system (ARMS), |
| techniques | Amplification refractory mutation system linear |
| | extension (ALEX) - European Patent No. EP 332435 |
| | B1 (Zeneca Limited), Competitive oligonucleotide |
| | priming system (COPS) - Gibbs et al (1989), |
| | Nucleic Acids Research, 17, 2347. |
| Incorporation | Mini-sequencing, Arrayed primer extension (APEX) |
| based techniques | |
| Restriction | Restriction fragment length polymorphism (RFLP), |
| Enzyme | Restriction site generating PCR |
| based techniques | |
| Ligation based | Oligonucleotide ligation assay (OLA) |
| techniques | |
| Other | Invader assay |
| Various Signal | Fluorescence: |
| Generation or | Fluorescence resonance energy transfer (FRET), |
| Detection | Fluorescence quenching, Fluorescence polarisation-- |
| Systems | United Kingdom Patent No. 2228998 (Zeneca Limited) |
| Other | Chemiluminescence, Electrochemiluminescence, |
| | Raman, Radioactivity, Colorimetric, Hybridisation |
| | protection assay, Mass spectrometry |

Further amplification techniques are listed in Table 8. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997. The detection of genetic markers can according to one embodiment of the present invention be achieved by a number of techniques known to the skilled person, including typing of microsatellites or short tandem repeats (STR), restriction fragment length polymorphisms (RFLP), detection of deletions or insertions, random amplified polymorphic DNA (RAPIDs) or the typing of single nucleotide polymorphisms by methods such as restriction fragment length polymerase chain reaction, allele-specific oligomer hybridisation, oligomer-specific ligation assays, hybridisation with PNA or locked nucleic acids (LNA) probes.

TABLE 8

| | |
|---|---|
| Further amplification | Self sustained replication (SSR), |
| techniques | Nucleic acid sequence based |
| | amplification (NASBA), |
| | Ligase chain reaction (LCR), |
| | Strand displacement amplification (SDA) |

A primer of the present invention is a nucleic acid molecule sufficiently complementary to the sequence on which it is based and of sufficiently length to selectively hybridise to the corresponding region of a nucleic acid molecule intended to be amplified. The primer is able to prime the synthesis of the corresponding region of the intended nucleic acid molecule in the methods described above. Similarly, a probe of the present invention is a molecule for example a nucleic acid molecule of sufficient length and sufficiently complementary to the nucleic acid sequence of interest which selectively binds to the nucleic acid sequence of interest under high or low stringency conditions.

### Sample

The method according to the present invention includes analyzing a sample of a bovine subject, wherein said sample may be any suitable sample capable of providing the bovine genetic material for use in the method. The bovine genetic material may for example be extracted, isolated and purified if necessary from a blood sample, a tissue samples (for example spleen, buccal smears), clipping of a body surface (hairs or nails), milk and/or semen. The samples may be fresh or frozen.

The DNA polymorphisms of the invention comprise at least one nucleotide difference, such as at least two nucleotide differences, for example at least three nucleotide differences, such as at least four nucleotide differences, for example at least five nucleotide differences, such as at least six nucleotide differences, for example at least seven nucleotide differences, such as at least eight nucleotide differences, for example at least nine nucleotide differences, such as 10 nucleotide differences. The nucleotide differences comprise nucleotide differences, deletion and/or insertion or any combination thereof.

### Primers

The primers that may be used according to the present invention are shown in Table 9. The in Table 9 specified primer pairs may be used individually or in combination with one or more primer pairs of Table 9.

The design of such primers or probes will be apparent to the molecular biologist of ordinary skill. Such primers are of any convenient length such as up to 50 bases, up to 40 bases, more conveniently up to 30 bases in length, such as for example 8-25 or 8-15 bases in length. In general such primers will comprise base sequences entirely complementary to the corresponding wild type or variant locus in the region. However, if required one or more mismatches may be introduced, provided that the discriminatory power of the oligonucleotide probe is not unduly affected. The primers/probes of the invention may carry one or more labels to facilitate detection.

In one embodiment, the primers and/or probes are capable of hybridizing to and/or amplifying a subsequence hybridizing to a single nucleotide polymorphism containing the sequence delineated by the markers as shown herein.

The primer nucleotide sequences of the invention further include: (a) any nucleotide sequence that hybridizes to a nucleic acid molecule of the delineated region(s) or its complementary sequence or RNA products under stringent conditions, e.g., hybridization to filter-bound DNA in 6× sodium chloride/sodium citrate (SSC) at about 45° C. followed by one or more washes in 0.2×SSC/0.1% Sodium Dodecyl Sulfate (SDS) at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the nucleic acid molecule that hybridizes to the nucleotide sequence of (a) and (b), above, is one that comprises the complement of a nucleic acid molecule of the region s or r or a complementary sequence or RNA product thereof. In a preferred embodiment, nucleic acid molecules comprising the nucleotide sequences of (a) and (b), comprises nucleic acid molecule of RAI or a complementary sequence or RNA product thereof.

Among the nucleic acid molecules of the invention are deoxyoligonucleotides (“oligos”) which hybridize under highly stringent or stringent conditions to the nucleic acid molecules described above. In general, for probes between 14 and 70 nucleotides in length the melting temperature (TM) is calculated using the formula:

Tm(° C.)=81.5+16.6(log[monovalent cations(molar)])+0.41(% G+C)−(500/N)

where N is the length of the probe. If the hybridization is carried out in a solution containing formamide, the melting temperature is calculated using the equation Tm(° C.)=81.5+16.6(log[monovalent cations (molar)])+0.41 (% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer for example to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos). Accordingly, the invention further provides nucleotide primers or probes which detect the r region polymorphisms of the invention. The assessment may be conducted by means of at least one nucleic acid primer or probe, such as a primer or probe of DNA, RNA or a nucleic acid analogue such as peptide nucleic acid (PNA) or locked nucleic acid (LNA).

According to one aspect of the present invention there is provided an allele-specific oligonucleotide probe capable of detecting a polymorphism at one or more of positions in the delineated regions 1.

The allele-specific oligonucleotide probe is preferably 5-50 nucleotides, more preferably about 5-35 nucleotides, more preferably about 5-30 nucleotides, more preferably at least 9 nucleotides.

### Determination of Linkage

In order to detect whether the genetic marker is present in the genetic material, standard methods well known to persons skilled in the art may be applied, for example by the use of nucleic acid amplification. In order to determine whether the genetic marker is genetically linked to the udder health traits, a permutation test can be applied when the regression method is used (Doerge and Churchill, 1996), or the Piepho-method can be applied (Piepho, 2001) when the variance components method is used. The principle of the permutation test is well described by Doerge and Churchill (1996), whereas the Piepho-method is well described by Piepho (2001). Significant linkage in the within family analysis using the regression method, a 1000 permutations were made using the permutation test (Doerge and Churchill, 1996). A threshold at the 5% chromosome wide level was considered to be significant evidence for linkage between the genetic marker and the udder health traits. In addition, the QTL was confirmed in different sire families. For the across family analysis and multi-trait analysis with the variance component method the piepho method was used to determine the significance level (Piepho, 2001). A threshold at the 5% chromosome wide level was considered to be significant evidence for linkage between the genetic marker and the udder health traits.

### Kit

Another aspect of the present invention relates to A diagnostic kit for use in detecting the presence or absence in a bovine subject of at least one genetic marker associated with bovine udder health, comprising at least one oligonucleotide sequence and combinations thereof, wherein the nucleotide sequences are selected from any of SEQ ID NO.: 1 to SEQ ID NO.:206 and/or any combination thereof.

Genotyping of a bovine subject in order to establish the genetic determinants of udder health for that subject according to the present invention can be based on the analysis of genomic DNA which can be provided using standard DNA extraction methods as described herein. The genomic DNA may be isolated and amplified using standard techniques such as the polymerase chain reaction using oligonucleotide primers corresponding (complementary) to the polymorphic marker regions. Additional steps of purifying the DNA prior to amplification reaction may be included. Thus, a diagnostic kit for establishing udder health characteristics comprises, in a separate packing, at least one oligonucleotide sequence selected from the group of sequences shown in table 9 and any combinations thereof.

### EXAMPLES

### Animals

The animal material used in example 1-10 consists of a granddaughter design with 19 paternal Danish Holstein sire families with a total 1,373 offspring tested sons. The number of sons per grandsire ranged from 33 to 105, with an average family size of 72.3.

### Purification of Genomic DNA

Genomic DNA was purified from semen according to the following protocol:

After thawing the semen-straw, both ends of the straw were cut away with a pair of scissors and the content of semen transferred to a 1.5 ml eppendorf tube. 1 ml of 0.9% NaCl was used to flush the straw into the tube. The tube was then centrifuged for 5 minutes at 2000 rpm, followed by removal of the supernatant. This washing step was repeated twice.

Then 3001 buffer S (10 mM Tris HCl pH 8, 100 mM NaCl, 10 mM EDTA pH 8; 0.5% SDS), 20 μl 1 M DTT and 20 μl pronase (20 mg/ml) (Boehringer) are added to the tube. After mixing the tubes are incubated over night with slow rotation where after 180 μl saturated NaCl is added followed by vigorous agitation for 15 seconds. The tube is the centrifuged for 15 minutes at 11000 rpm. 0.4 ml of the supernatant is transferred to a 2 ml tube and 1 ml of 96% ethanol is added, mixing is achieved by slow rotation of the tube. The tube is then centrifuged for 10 minutes at 11000 rpm. Remove the supernatant by pouring away the liquid, wash the pellet with 70% ethanol (0.2 ml) and centrifuge again for 10 minutes at 11000 rpm. Pour away the ethanol, dry the pellet and resuspend in 0.5 ml of TE-buffer) for 30 minutes at 55° C.

### Amplification Procedures

PCR reactions were run in a volume of 8 μl using TEMPase (GeneChoice) polymerase and reaction buffer I as provided by the supplier (GeneChoice). Usually 5 different markers are included in each multiplex PCR. 1 μl DNA, 0.1 μl TEMPase enzyme, 0.2 mM dNTPs, 1.2 mM MgCl2, 0.3 μM each primer.

The PCR mixtures were subjected to initial denaturation at 94° C. for 15 min (for TEMPase). Subsequently, the samples were cycled for 10 cycles with touchdown, i.e. the temperature is lowered 1° C. at each cycle (denaturation at 94° C. 30″, annealing at 67° C. 45″, elongation 72° C. 30″), after which the samples were cycled for 20 cycles with normal PCR conditions (denaturation at 94° C. 30″, annealing at 58° C. 45″, elongation 72° C. 30) PCR cycling was terminated by 1 cycle at 72° C. 30′ and the PCR machine was programmed to cooling down the samples at 4° C. for ‘ever’.

The nucleotide sequence of the primers used for detecting the markers is shown in Table 9. The sequence is listed from the 5′ end.

TABLE 9

| | | |
|---|---|---|
| | Forward Primer F | |
| Marker name | Reverse Primer R | SEQ ID NO.: |

| BTA1: | | |
|---|---|---|
| BMS4008 | F CGGCCCTAAGTGATATGTTG | SEQ ID NO.: 1 |
| | R GAAGAGTGTGAGGGAAAGACTG | SEQ ID NO.: 2 |
| BM8246 | F AATGACAAATTGAGGGAGACG | SEQ ID NO.: 3 |
| | R AGAGCCCAGTATCAATTCTTCC | SEQ ID NO.: 4 |
| BMS4031 | F TCTTGCTGAACAAAGGTTCC | SEQ ID NO.: 5 |
| | R TCCCAGGTATTTGAAGTGTTTC | SEQ ID NO.: 6 |
| D1K2273 | F TAGGCTTCTTTCCCTCCATC | SEQ ID NO.: 7 |
| | R ATGGGTTTGCAAAGAGTTGG | SEQ ID NO.: 8 |
| D1K4151 | F CATTTTCCCCTCAAATAAGACAA | SEQ ID NO.: 9 |
| | R TCTCTTTGATGGAAAAGAGGAAA | SEQ ID NO.: 10 |
| MCM130 | F AAACTTTGTGCTGTTGGGTGTATC | SEQ ID NO.: 11 |
| | R CTCACCTCTGCCTTTCTATCTCTCT | SEQ ID NO.: 12 |
| D1K4367 | F TGGTTCTTCTGTGATGAGACAG | SEQ ID NO.: 13 |
| | R GCATTGGTCACGTTAAATCA | SEQ ID NO.: 14 |
| TGLA130 | F CCAACTGGCCAGTCATAATAAATG | SEQ ID NO.: 15 |
| | R GGGCCGCAAAGGGTTGGATGCA | SEQ ID NO.: 16 |
| BM51789 | F CTGGAAACTGGAAACTAGTGGG | SEQ ID NO.: 17 |
| | R GTGAGGCATTATCAAGAAGCTG | SEQ ID NO.: 18 |
| CSSM019 | F TTGTCAGCAACTTCTTGTATCTTT | SEQ ID NO.: 19 |
| | R TGTTTTAAGCCACCCAATTATTTG | SEQ ID NO.: 20 |
| BM1824 | F GAGCAAGGTGTTTTTCCAATC | SEQ ID NO.: 21 |
| | R CATTCTCCAACTGCTTCCTTG | SEQ ID NO.: 22 |
| UWCA46 | F CCATTTCTCTGTTGGTAACTGC | SEQ ID NO.: 23 |
| | R CTCTCACAGGTGGGGTC | SEQ ID NO.: 24 |
| BM5918 | F AGTCTTCTCTGACAGCAGTTGG | SEQ ID NO.: 25 |
| | R CCAGGTACCAGAGAGAGGAGA | SEQ ID NO.: 26 |
| BM54043 | F TTACAGAAAGAGTGTGTGTGCG | SEQ ID NO.: 27 |
| | R GGCTACAGTTOACAGGTTGC | SEQ ID NO.: 28 |
| URB014 | F CATTGGTAGGTGGGTTCTTTCC | SEQ ID NO.: 29 |
| | R GCAACCTAAGTGTCCATCAACAG | SEQ ID NO.: 30 |
| BTA5: | | |
| BM51095 | F AGGGATTGGTTTATGCTCTCTC | SEQ ID NO.: 31 |
| | R GTTGCAGAGTCGGACATGAC | SEQ ID NO.: 32 |
| BM6026 | F GCAACTAAGACCCAACCAAC | SEQ ID NO.: 33 |
| | R ACTGATGTGCTCAGGTATGACG | SEQ ID NO.: 34 |
| BMS610 | F TTTCACTGTCATCTCCCTAGCA | SEQ ID NO.: 35 |
| | R ATGTATTCATGCACACCACACA | SEQ ID NO.: 36 |
| BP1 | F AAAATCCCTTCATAACAGTGCC | SEQ ID NO.: 37 |
| | R CATCGTGAATTCCAGGGTTC | SEQ ID NO.: 38 |
| D1K2718 | F AGGAAGGACAAGGACATTGC | SEQ ID NO.: 39 |
| | R AGAGGGTCAAAGGCTTAATGG | SEQ ID NO.: 40 |
| AGLA293 | F GAAACTCAACCCAAGACAACTCAAG | SEQ ID NO.: 41 |
| | R ATGACTTTATTCTCCACCTAGCAGA | SEQ ID NO.: 42 |
| D1K5002 | F TGTGCTGGAGGTGATAGCTG | SEQ ID NO.: 43 |
| | R TGCAGGAATATGAGAGCTGAGA | SEQ ID NO.: 44 |
| D1K4759 | F AGTTGGACCTGCCATTGTTC | SEQ ID NO.: 45 |
| | R ACTTATGTGCGTGCGTGCT | SEQ ID NO.: 46 |
| BMC1009 | F GCACCAGCAGAGAGGACATT | SEQ ID NO.: 47 |
| | R ACCGGCTATTGTCCATCTTG | SEQ ID NO.: 48 |
| RM500 | F CAGACACGACTAAGCGACCA | SEQ ID NO.: 49 |
| | R CCTACAATAAAGCACGGGGA | SEQ ID NO.: 50 |
| ETH10 | F GTTCAGGACTGGCCCTGCTAACA | SEQ ID NO.: 51 |
| | R CCTCCAGCCCACTTTCTCTTCTC | SEQ ID NO.: 52 |
| CSSM022 | F TCTCTCTAATGGAGTTGGTTTTTG | SEQ ID NO.: 53 |
| | R ATATCCCACTGAGGATAAGAATTC | SEQ ID NO.: 54 |
| BM51216 | F GAGTAGAACACAACTGAGGACACA | SEQ ID NO.: 55 |
| | R CAATGCTGTGGGTACTGAGG | SEQ ID NO.: 56 |
| BMS1248 | F GTAATGTAGCCTTTTGTGCCG | SEQ ID NO.: 57 |
| | R TCACCAACATGAGATAGTGTGC | SEQ ID NO.: 58 |
| BM315 | F TGGTTTAGCAGAGAGCACATG | SEQ ID NO.: 59 |
| | R GCTCCTAGCCCTGCACAC | SEQ ID NO.: 60 |
| BTA7: | | |
| BM7160 | F TGGATTTTTAAACACAGAATGTGG | SEQ ID NO.: 61 |
| | R TCAGCTTCTCTTTAAATTTCTCTGG | SEQ ID NO.: 62 |
| BL1067 | F AGCCAGTTTCTTCAAATCAACC | SEQ ID NO.: 63 |
| | R ATGGTTCCGCAGAGAAACAG | SEQ ID NO.: 64 |
| BM5713 | F CCAAGGGAGGAAAAATAAGTTAA | SEQ ID NO.: 65 |
| | R ACCAGCAGTAGGTTGAGGTTAA | SEQ ID NO.: 66 |
| D1K5321 | F AACCTTCACAGGCTCCTTCC | SEQ ID NO.: 67 |
| | R CCCATCTCTTGTGCCAAATC | SEQ ID NO.: 68 |
| D1K4421 | F CATCTGAATGGCCAGAATGA | SEQ ID NO.: 69 |
| | R GTCCCCTGCATGTGTCTCTC | SEQ ID NO.: 70 |
| D1K2207 | F ACATTGGCTTACGCTCACACT | SEQ ID NO.: 71 |
| | R CCTGTCTGGGTTTGTTTGCT | SEQ ID NO.: 72 |
| D1K5412 | F ATGGACAGAACAGCCTGACA | SEQ ID NO.: 73 |
| | R TGGTGAACTCAGCCTCACTG | SEQ ID NO.: 74 |
| D1K2819 | F TTACTTTTCGTGGGCCAGAG | SEQ ID NO.: 75 |
| | R GGAACTGTGCCACATAGCAA | SEQ ID NO.: 76 |
| D1K4606 | F TCTTGGAAAGGGGAAAAAGC | SEQ ID NO.: 77 |
| | R TGCTTCATAGCACTTATCTCTTCA | SEQ ID NO.: 78 |
| BM7247 | F AGTAAGGCCTGCAGTATTTATATCC | SEQ ID NO.: 79 |
| | R AATCTTTCCCTAGAACTTACAAAGG | SEQ ID NO.: 80 |
| UWCA20 | F CTGAAACACTCTAAAAGGGTATGC | SEQ ID NO.: 81 |
| | R ATCCCAACATCCACCCATTCC | SEQ ID NO.: 82 |
| BM6117 | F GTTCTGAGGTTTGTAAAGCCC | SEQ ID NO.: 83 |
| | R GGTGAGCTACAATCCATAGGG | SEQ ID NO.: 84 |
| BM52840 | F AGGAACCCATAGGCAGACAC | SEQ ID NO.: 205 |
| | R GCCTGGCAAAGAGAAAATTC | SEQ ID NO.: 206 |
| BM52258 | F CCAGCAGAAGAGAAAGATACTGA | SEQ ID NO.: 85 |
| | R AGTGGTAGAACTTCCATCTCACA | SEQ ID NO.: 86 |
| OARAEI29 | F AATCCAGTGTGTGAAAGACTAATCCAG | SEQ ID NO.: 87 |
| | R GTAGATCAAGATATAGAATATTTTTCAACACC | SEQ ID NO.: 88 |
| IL5T5006 | F TGTCTGTATTTCTGCTGTGG | SEQ ID NO.: 89 |
| | R ACACGGAAGCGATCTAAACG | SEQ ID NO.: 90 |
| BL1043 | F AGTGCCAAAAGGAAGCGC | SEQ ID NO.: 91 |
| | R GACTTGACCGTTCCACCTG | SEQ ID NO.: 92 |
| BTAI5: | | |
| BM52684 | F CCAAGGTCATTGTTGCAGC | SEQ ID NO.: 93 |
| | R TGGGGATTTGCTTCTCAGTC | SEQ ID NO.: 94 |
| INRA145 | F TAATAAAACTGGTCCCTCTGGC | SEQ ID NO.: 95 |
| | R TGCTGGCTCTCCAGTATGC | SEQ ID NO.: 96 |
| IDVGA-10 | F TCTCCTGGCTACAGGGCTAA | SEQ ID NO.: 97 |
| | R CCCACTGGCCTAGAACCC | SEQ ID NO.: 98 |
| ILST5027 | F GGTGTGTTGGTTAAGACTGG | SEQ ID NO.: 99 |
| | R GAATCATAGACCTGACTTCC | SEQ ID NO.: 100 |
| BM5812 | F TGGACAGGACTGAGTATGCA | SEQ ID NO.: 101 |
| | R AGGTATCCAACTAACACAGCCA | SEQ ID NO.: 102 |
| BMS2076 | F AGCACCTGTACCATCTGTTCC | SEQ ID NO.: 103 |
| | R TCCATAGGCTCACAAAGAGTTG | SEQ ID NO.: 104 |
| BL1095 | F TCCCTCTACCATATATTTCCCC | SEQ ID NO.: 105 |
| | R CATTAGCATGGAAAAACCTCTG | SEQ ID NO.: 106 |
| BM5820 | F CCACTACTTGCCTCAGGGAG | SEQ ID NO.: 107 |
| | R ACAGGACTCTCAAGCATCAGC | SEQ ID NO.: 108 |
| BMS927 | F GATGATCCACCATAACTACCAGA | SEQ ID NO.: 109 |
| | R TGGCTCTCAAAGGTCATTGT | SEQ ID NO.: 110 |
| BM5429 | F TACATTAACCCCAAAATTAAATGC | SEQ ID NO.: 111 |
| | R CCCTTGATTTCTCTCATGAGTATT | SEQ ID NO.: 112 |
| BTA21: | | |
| BMS1117 | F TGTGTGCTCTCTCACACATGC | SEQ ID NO.: 113 |
| | R AACCAAAGCAGGGATCAGG | SEQ ID NO.: 114 |
| AGLA233 | F TGCAAACATCCACGTAGCATAAATA | SEQ ID NO.: 115 |
| | R GCATGAACAGCCAATAGTGTCATC | SEQ ID NO.: 116 |
| 1L5T5095 | F GAAAGATGTTGCTAGTGGGG | SEQ ID NO.: 117 |
| | R ATTCTCCTGTGAACCTCTCC | SEQ ID NO.: 118 |
| BMIO3 | F CTAGCTGCTGGCTACTTGGG | SEQ ID NO.: 119 |
| | R GGCTGCTCTGGGCTATTG | SEQ ID NO.: 120 |
| IDVGA-45 | F GTGGTGGCAAAGAGTCAGA | SEQ ID NO.: 121 |
| | R AACAGCCCTGATTTCCATA | SEQ ID NO.: 122 |
| INRAIO3 | F TTGTCCAGCCCAGCATTTAGC | SEQ ID NO.: 123 |
| | R GGAGAAGACTTATGGGAGC | SEQ ID NO.: 124 |
| BM52815 | F TGATATTCAAACTCAATGAACCC | SEQ ID NO.: 125 |
| | R CTTGCATATGCTCATCATTATCA | SEQ ID NO.: 126 |
| BM846 | F GACCACTGGACCACCAGG | SEQ ID NO.: 127 |
| | R CTGGTAAAAAGCAATGATGCC | SEQ ID NO.: 128 |
| BTA 27: | | |
| BMS1001 | F GAGCCAATTCCTACAATTCTCTT | SEQ ID NO.: 129 |
| | R AGACATGGCTGAAATGACTGA | SEQ ID NO.: 130 |
| BM52650 | F CCTCTGTGTCCACACTGCC | SEQ ID NO.: 131 |
| | R CCTAGTGACATCCTGGGGTG | SEQ ID NO.: 132 |
| INRA06 | F AGGOAGACOTTACCATAGGAGA | SEQ ID NO.: 133 |
| | R GTCGCAATGAGTTGGACACAAC | SEQ ID NO.: 134 |
| BM52137 | F CCAGAGAAGCAGAACCAGTAGG | SEQ ID NO.: 135 |
| | R CTTGTCAGCGTCCATAATTCC | SEQ ID NO.: 136 |
| C55M043 | F AAAACTCTGGGAACTTGAAAACTA | SEQ ID NO.: 137 |
| | R GTTACAAATTTAAGAGACAGAGTT | SEQ ID NO.: 138 |
| 10BT313 | F GAATCAATAAAGAAGATGCAGCACG | SEQ ID NO.: 149 |
| | R GCCCTCTAGGTCTATCTGTGTTTGC | SEQ ID NO.: 150 |
| INRAI34 | F CCAGGTGGGAATAATGTCTCC | SEQ ID NO.: 139 |
| | R TTGGGAGCCTGTGGTTTATC | SEQ ID NO.: 140 |
| BM1857 | F GCTGTGGCTGTGCTTGTG | SEQ ID NO.: 141 |
| | R AGTAACTGCCCCCGGAAG | SEQ ID NO.: 142 |
| BMS2116 | F TCCCTGTGTTGAGGAGCTG | SEQ ID NO.: 143 |
| | R TTAATCMTGCACACGCATG | SEQ ID NO.: 144 |
| HUJI-13 | F TCCTTGTATTCACACGTGGG | SEQ ID NO.: 145 |
| | R TTCTCAGCCAAAGTCAAGGG | SEQ ID NO.: 146 |
| MSBQ | F TTAAGGTTGTTGCATACTCCTG | SEQ ID NO.: 151 |
| | R AAGTTCTCAGCCAAAGTCAAGG | SEQ ID NO.: 152 |
| BM203 | F GGGTGTGACATTTTGTTCCC | SEQ ID NO.:147 |
| | R CTGCTCGCCACTAGTCCTTC | SEQ ID NO.:148 |
| BTA6: | | |
| OARJMP36 | F: CCCACTTTCTGGAAGGCAGAAATG | SEQ ID NO.: 153 |
| | R: CTTATTGTGTTTTCTGCCAGGGAG | SEQ ID NO.: 154 |
| BM415 | F: GCTACAGCCCTTCTGGTTTG | SEQ ID NO.: 155 |
| | R: GAGCTAATCACCAACAGCAAG | SEQ ID NO.: 156 |
| BM4311 | F: TCCACTTCTTCCCTCATCTCC | SEQ ID NO.: 157 |
| | R: GAAGTATATGTGTGCCTGGCC | SEQ ID NO.: 158 |
| BM2320 | F: GGTTCCCAGCAGCAGTAGAG | SEQ ID NO.: 159 |
| | R: CCCATGTCTCCCGTTACTTC | SEQ ID NO.: 160 |
| BL1038 | F: GGCAAGCTAGAGTCAGACACG | SEQ ID NO.: 161 |
| | R: GCAAAAGTCTAGGTGAAATGCC | SEQ ID NO.: 162 |
| BTA9: | | |
| BMS2151 | F: CCATTAAGAGGAAATTGTGTTCA | SEQ ID NO.: 163 |
| | R: ATGGAGTCACTGAAAGGTACTGA | SEQ ID NO.: 164 |
| | F: GATCACCTTGCCACTATTTCCT | SEQ ID NO.: 165 |
| ETH225 | | |
| | R: ACATGACAGCOAGCTGCTACT | SEQ ID NO.: 166 |
| | F: TAGGCTATGTACTGACCATGC | SEQ ID NO.: 167 |
| IL5T5037 | | |
| | R: CTGAACTGAGATGACTTTGGC | SEQ ID NO.: 168 |
| BM2504 | F: CAGCTTTCCATCCCCTTTC | SEQ ID NO.: 169 |
| | R: CTCCCATCCCAAACACAGAC | SEQ ID NO.: 170 |
| BMS1267 | F: TTCTGAATTTGATTCCCAACA | SEQ ID NO.: 171 |
| | R: ACTGTTTCCTTAAAAGCTTCCC | SEQ ID NO.: 172 |
| UWCA9F: | F: CCTTCTCTGAATTTTTGTTGAAAGC | SEQ ID NO.: 173 |
| | R: GGACAGAAGTGAGTGACTGAGA | SEQ ID NO.: 174 |
| BM51290 | F: TTGGCACTTACTACCTCATATGTT | SEQ ID NO.: 175 |
| | R: TTTTCTGGATGTTGAGCCTATT | SEQ ID NO.: 176 |
| BM6436 | F: AAAGACTGCTTGCCTGAAGC | SEQ ID NO.: 177 |
| | R: CAACCAGTGATGCTGTACTCTG | SEQ ID NO.: 178 |
| BM52753 | F: TCAAAAAGTTGGACATGACTGA | SEQ ID NO.: 179 |
| | R: AGGTTTTCAAATGAGAGACTTTTC | SEQ ID NO.: 180 |
| BM52819 | F: GCTCACAGGTTCTGAGGACTC | SEQ ID NO.: 181 |
| | R: AACTTGAAGAAGGAATGCTGAG | SEQ ID NO.: 182 |
| BTA11: | | |
| BM52047 | F: ACTATGGACATTTGGGGCAG | SEQ ID NO.: 183 |
| | R: AGTAGGTGGAGATCAAGGATGC | SEQ ID NO.: 184 |
| HUJV174 | F: CAGACCAGTTTCTCAGACAAGC | SEQ ID NO.: 185 |
| | R: TCATTCCTGTGTCAATACAGCC | SEQ ID NO.: 186 |
| TGLA436 | F: TGTATGGCTGAATGATATTCCATTT | SEQ ID NO.: 187 |
| | R: CTACTGACAGATGATTAGATAAAGA | SEQ ID NO.: 188 |
| HEL13 | F: TAAGGACTTGAGATAAGGAG | SEQ ID NO.: 189 |
| | R: CCATCTACCTCCATCTTAAC | SEQ ID NO.: 190 |
| BTA26: | | |
| BM5332 | F: GACAAAACCCTTTTAGCACAGG | SEQ ID NO.: 191 |
| | R: AATTGCATGGAAAGTTCTCAGC | SEQ ID NO.: 192 |
| RM026 | F: TTGTACATTTCTGTCAATGCCTT | SEQ ID NO.: 193 |
| | R: ACAATGTCATTGGTCAATTCATT | SEQ ID NO.: 194 |
| IDVGA-59 | F: AACCCAAATATCCATCAATAG | SEQ ID NO.: 195 |
| | R: CAGTCCCTCAACCCTCTTTTC | SEQ ID NO.: 196 |
| BM5882 | F: TAGTGTCCACCAGAGACCCC | SEQ ID NO.: 197 |
| | R: CCAAAGACACAGTTTAAAGGGC | SEQ ID NO.: 198 |
| BM804 | F: CCAGCATCAACTGTCAGAGC | SEQ ID NO.: 199 |
| | R: GGCAGATTCTTTGCCTTCTG | SEQ ID NO.: 200 |
| BM9284 | F: AGGTGCTGGAATGGCAAC | SEQ ID NO.: 201 |
| | R: TGTGATTTTGGTCTTCCTTGC | SEQ ID NO.: 202 |
| BM7237 | F: TTTCTGCTAATGGCATCATTT | SEQ ID NO.: 203 |
| | R: TGGATAAAGAAGATGTGGTGTG | SEQ ID NO.: 204 |
| Note: | | |
| two different marker names amplifying the | | |
| same locus | | |

0.5 μl PCR-product is added to 9.5 μl formamide and analysed on an ABI-3730XL sequencing Instrument (Applied Biosystems Inc.).

### Markers and Map

Markers were chosen from previous published maps (Barendse et al. 1997) and from the website of the Meat Animal Research Center (http://sol.marc.usda.gov/). All autosomes [Bos taurus chromosomes (BTA) 1-29] were covered in a whole genome scan. The genome was screened using 327 micro-satellite markers with an average marker spacing of 7.97 cM. Marker genotypes were determined on an automated sequence analyser (ABI, Perkin Elmer). The map was created using Cri-MAP version 2.4 (Green et al., 1990) and the Haldane map function. The calculated map distances were used in the QTL analysis. Tables 10-15 show the markers used per chromosome.

The following tables show markers used for the relevant QTL. Any additional information on the markers can be found on ‘http://www.marc.usda.gov/’.

TABLE 10

| | Position employed in | Relative position (cM) |
|---|---|---|
| Marker on BTA1 | analysis (cM) | http://www.marc.usda.gov/ |
| BMS4008 | 71.7 | 80.379 |
| BM8246 | 76.2 | 83.834 |
| BMS4031 | 77.7 | 87.124 |
| DIK2273 | 84.5 | 84.471 |
| DIK4151 | 90.0 | 89.989 |
| MCM130 | 92.6 | 92.649 |
| DIK4367 | 97.2 | 97.246 |
| TGLA130 | 98.2 | 110.816 |
| BMS1789 | 100.9 | 113.501 |
| CSSM019 | 108.3 | 122.094 |
| BM1824 | 108.6 | 122.391 |
| UWCA46 | 113.2 | 127.441 |
| BMS918 | 118.1 | 132.471 |
| BMS4043 | 128.7 | 142.244 |
| URB014 | 142.1 | 154.672 |

TABLE 11

| | Position employed in | Relative position (cM) |
|---|---|---|
| Marker on BTA5 | analysis (cM) | http://www.marc.usda.gov/ |
| BMS1095 | 0.0 | 0 |
| BM6026 | 6.7 | 6.05 |
| BMS610 | 12.8 | 12.018 |
| BP1 | 18.8 | 17.287 |
| DIK2718 | 30.1 | 30.143 |
| AGLA293 | 32.0 | 32.253 |
| DIK5002 | 33.7 | 33.655 |
| DIK4759 | 40.3 | 40.293 |
| BMC1009 | 40.6 | 41.693 |
| RM500 | 55.6 | 56.303 |
| ETH10 | 70.0 | 71.764 |
| CSSM022 | 72.4 | 74.2 |
| BMS1216 | 75.6 | 78.205 |
| BMS1248 | 88.4 | 90.849 |
| BM315 | 100.1 | 103.169 |

TABLE 11b

| | Position employed in | Relative position (cM) |
|---|---|---|
| Marker on BTA6 | analysis (cM) | http://www.marc.usda.gov/ |
| ILSTS093 | 0 | 0 |
| INRA133 | 8.2 | 8.053 |
| BM1329 | 35.5 | 35.398 |
| OARJMP36 | 52.4 | 56.12 |
| BM415 | 76.3 | 81.961 |
| BM4311 | 89.1 | 97.728 |
| BM2320 | 120.7 | 127.264 |
| BL1038 | 122.3 | 129.985 |

TABLE 11c

| | Position employed in | Relative position (cM) |
|---|---|---|
| Marker on BTA9 | analysis (cM) | http://www.marc.usda.gov/ |
| BMS2151 | 0 | 4.892 |
| ETH225 | 8.1 | 12.754 |
| ILSTS037 | 21 | 26.266 |
| BM2504 | 25.2 | 30.92 |
| BMS1267 | 33.8 | 38.742 |
| UWCA9 | 44.9 | 49.996 |
| BMS1290 | 59.0 | 64.935 |
| BM6436 | 71.1 | 77.554 |
| BMS2753 | 73.1 | 79.249 |
| BMS2819 | 84.4 | 90.98 |
| BM4208 | 84.6 | 90.69 |
| BMS2295 | 91.5 | 98.646 |
| BMS1967 | 102.5 | 109.287 |

TABLE 12

| | Position employed in | Relative position (cM) |
|---|---|---|
| Markers on BTA7 | analysis (cM) | http://www.marc.usda.gov/ |
| BM7160 | 0.0 | 0 |
| BL1067 | 14.2 | 14.683 |
| BMS713 | 15.2 | 16.756 |
| DIK5321 | 22.3 | 22.286 |
| DIK4421 | 22.7 | 22.692 |
| DIK2207 | 26.7 | 26.74 |
| DIK5412 | 30.2 | 30.166 |
| DIK2819 | 47.9 | 47.908 |
| DIK4606 | 55.3 | 55.292 |
| BM7247 | 58.0 | 57.263 |
| UWCA20 | 59.9 | 58.552 |
| BM6117 | 61.0 | 62.246 |
| BMS2840 | 64.3 | 65.305 |
| BMS2258 | 75.0 | 77.194 |
| OARAE129 | 96.6 | 95.93 |
| ILSTS006 | 116.0 | 116.629 |
| BL1043 | 134.1 | 135.564 |

TABLE 12b

| | Position employed in | Relative position (cM) |
|---|---|---|
| Marker on BTA11 | analysis (cM) | http://www.marc.usda.gov/ |
| BM716 | 9.5 | 19.44 |
| BMS2569 | 11.7 | 21.082 |
| BM2818 | 20.5 | 30.009 |
| INRA177 2 | 25.7 | 34.802 |
| RM096 | 31.3 | 40.481 |
| INRA131 | 38.0 | 47.289 |
| BM7169 | 41.0 | 50.312 |
| BM6445 | 56.9 | 61.57 |
| BMS1822 | 61.2 | 65.879 |
| TGLA58 | 67.5 | 83.136 |
| BMS2047 | 73.8 | 78.457 |
| HUJV174 | 85.4 | 92.179 |
| TGLA436 | 98.5 | 105.214 |
| HEL13 | 114.5 | 122.37 |

TABLE 13

| | Position employed in | Relative position (cM) |
|---|---|---|
| Marker on BTA15 | analysis (cM) | http://www.marc.usda.gov/ |
| BMS2684 | 34.9 | 48.216 |
| INRA145 | 51.6 | 67.759 |
| IDVGA-10 | 51.7 | 67.759 |
| ILSTS027 | 66.3 | 83.417 |
| BMS812 | 68.8 | 84.894 |
| BMS2076 | 75.4 | 91.848 |
| BL1095 | 77.8 | 94.775 |
| BMS820 | 81.6 | 98.184 |
| BMS927 | 88.3 | 104.998 |
| BMS429 | 93.4 | 109.753 |

TABLE 14

| | Position employed in | Relative position (cM) |
|---|---|---|
| Markers on BTA21 | analysis (cM) | http://www.marc.usda.gov/ |
| BMS1117 | 9.9 | 10.969 |
| AGLA233 | 20.4 | 21.202 |
| ILSTS095 | 24.4 | 23.735 |
| BM103 | 30.5 | 29.77 |
| IDVGA-45 | 31.8 | 30.887 |
| INRA103 | 37.7 | 35.898 |
| BMS2815 | 46.1 | 41.714 |
| BM846 | 61.247 | 61.247 |

TABLE 14b

| | Position employed in | Relative position (cM) |
|---|---|---|
| Marker on BTA26 | analysis (cM) | http://www.marc.usda.gov/ |
| BMS651 | 2.5 | 2.839 |
| HEL11 | 20.7 | 22.862 |
| BMS332 | 27.0 | 31.65 |
| RM026 | 37.3 | 37.635 |
| IDVGA-59 | 50.6 | 53.094 |
| BMS882 | 51.0 | 53.477 |
| BM804 | 59.6 | 60.476 |
| BM9284 | 59.7 | 41.648 |
| BM7237 | 64.3 | 66.763 |

TABLE 15

| | Position employed in | Relative position (cM) |
|---|---|---|
| Markers on BTA27 | analysis (cM) | http://www.marc.usda.gov/ |
| BMS1001 | 0.054 | 5.389 |
| BMS 2650 | 0.123 | 12.285 |
| INRA016 | 0.172 | 17.186 |
| BMS2137 | 0.208 | 20.781 |
| CSSM043 | 0.345 | 34.525 |
| IOBT313 | 0.345 | 34.525 |
| INRA134 | 0.453 | 45.253 |
| BM1857 | 0.523 | 52.326 |
| BMS2116 | 0.544 | 54.389 |
| HUJI-13 | 0.557 | 55.75 |
| BM203 | 0.641 | 64.098 |

### Phenotypic Data

Daughters of bulls were scored for mas1, mas2, mas3, mas4, SCC, and the index udder health. Estimated breeding values (EBV) for traits of sons were calculated using a single trait Best Linear Unbiased Prediction (BLUP) animal model ignoring family structure (Table 16). These EBVs were used in the QTL analysis. The daughter registrations used in the individual traits were:

Mas1: Treated cases of clinical mastitis in the period −5 to 50 days after 1^{st} calving.

Mas2: Treated cases of clinical mastitis in the period −5 to 305 days after 1^{st} calving.

Mas3: Treated cases of clinical mastitis in the period −5 to 305 days after 2^{nd} calving.

Mas4: Treated cases of clinical mastitis in the period −5 to 305 days after 3^{rd} or later calving.

SCS: Mean SCS in period 5-180 days after 1^{st} calving.

Udder health index: An index weighing together information from Mas1-Mas4, SCC, fore udder attachment, udder depth, and udder band.

TABLE 16
Estimated breeding values (EBV) for traits of sons were calculated using a single trait
Best Linear Unbiased Prediction (BLUP) animal model ignoring family structure.

| Herdbook | Name of | | | | | |
|---|---|---|---|---|---|---|
| number | bull | SCS | Mas1 | Mas2 | Mas3 | Mas4 |
| 17001 | Bell | −0.013680238 | −0.429694571 | 0.537592985 | 0.262327691 | 7.008117768 |
| 221402 | Chief Mark | −0.114948368 | 1.144984731 | −0.987864853 | 3.169259889 | 4.959184463 |
| 223803 | B Cleitus R | 0.125688409 | −0.009775993 | 1.328407329 | 6.438078071 | 3.928507544 |
| 225602 | Vanguard | 0.054190513 | −2.281007402 | −3.362463417 | 3.674808889 | 4.187879609 |
| 226201 | T Blackstar | −0.026106869 | −0.301245549 | 0.748573402 | 10.14985473 | 2.684794076 |
| 226804 | Southwind | 0.047245505 | 1.45510651 | 0.21328716 | 3.678426096 | 5.916101326 |
| 227402 | M Aerostar | −0.031867769 | 5.723790796 | 9.45312554 | 6.190146343 | 3.804737067 |
| 227405 | R Leadman | 0.020957899 | −1.308117837 | −0.125198875 | 1.757522665 | 2.361419456 |
| 228860 | Tesk Holm | 0.050229207 | 4.797201292 | 9.516047957 | 10.09577652 | 6.71104168 |
| 229400 | S-B Mascot | 0.009910227 | 4.815448009 | 5.028808372 | 7.066419623 | 4.040847809 |
| 229612 | Belt | −0.037254252 | 3.024593731 | 4.432084923 | 5.40099934 | 3.543367498 |
| 230104 | T Burma | −0.047398423 | 3.155805504 | 0.755202584 | −1.127451405 | −0.098113856 |
| 230150 | R Prelude | −0.070599072 | 0.592997381 | 2.454143335 | −0.050784044 | −0.406766344 |
| 231555 | J Jed | 0.049128097 | 1.194645415 | 4.240790565 | 5.54827409 | 9.549000015 |
| 231900 | B Mountain | 0.027741222 | −2.713489262 | 1.364271511 | 3.734456698 | 2.8509699 |
| 232606 | N Luke | 0.074085566 | 0.142524628 | 1.407064244 | 6.566201895 | 2.501502826 |
| 232851 | Funkis | −0.160865306 | −6.085145685 | −9.820959183 | −9.807432842 | −9.71176622 |
| 233348 | G Slocum | −0.020787003 | −0.491369762 | 2.524305655 | 3.436555642 | 4.224025095 |
| 233463 | E Celsius | 0.126706517 | 5.451958777 | 10.78821462 | 7.536178772 | 8.367135538 |
| 233932 | Dombinator | −0.097336995 | −1.546610474 | −2.878646567 | 1.840753841 | −1.422337242 |
| 234347 | Ked Juror | 0.01321437 | −2.203635759 | −1.275471378 | −0.728432585 | −1.760241345 |
| 234582 | M Bellwood | −0.082941508 | 4.305206658 | 2.355899553 | 0.797580292 | −1.22424015 |
| 234984 | Esquimau | 0.161337281 | −1.870547567 | −0.695053467 | 5.535659522 | 8.393015363 |
| 235922 | East Cash | 0.133477207 | 0.127343059 | 2.487764232 | 5.518102877 | 5.534846523 |
| 236598 | Fatal | 0.19866763 | 2.727462349 | 2.904162654 | 0.200944292 | 2.291056469 |
| 236735 | Evreux Cle | 0.076479923 | 3.182792522 | 5.65707962 | 1.375810952 | 2.213590542 |
| 236947 | Esentation | −0.088055054 | −0.401045562 | 0.292075443 | 0.279423353 | 0.534813295 |
| 237017 | Lord Lily | −0.170419317 | −2.589933641 | −4.324451445 | −0.150162503 | −1.15483455 |
| 237985 | Luxemburg | −0.011601569 | −3.065840995 | −5.786588685 | −4.470245232 | −5.688578481 |
| 238986 | Mattie G. Hondo | 0.100387699 | 1.441219961 | 3.00763287 | 7.644601899 | 5.795565228 |
| 239278 | Aero | −0.054563127 | 4.195260435 | 2.612311231 | 0.69831259 | 5.974003921 |
| 239280 | Lukas | 0.008977319 | 0.446188602 | −0.9678392 | 0.92466249 | −0.848259276 |
| 239657 | Basar | −0.184694197 | 0.335768607 | −2.616821234 | −4.252202253 | −2.780435079 |
| 240131 | Boudewin | 0.105191872 | 3.673262833 | 5.72254585 | 8.362535847 | 7.665138364 |

### QTL Analysis

The data was analysed with a series of models. Initially, a single trait model using a multipoint regression approach for all traits were analysed over all chromosomes. Chromosomes with significant effects within families were analysed with the variance component method to validate QTL found across families and for characterization of QTL. When a chromosome was found to affect more than one trait multiple trait variance components models were used.

### Regression Analysis

Population allele frequencies at the markers were estimated using an EM-algorithm. Allele frequencies were subsequently assumed known without error. Phase in the sires was determined based on offspring marker types. Subsequently this phase was assumed known without error. Segregation probabilities at each map position were calculated using information from all markers on the chromosome simultaneously using Haldane's mapping function (Haldane, 1919). Phenotypes were regressed onto the segregation probabilities. Significance thresholds were calculated using permutation tests (Churchil and Doerge, 1994).

Variance component analysis. Single trait single QTL analysis.

Each trait was analysed separately using linkage analysis. The full model can be expressed as:

y=Xβ+Zu+Wq+e, (1)

where y is a vector of n EBVs, X is a known design matrix, β is a vector of unknown fixed effects, which is in this case only the mean, Z is a matrix relating to individuals, u is a vector of additive polygenic effects, W is a known matrix relating each individual record to its unknown additive QTL effect, q is a vector of unknown additive QTL effects of individuals and e is a vector of residuals. The random variables u, q and e are assumed to be multivariate normally distributed and mutually independent (Lund et al., 2003).

### Multi Trait Single QTL Analysis

For chromosomes affecting two or more traits a multi-trait analysis was performed. Model (1) can be extended to a multi-trait single QTL model where y is an n*t vector of n observations on t traits (Sørensen et al., 2003).

IBD matrix

First the gametic relationship matrix (Fernando and Grossman, 1989) was calculated and then using the linear relationship between the gametic relationship matrix and the IBD matrix, the IBD matrix was designed (George et al., 2000). The covariance structure among the random QTL allelic effect of all animals in the pedigree, are described by the gametic relationship matrix. The information of the transmission of linked markers is used to calculate the IBD probabilities at the position of a putative QTL position (Sørensen et al., 2003).

### Significance Level

Significance thresholds for the variance-component analyses were calculated using a quick method to compute approximate threshold levels that control the genome-wise type I error (Piepho, 2001). A significance level of 5% chromosome wise was considered to be significant.

### Example 1

BTA1

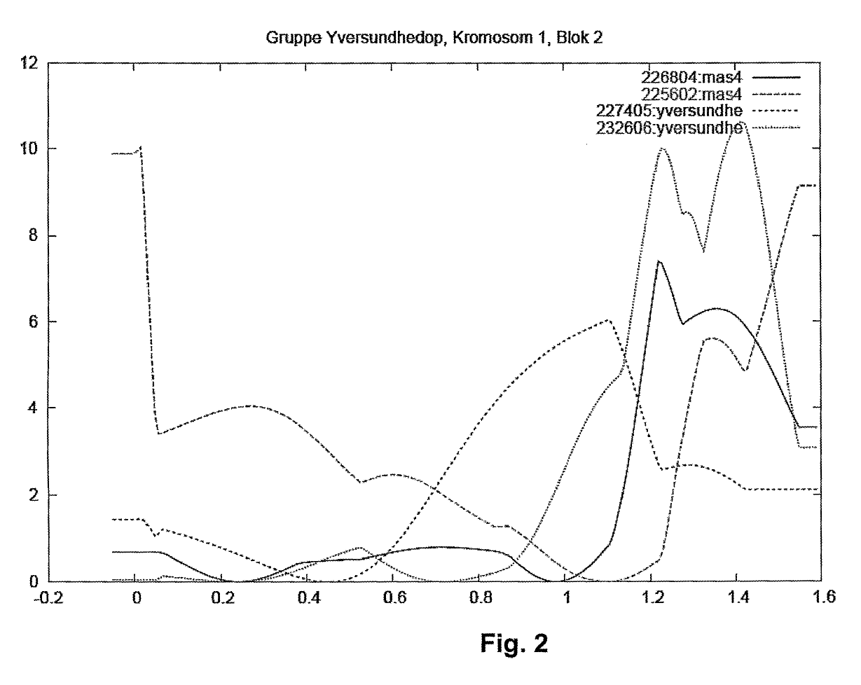

In table 17 the results from the regression analysis for BTA1 are presented. FIG. 1 and FIG. 2 present the QTL graphs for the regression analysis. The variance component method was used to detect QTL across families (including all the sire families in one analysis) for CELL, MAS1, MAS2, MAS3, MAS4, and udder health index in a single trait analysis. There was no significant QTL detected for CELL, MAS1, MAS2, MAS3, MAS4, and udder health index in the across family analysis. From the multi-trait analysis there is no sign for pleiotrophic QTL affecting the traits CELL, MAS1, MAS2, MAS3, MAS4, and udder health index. Results of the within family analysis is shown in table 17

TABLE 17
Significant QTL from the within family analysis using the regression analysis on BTA1

| | | Position | Herdbook | Name | Sub | p- | | |
|---|---|---|---|---|---|---|---|---|
| Chr | Main trait | (Morgan) | number | Sire | trait | value* | F-value | Effect |
| 1 | Udder health | 1.342 | 232606 | N Luke | Cell | 0.997 | 13.55 | 0.035 |
| 1 | Udder health | 0.843 | 238986 | Mattie G. | Mas1 | 1 | 16.06 | −0.85 |
| 1 | Udder health | 1.085 | 232606 | N Luke | Mas1 | 0.989 | 12.27 | 0.68 |
| 1 | Udder health | 0.873 | 221402 | Chief Mark | Mas2 | 0.937 | 7.72 | 1.2 |
| 1 | Udder health | 1.085 | 232606 | N Luke | Mas2 | 0.978 | 9.48 | 0.84 |
| 1 | Udder health | 1.342 | 230104 | T Burma | Mas3 | 0.956 | 7.14 | −1.1 |
| 1 | Udder health | 0.798 | 223803 | B Cleitus | Mas4 | 0.98 | 9.23 | −1.4 |
| 1 | Udder health | 1.062 | 229612 | Belt | Mas4 | 0.947 | 18.14 | −1.1 |
| 1 | Udder health | 1.085 | 226804 | Southwind | Mas4 | 0.98 | 9.2 | −0.98 |
| 1 | Udder health | 1.426 | 225602 | R Vanguard | Mas4 | 0.969 | 9.95 | 1.4 |
| 1 | Udder health | 0.979 | 227405 | R Leadman | UHI | 0.949 | 6.57 | 1.3 |
| 1 | Udder health | 1.093 | 232606 | N Luke | UHI | 0.984 | 10.53 | −1.3 |

*(1 − [p-value]) = chromosome wide significance level
UHI = Udder health index

### Example 2

### BTA5

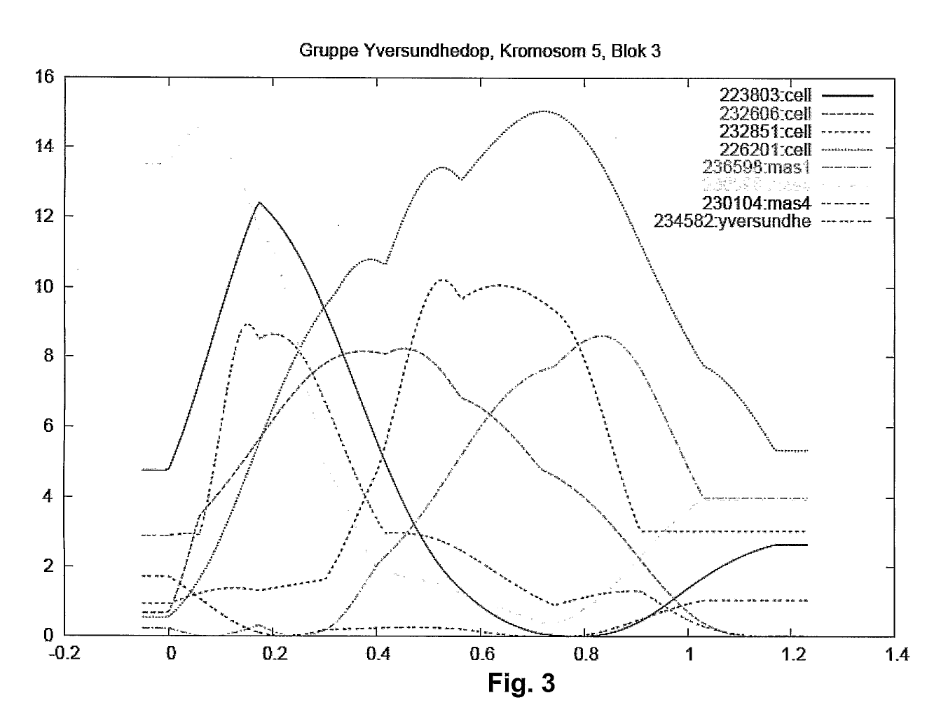

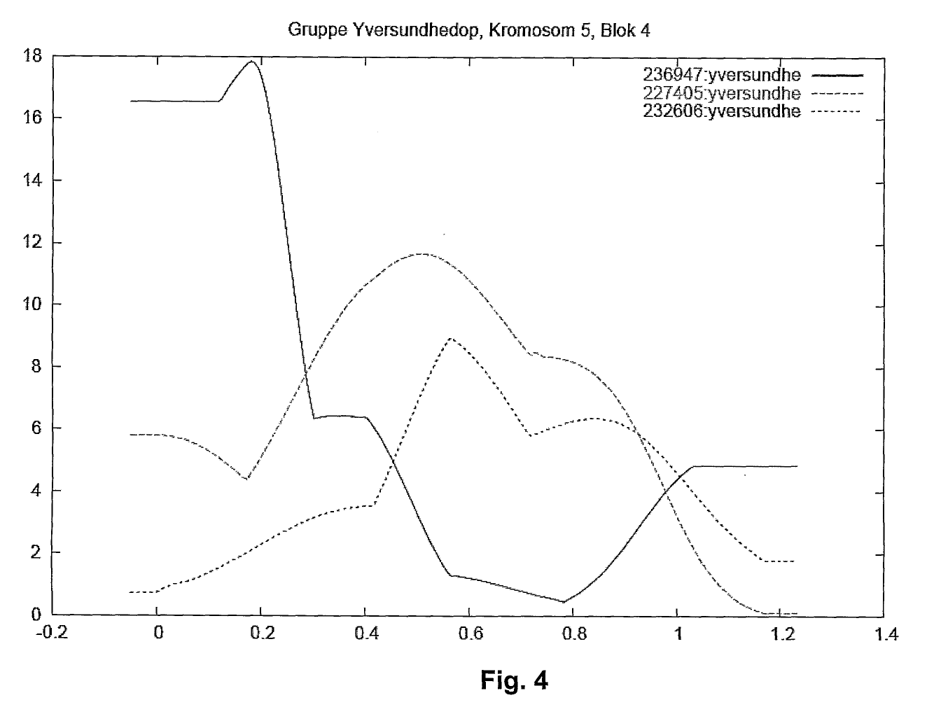

In table 18 the results from the regression analysis for BTA5 are presented. FIG. 3 and FIG. 4 present the QTL graphs for the regression analysis. The variance component method was used to detect QTL across families (including all the sire families in one analysis) for CELL, MAS1, MAS2, MAS3, MAS4, and udder health index in a single trait analysis. A significant QTL was detected in the across family analysis for CELL (Likelihood Ratio=11.02, at position 0.44 Morgan. Three sire families contribute to this QTL: 223803, 226201, and 232606. There was no significant QTL detected for MAS1, MAS2, MAS3, MAS4, and udder health index in the across family analysis. From the multi-trait analysis there is no sign for pleiotrophic QTL affecting the traits CELL, MAS1, MAS2, MAS3, MAS4, and udder health index.

TABLE 18
Significant QTL from the within family analysis using the regression analysis on BTA5

| | | Position | Herdbook | | Sub | p- | | |
|---|---|---|---|---|---|---|---|---|
| Chr | Main trait | (Morgan) | number | Name Sire | trait | value* | F-value | effect |
| 5 | Udder health | 0.19 | 223803 | B Cleitus | Cell | 0.991 | 11.51 | 0.041 |
| 5 | Udder health | 0.442 | 232606 | N Luke | Cell | 0.962 | 8.33 | −0.028 |
| 5 | Udder health | 0.643 | 232851 | Funkis | Cell | 0.967 | 9.27 | −0.063 |
| 5 | Udder health | 0.714 | 226201 | T Blackstar | Cell | 0.998 | 13.67 | 0.044 |
| 5 | Udder health | 0.812 | 236598 | Fatal | Mas1 | 0.967 | 8.89 | 1 |
| 5 | Udder health | 0.183 | 236598 | Fatal | Mas4 | 0.985 | 11.29 | 0.9 |
| 5 | Udder health | 0.948 | 230104 | T Burma | Mas4 | 0.975 | 8.97 | 0.9 |
| 5 | Udder health | 0.157 | 234582 | M Bellwood | UHI | 0.958 | 8.37 | −2.2 |
| 5 | Udder health | 0.216 | 236947 | Esentation | UHI | 0.993 | 14.99 | −3.2 |
| 5 | Udder health | 0.488 | 227405 | R Leadman | UHI | 0.995 | 12.17 | −1.9 |
| 5 | Udder health | 0.559 | 232606 | N Luke | UHI | 0.985 | 10.05 | 1.4 |

*(1 − [p-value]) = chromosome wide significance level
UHI = Udder health index

### Example 3

### BTA7

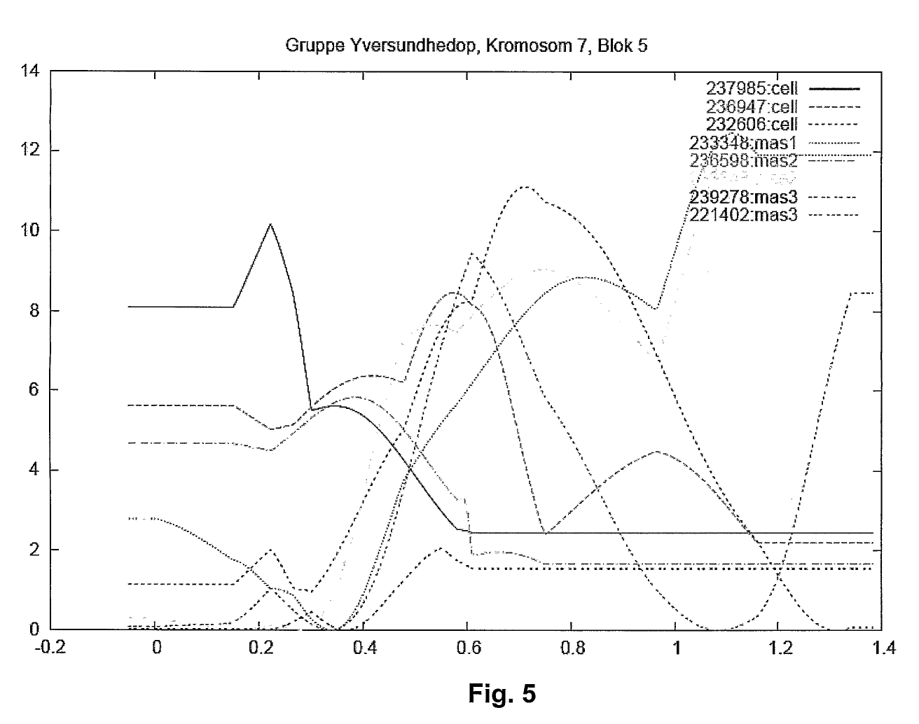

In table 19 the results from the regression analysis for BTA7 are presented. FIG. 5 and

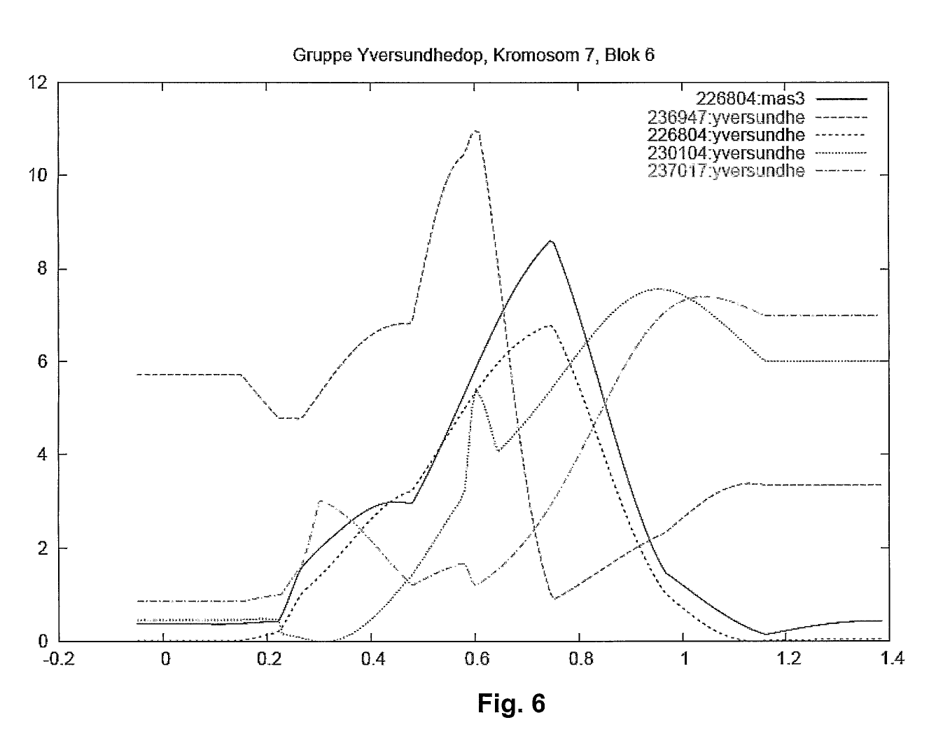

FIG. 6 present the QTL graphs for the regression analysis. The variance component method was used to detect QTL across families (including all the sire families in one analysis) for CELL, MAS1, MAS2, MAS3, MAS4, and udder health index in a single trait analysis. A significant QTL was detected in the across family analysis for udder health index (Likelihood Ratio=18.9, at position 0.75 Morgan). Four sire families contribute to this QTL: 236947, 226804, 230104, and 237017. There was no significant QTL detected for CELL, MAS1, MAS2, MAS3, and MAS4 in the across family analysis. From the multi-trait analysis there is no sign for pleiotrophic QTL affecting the traits CELL, MAS1, MAS2, MAS3, MAS4, and udder health index.

TABLE 19
Significant QTL from the within family analysis using the regression analysis on BTA7

| | | Position | Herdbook | | Sub | p- | | |
|---|---|---|---|---|---|---|---|---|
| Chr | Main trait | (Morgan) | number | Name Sire | trait | value* | F-value | effect |
| 7 | Udder health | 0.222 | 237985 | Luxemburg | Cell | 0.992 | 10.42 | −0.058 |
| 7 | Udder health | 0.574 | 236947 | Esentation | Cell | 0.978 | 8.85 | 0.064 |
| 7 | Udder health | 0.717 | 232606 | N Luke | Cell | 0.993 | 11.24 | 0.033 |
| 7 | Udder health | 1.119 | 233348 | G Slocum | Mas1 | 0.985 | 12.13 | 0.84 |
| 7 | Udder health | 0.43 | 236598 | Fatal | Mas2 | 0.953 | 6.77 | −1.4 |
| 7 | Udder health | 1.147 | 233348 | G Slocum | Mas2 | 0.992 | 12.61 | 1.3 |
| 7 | Udder health | 0.559 | 239278 | Hondo | Mas3 | 0.951 | 8.07 | −0.85 |
| | | | | Aero | | | | |
| 7 | Udder health | 0.61 | 221402 | Chief Mark | Mas3 | 0.969 | 8.99 | −1.2 |
| 7 | Udder health | 0.746 | 226804 | Southwind | Mas3 | 0.982 | 8.66 | 0.92 |
| 7 | Udder health | 0.602 | 236947 | Esentation | UHI | 0.938 | 7.34 | −2.3 |
| 7 | Udder health | 0.746 | 226804 | Southwind | UHI | 0.938 | 7.11 | −1.4 |
| 7 | Udder health | 0.982 | 230104 | T Burma | UHI | 0.955 | 7.63 | 2.4 |
| 7 | Udder health | 1.047 | 237017 | Lord Lily | UHI | 0.947 | 6.56 | 1.8 |

*(1 − [p-value]) = chromosome wide significance level
UHI = Udder health index

### Example 4

BTA15

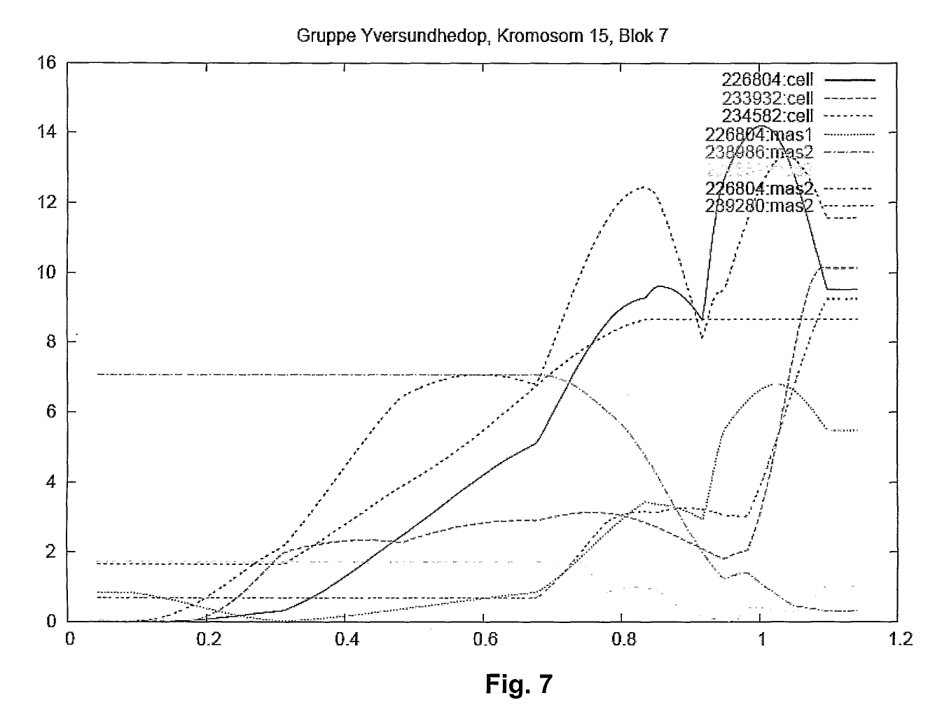

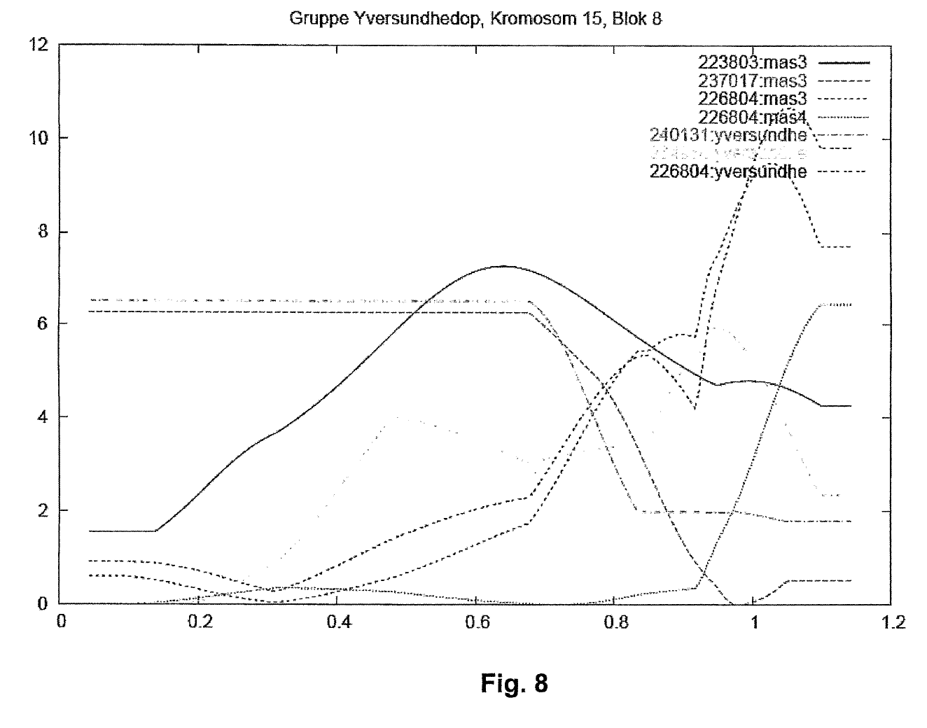

In table 20 the results from the regression analysis for BTA15 are presented. FIG. 7 and FIG. 8 present the QTL graphs for the regression analysis. The variance component method was used to detect QTL across families (including all the sire families in one analysis) for CELL, MAS1, MAS2, MAS3, MAS4, and udder health index in a single trait analysis. The variance component method was used to detect QTL across families (including all the sire families in one analysis) for CELL, MAS1, MAS2, MAS3, MAS4, and udder health index in a single trait analysis. There was no significant QTL detected for CELL, MAS1, MAS2, MAS3, MAS4, and udder health index in the across family analysis. From the multi-trait analysis there is no sign for pleiotrophic QTL affecting the traits CELL, MAS1, MAS2, MAS3, MAS4, and udder health index.

TABLE 20
Significant QTL from the within family analysis using the regression analysis on BTA15

| | | Position | Herdbook | | | p- | | |
|---|---|---|---|---|---|---|---|---|
| Chr | Main trait | (Morgan) | number | Name Sire | Subtrait | value* | F-value | effect |
| 15 | Udder health | 0.836 | 226804 | Southwind | Cell | 0.999 | 14.6 | −0.047 |
| 15 | Udder health | 0.928 | 233932 | Dombinator | Cell | 0.977 | 9.96 | 0.043 |
| 15 | Udder health | 0.948 | 234582 | M Bellwood | Cell | 0.98 | 7.41 | 0.091 |
| 15 | Udder health | 0.852 | 226804 | Southwind | Mas1 | 0.955 | 7.15 | −0.67 |
| 15 | Udder health | 0.692 | 238986 | Mattie G. | Mas2 | 0.976 | 7.71 | −0.86 |
| 15 | Udder health | 0.846 | 239657 | Basar | Mas2 | 0.967 | 8.78 | −1.1 |
| 15 | Udder health | 0.867 | 226804 | Southwind | Mas2 | 0.991 | 11.58 | −1.2 |
| 15 | Udder health | 1.137 | 239280 | Lukas | Mas2 | 0.982 | 8.77 | 1.5 |
| 15 | Udder health | 0.505 | 223803 | B Cleitus | Mas3 | 0.968 | 7.34 | −1.6 |
| 15 | Udder health | 0.675 | 237017 | Lord Lily | Mas3 | 0.977 | 6.48 | −0.7 |
| 15 | Udder health | 0.852 | 226804 | Southwind | Mas3 | 0.991 | 10.64 | −1.1 |
| 15 | Udder health | 0.959 | 226804 | Southwind | Mas4 | 0.947 | 7.02 | −0.99 |
| 15 | Udder health | 0.703 | 240131 | Boudewin | UHI | 0.992 | 9.49 | −2.3 |
| 15 | Udder health | 0.78 | 234984 | Esquimau | UHI | 0.947 | 7.11 | −1.7 |
| 15 | Udder health | 0.882 | 226804 | Southwind | UHI | 0.99 | 10.76 | 1.9 |

*(1 − [p-value]) = chromosome wide significance level
UHI = Udder health index

### Example 5

### BTA21

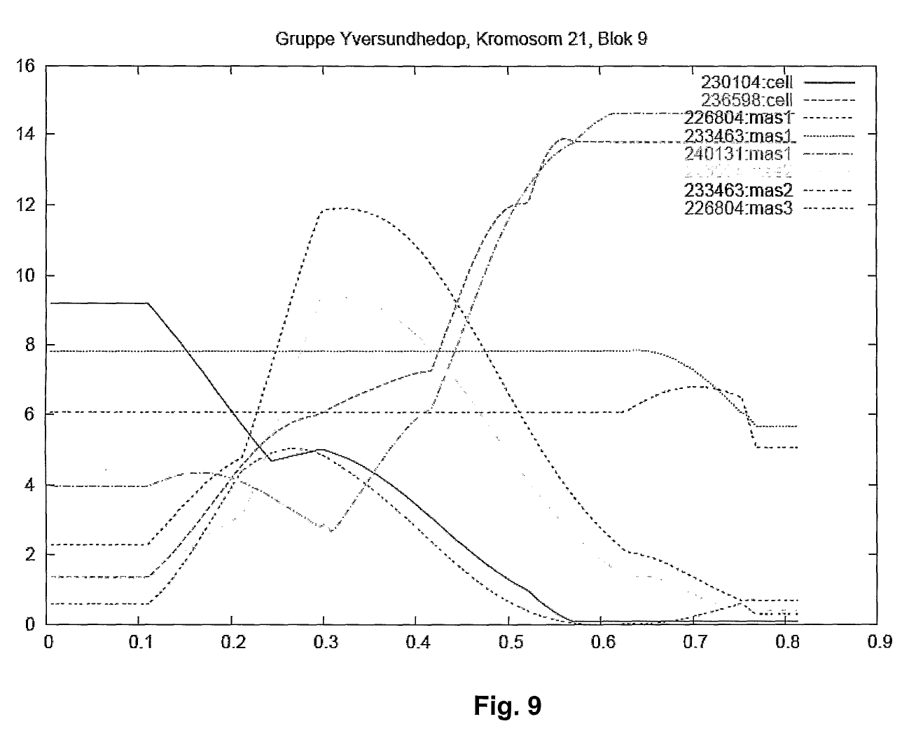

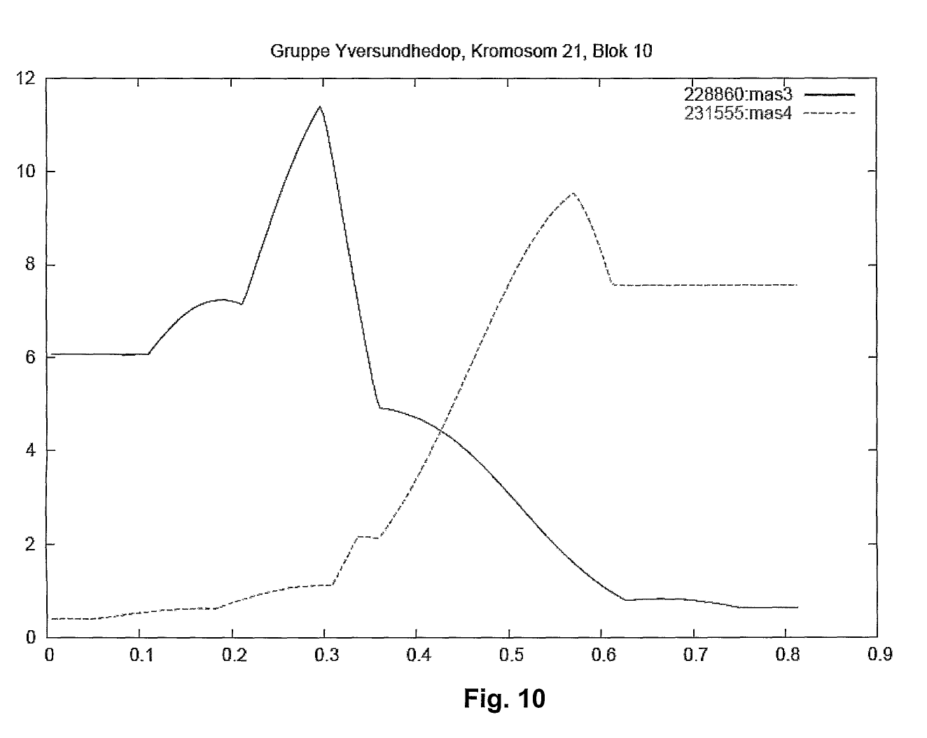

In table 21 the results from the regression analysis for BTA21 are presented. FIG. 9 and FIG. 10 present the QTL graphs for the regression analysis. The variance component method was used to detect QTL across families (including all the sire families in one analysis) for CELL, MAS1, MAS2, MAS3, MAS4, and udder health index in a single trait analysis. There was no significant QTL detected for CELL, MAS1, MAS2, MAS3, MAS4, and udder health index in the across family analysis. From the multi-trait analysis there is no sign for pleiotrophic QTL affecting the traits CELL, MAS1, MAS2, MAS3, MAS4, and udder health index.

TABLE 21
Significant QTL from the within family analysis using the regression analysis on BTA21

| | | Position | Herdbook | | | p- | | |
|---|---|---|---|---|---|---|---|---|
| Chr | Main trait | (Morgan) | number | Name Sire | Subtrait | value* | F-value | effect |
| 21 | Udder health | 0.106 | 230104 | T Burma | Cell | 0.99 | 8.66 | −0.041 |
| 21 | Udder health | 0.563 | 236598 | Fatal | Cell | 0.998 | 13.81 | 0.058 |
| 21 | Udder health | 0.339 | 226804 | Southwind | Mas1 | 0.998 | 12.16 | 0.8 |
| 21 | Udder health | 0.673 | 233463 | E Celsius | Mas1 | 0.992 | 7.74 | −0.63 |
| 21 | Udder health | 0.814 | 240131 | Boudewin | Mas1 | 0.996 | 16.49 | 2.7 |
| 21 | Udder health | 0.326 | 226804 | Southwind | Mas2 | 0.989 | 10.24 | 1.1 |
| 21 | Udder health | 0.738 | 233463 | E Celsius | Mas2 | 0.993 | 9.77 | −1 |
| 21 | Udder health | 0.269 | 226804 | Southwind | Mas3 | 0.924 | 5.55 | 0.77 |
| 21 | Udder health | 0.302 | 228860 | Tesk Holm | Mas3 | 0.991 | 10.75 | −0.73 |
| 21 | Udder health | 0.571 | 231555 | J Jed | Mas4 | 0.985 | 9.56 | 0.88 |

*(1 − [p-value]) = chromosome wide significance level
UHI = Udder health index

### Example 6

### BTA27

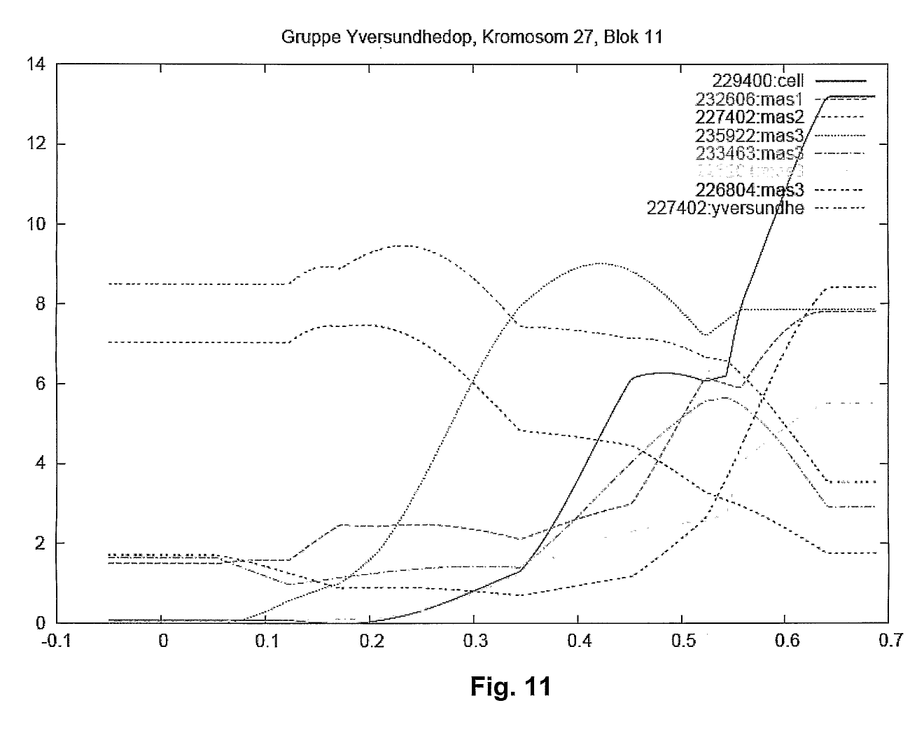

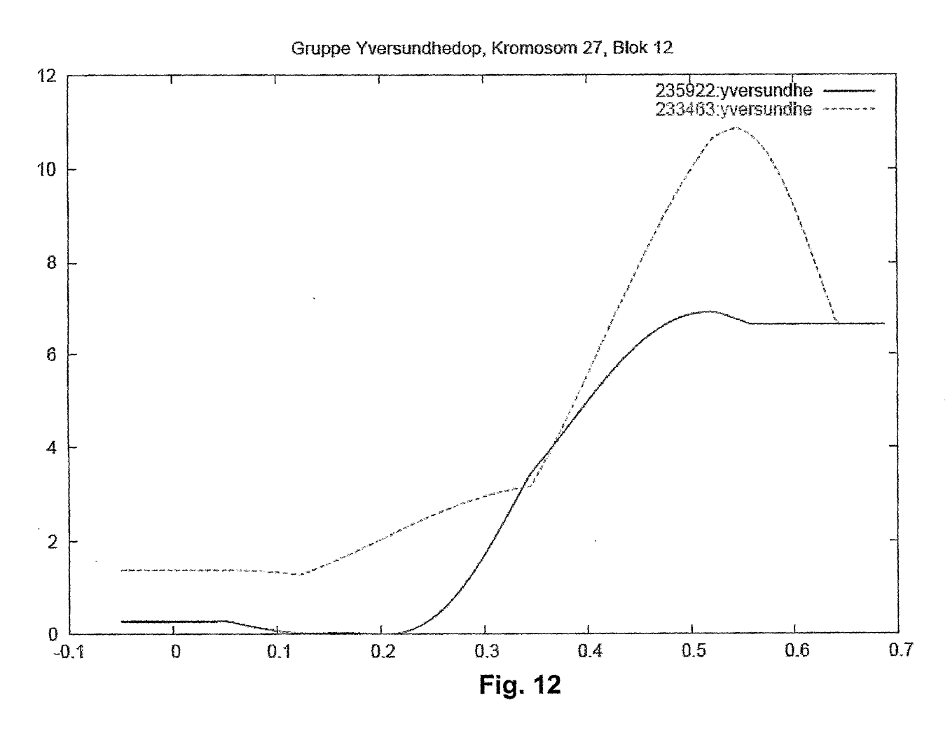

In table 22 the results from the regression analysis for BTA27 are presented. FIG. 11 and FIG. 12 present the QTL graphs for the regression analysis. The variance component method was used to detect QTL across families (including all the sire families in one analysis) for CELL, MAS1, MAS2, MAS3, MAS4, and udder health index in a single trait analysis. A significant QTL was detected in the across family analysis for MAS3 (Likelihood Ratio=6.76, at position 0.60 Morgan). Four sire families contribute to this QTL: 235922, 233463, 226201, and 226804. There was no significant QTL detected for CELL, MAS1, MAS2, MAS4, and udder health index in the across family analysis. From the multi-trait analysis there is no sign for pleiotrophic QTL affecting the traits CELL, MAS1, MAS2, MAS3, MAS4, and udder health index.

TABLE 22
Significant QTL from the within family analysis using the regression analysis on BTA27

| | | Position | Herdbook | | | | | |
|---|---|---|---|---|---|---|---|---|
| Chr | Main trait | (Morgan) | number | Name Sire | Subtrait | p-value* | F-value | effect |
| 27 | Udder health | 0.688 | 229400 | S-B Mascot | Cell | 0.996 | 12.68 | 0.033 |
| 27 | Udder health | 0.64 | 232606 | N Luke | Mas1 | 0.969 | 6.97 | −0.55 |
| 27 | Udder health | 0.2 | 227402 | Aerostar | Mas2 | 0.978 | 7.57 | −0.74 |
| 27 | Udder health | 0.413 | 235922 | East Cash | Mas3 | 0.991 | 10.14 | 1.2 |
| 27 | Udder health | 0.554 | 233463 | E Celsius | Mas3 | 0.943 | 5.85 | 0.68 |
| 27 | Udder health | 0.646 | 226201 | T Blackstar | Mas3 | 0.948 | 6.22 | 0.62 |
| 27 | Udder health | 0.688 | 226804 | Southwind | Mas3 | 0.986 | 8.1 | −0.98 |
| 27 | Udder health | 0.19 | 227402 | Aerostar | UHI | 0.983 | 9.75 | 1.2 |
| 27 | Udder health | 0.512 | 235922 | East Cash | UHI | 0.989 | 10.49 | −1.7 |
| 27 | Udder health | 0.554 | 233463 | E Celsius | UHI | 0.996 | 11.65 | −1.5 |

*(1 − [p-value]) = chromosome wide significance level
UHI = Udder health index

### Example 7

### BTA6

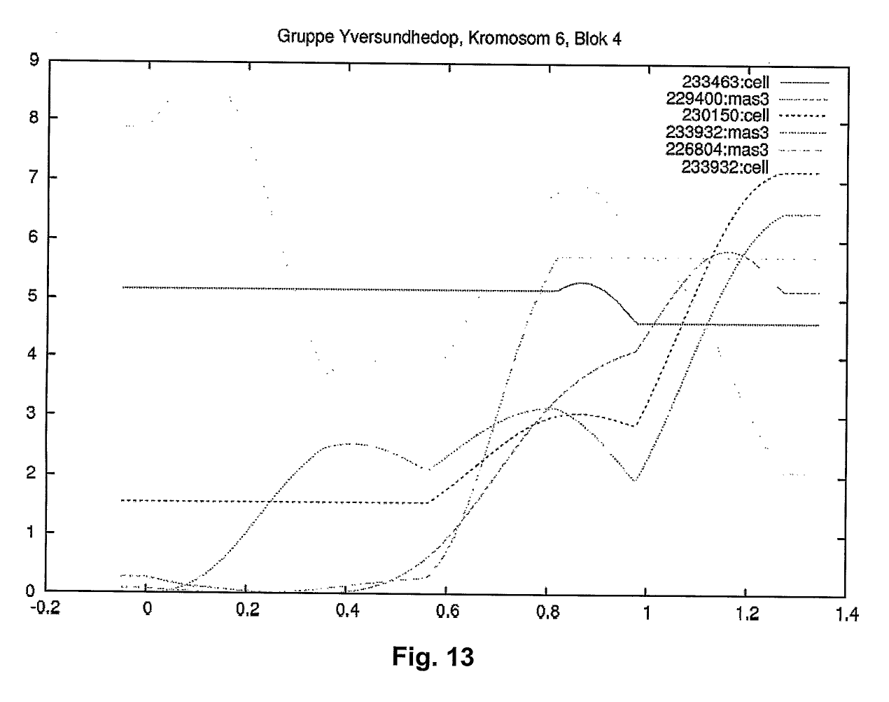

In table 23 the results from the regression analysis for BTA6 are presented. FIG. 13 presents the QTL graphs for the regression analysis. The variance component method was used to detect QTL across families (including all the sire families in one analysis) for CELL, MAS1, MAS2, MAS3, MAS4, and udder health index in a single trait analysis.

TABLE 23
Significant QTL from the within family analysis using the regression analysis on BTA6

| | | Position | Herdbook | | | P- | | |
|---|---|---|---|---|---|---|---|---|
| Chr | Main trait | (Morgan) | number | Name sire | Subtrait | value* | F-value | Effect |
| 6 | Udder health | 0.869 | 233463 | E Celsius | cell | 0.965 | 5.31 | −0.031 |
| 6 | Udder health | 1.294 | 230150 | R Prelude | cell | 0.967 | 7.17 | −0.028 |
| 6 | Udder health | 1.343 | 225602 | R Vanguard | mas1 | 0.951 | 7.35 | −0.91 |
| 6 | Udder health | 0.981 | 229400 | S-B Mascot | mas2 | 0.959 | 7.05 | 0.71 |
| 6 | Udder health | 0.814 | 233463 | E Celsius | mas4 | 0.957 | 4.72 | −0.64 |
| 6 | Udder health | 0.932 | 231900 | B Mountain | mas4 | 0.969 | 5.84 | −0.75 |
| 6 | Udder health | 0.939 | 221402 | Chief Mark | mas4 | 0.966 | 7.79 | −0.87 |

### Example 8

### BTA9

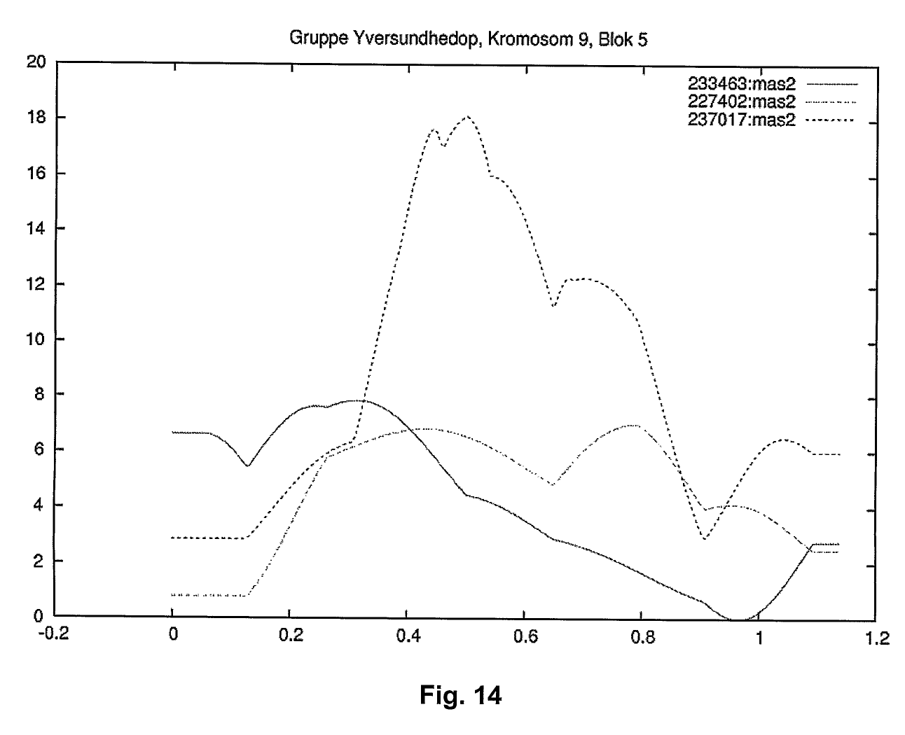

In table 23 the results from the regression analysis for BTA9 are presented. FIG. 14 and

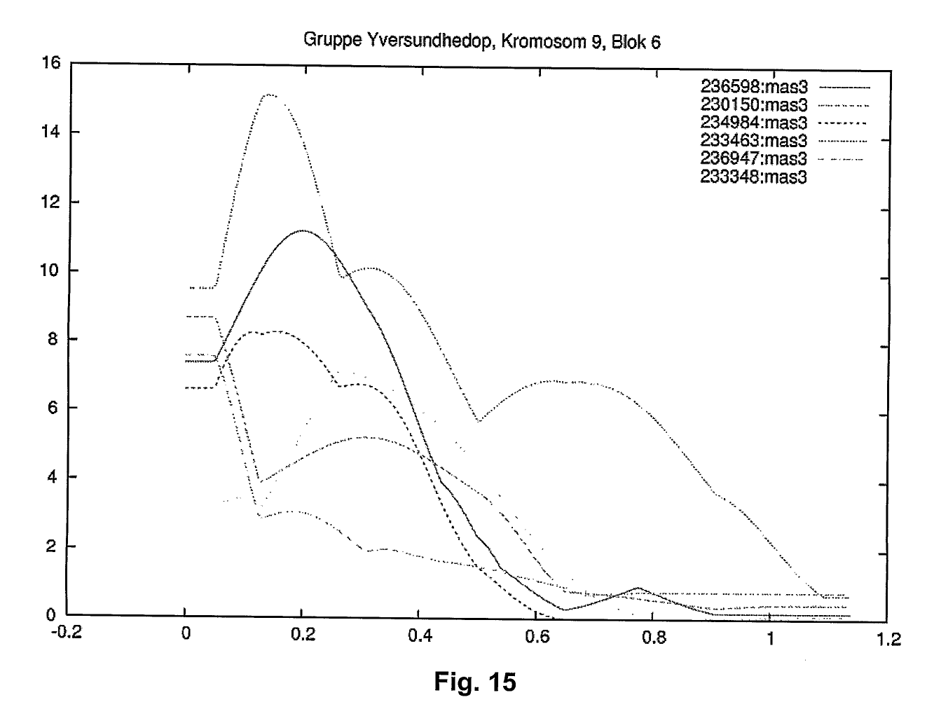

FIG. 15 present the QTL graphs for the regression analysis. The variance component method was used to detect QTL across families (including all the sire families in one analysis) for CELL, MAS1, MAS2, MAS3, MAS4, and udder health index in a single trait analysis.

TABLE 24
Significant QTL from the within family analysis using the regression analysis on BTA9

| | | | Herdbook | | | P- | | |
|---|---|---|---|---|---|---|---|---|
| Chr | Main trait | pos | number | Name sire | Subtrait | value* | F-value | Effect |
| 9 | Udder health | 0.044 | 233463 | E Celsius | cell | 0.968 | 8.26 | −0.036 |
| 9 | Udder health | 0.682 | 236947 | Esentation | mas1 | 0.962 | 7.89 | 1 |
| 9 | Udder health | 0.437 | 237017 | Lord Lily | mas1 | 1 | 18.19 | 0.79 |
| 9 | Udder health | 0.79 | 225602 | R Vanguard | mas1 | 0.984 | 10.14 | −0.85 |
| 9 | Udder health | 0.124 | 238986 | Mattie G. | mas2 | 0.962 | 6.96 | −0.85 |
| 9 | Udder health | 0.5 | 237017 | Lord Lily | mas2 | 1 | 18.13 | 1.3 |
| 9 | Udder health | 0.79 | 227402 | M Aerostar | mas2 | 0.952 | 7.01 | −0.68 |
| 9 | Udder health | 0.312 | 233463 | E Celsius | mas2 | 0.964 | 7.84 | −0.98 |
| 9 | Udder health | 0.044 | 230150 | R Prelude | mas3 | 0.981 | 8.69 | 0.73 |
| 9 | Udder health | 0.044 | 236947 | Esentation | mas3 | 0.952 | 7.57 | −1 |
| 9 | Udder health | 0.136 | 233463 | E Celsius | mas3 | 1 | 15.14 | −1.1 |
| 9 | Udder health | 0.153 | 234984 | Esquimau | mas3 | 0.951 | 8.28 | −0.84 |
| 9 | Udder health | 0.198 | 236598 | Fatal | mas3 | 0.991 | 11.25 | −1.7 |
| 9 | Udder health | 0.266 | 233348 | G Slocum | mas3 | 0.955 | 7.19 | 0.97 |
| 9 | Udder health | 0.124 | 229612 | Belt | mas4 | 0.991 | 13.02 | −1.3 |

### Example 9

### BTA11

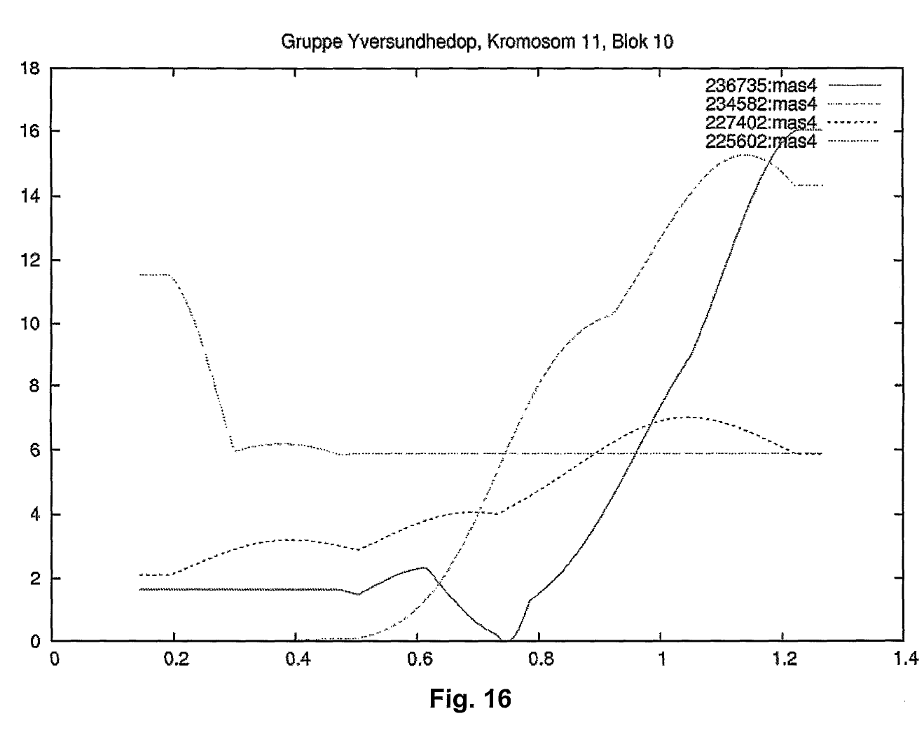

In table 25 the results from the regression analysis for BTA11 are presented. FIG. 16 presents the QTL graphs for the regression analysis. The variance component method was used to detect QTL across families (including all the sire families in one analysis) for CELL, MAS1, MAS2, MAS3, MAS4, and udder health index in a single trait analysis.

TABLE 25
Significant QTL from the within family analysis using the regression analysis on BT11

| | | | Herdbook | | | P- | | |
|---|---|---|---|---|---|---|---|---|
| Chr | Main trait | pos | number | Name sire | Subtrait | value* | F-value | Effect |
| 11 | Udder health | 0.189 | 225602 | R Vanguard | mas4 | 0.996 | 11.55 | −1.4 |
| 11 | Udder health | 1.139 | 234582 | M Bellwood | mas4 | 0.997 | 15.27 | −1.5 |
| 11 | Udder health | 1.049 | 227402 | M Aerostar | mas4 | 0.965 | 7.02 | −0.93 |
| 11 | Udder health | 1.257 | 236735 | Evreux Cle | mas4 | 0.997 | 16.05 | 1.8 |

### Example 10

### BTA26

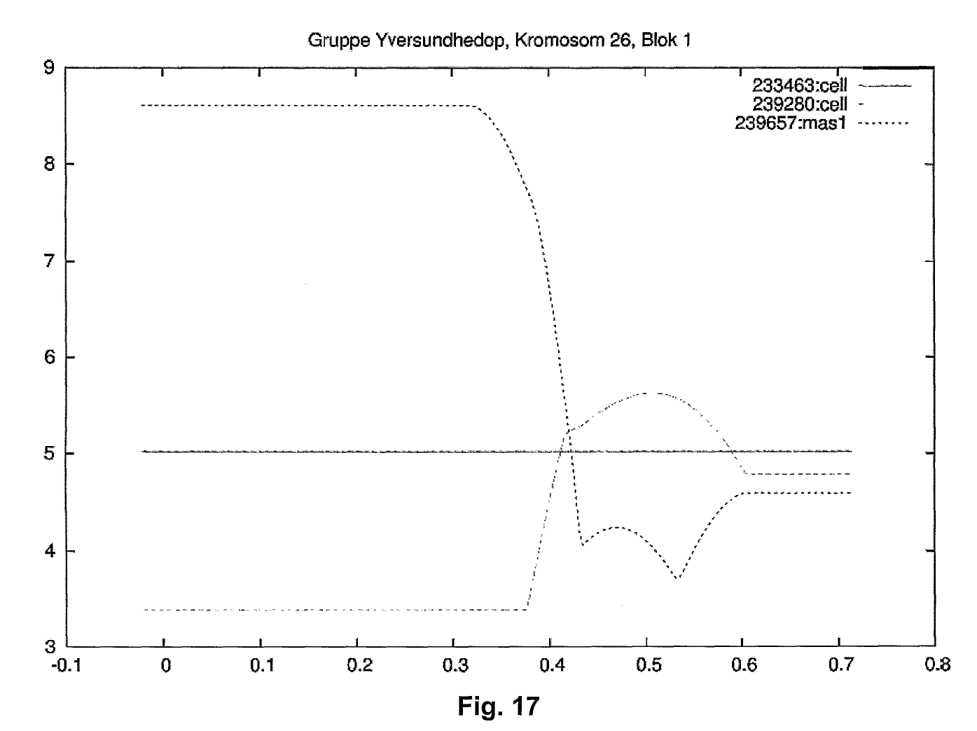

In table 26 the results from the regression analysis for BTA6 are presented. FIGS. 17-19 present the QTL graphs for the regression analysis. The variance component method was used to detect QTL across families (including all the sire families in one analysis) for CELL, MAS1, MAS2, MAS3, MAS4, and udder health index in a single trait analysis.

TABLE 26
Significant QTL from the within family analysis using the regression analysis on BTA26

| | | | Herdbook | | | P- | | |
|---|---|---|---|---|---|---|---|---|
| Chr | Main trait | pos | number | Name sire | Subtrait | value* | F-value | Effect |
| 26 | Udder health | 0.604 | 233463 | E Celsius | cell | 0.977 | 5.02 | 0.029 |
| 26 | Udder health | 0.508 | 239280 | Lukas | cell | 0.959 | 5.62 | 0.048 |
| 26 | Udder health | 0.317 | 239657 | Basar | mas1 | 0.99 | 8.62 | −1 |
| 26 | Udder health | 0.313 | 239657 | Basar | mas2 | 0.986 | 10.28 | −1.3 |
| 26 | Udder health | 0.457 | 231555 | J Jed | mas3 | 0.951 | 5.81 | 0.79 |
| 26 | Udder health | 0.457 | 234347 | Ked Juror | mas3 | 0.991 | 10.95 | −2.5 |
| 26 | Udder health | 0.53 | 233932 | Dombinator | mas3 | 0.991 | 10.89 | 1 |
| 26 | Udder health | 0.534 | 230104 | T Burma | mas3 | 0.95 | 5.06 | 0.81 |
| 26 | Udder health | 0.604 | 233463 | E Celsius | mas3 | 0.956 | 4.27 | −0.64 |
| 26 | Udder health | 0.317 | 237017 | Lord Lily | mas4 | 0.995 | 9.69 | 0.69 |

### Example 11

A QTL study was performed in Danish Holstein Friesian cattle to identify chromosomal regions affecting clinical mastitis in first, second, and third lactations and somatic cell count in first lactation. Significant effects were assessed for associated effects on udder conformation and milk traits. In total eight associations were detected for clinical mastitis on six chromosomes and eight to SCS. Two chromosomes affected both CM and SCS. Four of the QTL affecting clinical mastitis did not have an effect on milk traits and MAS can be performed efficiently for those QTL. Two QTL were found to be linked to QTL affecting milk yield traits and this association must be taken into account in selection.

The example illustrates a study aiming to (1) detect QTL across the cattle genome influencing clinical mastitis, somatic cell score, in Danish Holstein, (2) characterize these QTL for pleiotropy versus multiple linked QTL when chromosomal regions affecting clinical mastitis was also affecting traits in the Danish udder health index or milk production traits. The chromosomes were scanned using a granddaughter design using 19 to 34 grandsire families and 1373 to 2042 sons. A total of 384 microsatellites covering all 29 autosomes were used in the scan. From the across family regression analyses 17 analyses were chromosome wide significant for the primary traits clinical mastitis in first (CM1), second (CM2) and third (CM3) lactations, and somatic cell score in first lactation (SCS). Chromosomes 5, 6, 9, 11, 15, and 26 were found to affect clinical mastitis and chromosomes 5, 6, 8, 13, 22, 23, 24, and 25 affected SCS. Markers on chromosomes 6, 11, 15, and 26 can be used to perform marker assisted selection on clinical mastitis without hampering genetic progress on milk yield, because no effects were realized on the milk traits. Comparing multi-trait models either assuming a pleiotropic QTL affecting two traits or two QTL each affecting one trait, gave some evidence to distinguish between these cases. The most likely models were for BTA5 was a pleiotropic QTL affecting CM2, CM3, and SCS and a linked QTL is affecting fat yield index. For BTA9 the most likely model is a pleiotropic QTL affecting CM1 and CM2 at approximately 8 cM which is linked to a QTL around 58 cM affecting YI.

In Denmark the breeding for improved mastitis resistance is performed by a multi-trait index combining information on treatment for mastitis in 1., 2., and 3. lactations and the correlated indicator traits somatic cell score, dairy form, fore udder attachment, and udder depth. It is of importance to dissect the effect of a given QTL in order to include the QTL information with the proper weight on the different traits in the index.

Mastitis resistance is genetically correlated to milk production traits, which are the economically most important traits. It is therefore essential to investigate if a given QTL that increases the resistance to mastitis also has an effect on the milk production traits. If a chromosomal region is found to affect both traits, it is of importance to know if it is one pleiotropic QTL affecting both traits or if it is linked genes each affecting one trait. In the latter situation it is possible to select for recombinant animals and thereby break a unfavourable correlation due to the linkage.

### Animals

A total genome scan was carried out in the Danish Holstein population. Marker and phenotypic data were collected according to a granddaughter design (Weller et al., 1990). Chromosomes 2, 4, 5, 6, 9, 12, 13, 19, 20, 22, 23, 24, and 25 were analysed in 19 grandsires and 1592 sons, chromosome 17 in 20 families, chromosome BTA14 in 24 grandsirefamilies, chromosome 28 in 33 families and chromosomes 1, 3, 7, 8, 10, 11, 15, 16, 18, 21, 26, 27, and 29 were analysed in 34 grandsires and 2297 sons. Numbers of sons per sire ranged from 20 to 106, with an average family size of 84 for the 19 families and 68 for the 34 families. Sires and their sons were genotyped for marker information whereas phenotypic records were taken from granddaughter performances.

### Markers and Maps

Markers and their positions were chosen from the website of the Meat Animal Research Center: http://www.marc.usda.gov/genome/genome.html. All 29 autosomes were covered in a whole using 384 micro satellite markers with an average marker spacing of 7.97 cM. Markers and positions are given in Buitenhuis et al. 2007 Genotypes were determined on an automated sequence analyser.

### Phenotypic Data

### Primary Traits

The data used were estimated breeding values (EBV) for traits of sons were calculated using a Best Linear Unbiased Prediction (BLUP) model ignoring family structure between sires. Fixed effects in the models were class effects of Herd-year-season, year-month, and calving age (only first parity). The random effects were sire and residuals. For clinical mastitis EBVs were calculated using a single trait model with the risk periods being from 10 days before to 305 days after first calving (CM1), second calving (CM2), and third calving (CM3). Mastitis in each of these periods is recorded as a binary 0/1 trait, where a 1 indicates that the cow was treated for mastitis in the relevant period and a 0 indicates that it was not.

### Secondary Traits

Monthly milkings from first parity were used to calculate the mean somatic cell score in the period 10-180 days after first calving (SCS). Fore udder attachment (UA) and Udder depth (UD) were assessed by classifications on a scale from 1 to 9 in first parity. For milk production traits the official breeding values index were used directly (see http://www.lr.dk/kvaeg/diverse/principles.pdf). For each of the traits milk yield, protein yield, and fat yield a single trait index (MI, PI, and FI) was calculated using a repeatability model over the first three lactations. A function of the three indices define the combined yield index (YI).

### QTL Analysis

A series of analyses were performed. First the data was analysed with a multipoint regression approach for across and within family analysis. If across family chromosome wise significance was obtained for clinical mastitis and at least one more trait, multi trait models were fitted using a variance component method. The models fitted were designed to distinguish if the identified QTL was most likely one QTL affecting both traits (pleiotropy) or two linked QTL each affecting one trait.

### Multi Trait Analysis

For chromosomes affecting two or more traits a multi trait analysis was performed in order to test if the data were better described by a single QTL affecting both traits or by two liked QTL each affecting one trait. Description of those models can be found in Lund et al., 2003.

The pleiotropic and linked-QTL models can be written as:

$$y = X β + Zu + {\sum }_{i = 1}^{nqtl} {Wq}_{i} + e , \qquad ( 1 )$$

where y is a n×t vector of observations on t={1,2} traits, X is a design matrix, 1 is a vector of fixed effects, Z is a matrix relating records to individuals, u is a vector of additive polygenic effects, W is a matrix relating each individual's record to its QTL effect, qi is a vector of additive QTL effects corresponding to the ith QTL, and e is a vector of residuals. The number of QTL, nqtl, is here assumed to be equal to one or two. The random variables u, q, and e are assumed to be multivariate normally distributed and mutually uncorrelated. Specification of pleiotropic and linked QTL models can be seen in Lund et al., 2003. To obtain computational efficiency and stability, the exhaustive search for linked QTL were avoided, by fitting the linked QTL model in maximal likelihood estimates of positions given by single trait VC models. The pleiotropic model were run to cover the region spanning the two positions of the linked QTL model.

Model selection between pleiotropic and linked-QTL models.

The pleiotropic and linked-QTL models can not be compared using likelihood ratio tests because the models are not nested. Therefore, the Bayesian Information Criterion (BIC) (Kass and Raftery 1995; Schwartz 1978) was used to evaluate which model is favoured. The two models entail the same number of parameters and consequently the BIC simplifies to

$$2 log [ \frac{p ( y \vert {\hat{θ}}_{linkage} {M}_{linkage} )}{p ( y \vert {\hat{θ}}_{pleiotropy} {M}_{pleiotropy} )} ] .$$

If the two models are assumed equally likely a priori, the results using this criteria is an approximation to the posterior probability of the pleiotropic model relative to the posterior probability of the linked QTL model. Another less formal criterion used to indicate which model is more likely, is the estimated correlation between QTL effects on the two traits (r_{Q12}) from the pleiotropic model. The rationale behind using r_{Q12} is that if the two traits are under influence of a biallelic pleiotropic QTL the true value of r_{Q12} will be one.

From the across family regression analyses of the primary traits CM1, CM2, CM3, and SCS, 17 results were identified using a 5% chromosome wise significance level across families (Table 27). The affects were found on 13 chromosomes. Eight of the effects were on clinical mastitis. Only two chromosomes reached significance for clinical mastitis in more than one parity. Eight regions were significantly associated with SCS. Two of those were in regions (BTA5 and BTA6) that were also found to affect clinical mastitis, while the remaining six chromosomes gave significant associations to SCS without affecting clinical mastitis.

From the six chromosomes hosting QTL associated with clinical mastitis four of them were significantly associated with correlated traits. BTA5 was associated with SCS and FI. BTA6 with SCS. BTA9 was associated with YI and BTA13 with UD. Finally BTA26 was associated with FI, and YI.

In table 27 P-values for joint chromosome wise tests using a across family regression model for clinical mastitis in first, second, and third lactation (CM1, CM2, and CM3) and somatic cell score (SCS). For chromosomes with significant effects on clinical mastitis significance of QTL affecting udder depth (UD), fore udder attachment (UA), milk yield index (MI), protein yield index (PI), fat yield index (FI), and overall yield index (YI) is indicated.

TABLE 27
p-values for joint chromosome wise tests across families

| | | | | | Correlated |
|---|---|---|---|---|---|
| BTA | CM1 | CM2 | CM3 | SCS | trait |
| BTA5 | | 0.034 | 0.006 | 0.004 | FI |
| BTA6 | | 0.03 | | 0.04 | |
| BTA8 | | | | 0.034 | NA |
| BTA9 | 0.042 | 0.001 | | | YI |
| BTA11 | | | 0.001 | | |
| BTA13 | | | | 0.033 | UA, |
| | | | | | FI, MI |
| BTA15 | 0.036 | | | | |
| BTA22 | | | | 0.001 | UD |
| BTA23 | | | | 0.012 | UD |
| BTA24 | | | | 0.007 | |
| BTA25 | | | | 0.034 | |
| BTA26 | | 0.011 | | | MI, FI, UA, |
| | | | | | UD |

### Pleiotropy Versus Linkage

In situations where a chromosomal region was found to affect clinical mastitis and at least one of the correlated traits it was tested in two-trait models if it was most likely due to one pleiotropic QTL or two linked QTL each affecting one trait. The multitrait models gave some indications to distinguish between linkage and pleiotropy of different QTL (Table 28). The strongest result was on BTA5 where the pleiotropic model for CM2 and CM3 was 1820.5 times more likely than a linked QTL model. On BTA5 two-trait models were run between CM2, CM3, SCS, and FI. The most likely situation is that a pleiotropic QTL is affecting CM2, CM3, and SCS, while a linked QTL is affecting FI. This is in part based on the evidence from Bayes factors, which for all two-trait combinations of CM1, CM2, and SCS show that a pleiotropic model is more likely. The evidence is particularly strong for CM1 and CM2. For models including FI the linkage models were generally more likely. In addition the estimated distance between QTL in the two-trait linkage models we generally higher for combinations including FI (24-46 cM) compared to models between CM1, CM2, and SCS (3.9-14.3).

On BTA6 the correlation between QTL effects on SCS and CM2 from a modeled pleiotropic effect was near unity and in the linkage model the estimates of the two QTL positions were close. Both of which is in concordance with a biallelic pleiotropic QTL, which may therefore be regarded as the most likely situation.

On BTA9 the most likely model is a pleiotropic QTL affecting CM1 and CM2 at approximately 8 cM which is linked to a QTL around 58 cM affecting YI. The second QTL may also affect CM2 but this is less certain. The evidence for pleiotropy of the QTL affecting CM is given in part by limited evidence from the Bayes factors and in part from the fact that the correlation between QTL effects on CM1 and CM2 was unity in the pleiotropic model. The evidence for the QTL for YI is linked from the Bayes factor favors the linkage model as being about 100 times more likely and for both pleiotropic models between YI and CM1 or CM2 the correlations of QTL effects were low at 0.01 and 0.57.

TABLE 28
Results from two trait pleiotropic and linkage models. Correlations
between QTL effects on the two traits in the pleiotropic model,
distance between peaks in a two-QTL linkage model,
and the Bayes factor of a pleiotropic model over a linkage model.

| | | | Distance | |
|---|---|---|---|---|
| Chromosome | Traits | QTL correlation | (cM) | Bayes factor |
| BTA5 | SCS/FI | 0.74 | 30 | 0.07 |
| | SCS/CM2 | 0.69 | 6 | 9.1 |
| | SCS/CM3 | 0.71 | 16 | 4.5 |
| | FI/CM2 | 0.78 | 24 | 1.3 |
| | FI/CM3 | 0.39 | 46 | 0.1 |
| | CM2/CM3 | 0.97 | 22 | 1820.5 |
| BTA6 | SCS/CM2 | 0.99 | 8 | 0.77 |
| BTA9 | CM1/CM2 | 1.0 | 34 | 3.7 |
| | CM1/YI | 0.01 | 14 | 1.0 |
| | CM2/YI | 0.57 | 42 | 0.01 |
| BTA26 | UA/FI | −0.12 | 12 | 1.0 |
| | UA/CM2 | −0.72 | 2 | 10.0 |
| | UD/MI | 0.15 | 8 | 1.0 |
| | FI/CM2 | 0.31 | 14 | 0.77 |
| | FI/MI | 0.46 | 4 | 3.7 |
| | MI/CM2 | NC¹ | 10 | NC |

From the six chromosomes affecting Clinical Mastitis in this example BTA5, BTA6, BTA9, and BTA26 affected highly correlated traits.

Somatic cell score is highly correlated to Clinical Mastitis and to some degree expresses the same response to infections by mastitis pathogens. From the regions affecting Clinical Mastitis, two (BTA5 and BTA6) also affected SCS.

BTA5 affected clinical mastitis in both second and third lactation. Substantial evidence from the Bayes factors allow the distinction between pleiotropy and linkage for BTA5. The most likely situation is that one QTL is affecting CM2, CM3, and SCS and a linked QTL is affecting FI. The phase between the two QTL are such that individuals carrying the positive QTL for Clinical Mastitis generally carry the negative QTL for FI. However, according to our position estimates the two QTL are about 30 cM apart. This is enough to select for recombinant individuals that are positive for the QTL affecting CM as well as the QTL affecting FI. In doing so it should be possible to alter the genetic correlation between the traits to be less antagonistic. BTA5 has been found to be significant for SCS in an overlapping region in North American Holstein Fresians (Heyen et al., 1999).

For BTA6 there was no strong evidence to distinguish pleiotropy from linkage. The small distance between the two positions in the linkage model and the high estimate of the correlation between QTL effects on SCS and CM3 (0.99) indicate that it may be a pleiotropic QTL.

On BTA9 there was little evidence to distinguish linkage from the pleiotropic models. However, the most likely model is a pleiotropic QTL affecting CM1 and CM2 at approximately 8 cM which is linked to a QTL around 58 cM affecting YI. The QTL correlation is strongly antagonistic which means that individuals carrying the positive QTL for Clinical Mastitis generally carry the negative QTL for YI. However, according to our position estimates the two QTL are about 50 cM apart, which is enough to select for recombinant individuals that are positive for the QTL affecting CM as well as the QTL affecting YI. If those individuals are selected they will contribute to a favorable genetic correlation between mastitis and yield. The ability to distinguish between pleiotropic and linkage models is related to the number of informative markers between any linked QTL.

Markers on chromosomes 6, 11, 15, and 26 can be used to perform marker assisted selection on clinical mastitis without hampering genetic progress on milk yield, because no effects were observed on the milk traits. Chromosomes 5 and 9 affected milk yield as well as clinical mastitis, in which case the relationship between the two traits has to be taken into account. In both cases there was some inconclusive evidence that the most likely situation was that linked QTL affecting either mastitis or yield traits were positioned with some distance. If this is the case MAS can be efficient for both traits and even contribute to changing the general genetic correlation between the two traits to be less antagonistic.

In the Nordic system selection is performed to reduce clinical mastitis and SCS is only used as correlated information source. However, SCS is better at measuring subclinical cases which are responsible for a substantial part of the economic losses due to mastitis. Therefore, an economic weight should probably be added also to SCS. If this is the case the QTL on chromosomes 8, 13, 22, 23, 24, and 25 that were only found to affect SCS can be used directly in the selection.

1. A method for determining udder health characteristics in a bovine subject, comprising detecting in a sample from said bovine subject the presence or absence of at least one genetic marker that is linked to at least one trait indicative of udder health,
wherein said at least one genetic marker is located on the bovine chromosome BTA1 in the region flanked by and including the polymorphic microsatellite markers BMS4008 and URB014 and/or
BTA5 in the region flanked by and including the polymorphic microsatellite markers BMS1095 and BM315 and/or
BTA6 in the region flanked by and including the polymorphic microsatellite markers ILSTS093 and BL1038 and/or
BTA7 in the region flanked by and including the polymorphic microsatellite markers BM7160 and BL1043 and/or
BTA9 in the region flanked by and including the polymorphic microsatellite markers BMS2151 and BMS1967 and/or
BTA11 in the region flanked by and including the polymorphic microsatellite markers BM716 and HEL13 and/or
BTA15 in the region flanked by and including the polymorphic microsatellite markers BMS2684 and BMS429 and/or
BTA21 in the region flanked by and including the polymorphic microsatellite markers BMS1117 and BM846 and/or
BTA26 in the region flanked by and including the polymorphic microsatellite markers BMS651 and BM7237 and/or
BTA27 in the region flanked by and including the polymorphic microsatellite markers BMS1001 and BM203,
wherein the presence or absence of said at least one genetic marker is indicative of udder health characteristics of said bovine subject or off-spring therefrom.

2. A method for selecting bovine subjects for breeding purposes, said method comprising by the method in claim 1 determining udder health characteristics.

3. The method according to claim 1, wherein the at least one genetic marker is located in the region of the bovine chromosome BTA1 in the region from about 80.379 to 154.672 cM.

4. The method according to claim 1, wherein the at least one genetic marker is located in the region of the bovine chromosome BTA5 in the region from about 0 to 103.169 cM.

5. The method according to claim 1, wherein the at least one genetic marker is located in the region of the bovine chromosome BTA6 in the region from about 0 to 129.985 cM.

6. The method according to claim 1, wherein the at least one genetic marker is located in the region of the bovine chromosome BTA7 in the region from about 0 to 135.564 cM.

7. The method according to claim 1, wherein the at least one genetic marker is located in the region of the bovine chromosome BTA9 in the region from about 4.892 to 109.287 cM.

8. The method according to claim 1, wherein the at least one genetic marker is located in the region of the bovine chromosome BTA11 in the region from about 19.44 to 122.37 cM.

9. The method according to claim 1, wherein the at least one genetic marker is located in the region of the bovine chromosome BTA15 in the region from about 48.216 to 109.753 cM.

10. The method according to claim 1, wherein the at least one genetic marker is located in the region of the bovine chromosome BTA21 in the region from about 10.969 to 61.247 cM.

11. The method according to claim 1, wherein the at least one genetic marker is located in the region of the bovine chromosome BTA26 in the region from about 2.839 to 66.763 cM.

12. The method according to claim 1, wherein the at least one genetic marker is located in the region of the bovine chromosome BTA27 in the region from about 5.389 to 64.098 cM.

13. The method according to claim 1, wherein the at least one genetic marker is located on the bovine chromosome BTA1 in the region flanked by and including the polymorphic microsatellite markers DIK4151 and BMS1789.

14.-18. (canceled)

19. The method according to claim 1, wherein the at least one genetic marker is located on the bovine chromosome BTA5 in the region flanked by and including the polymorphic microsatellite markers DIK5002 and RM500.

20.-25. (canceled)

26. The method according to claim 1, wherein the at least one genetic marker is located on the bovine chromosome BTA6 in the region flanked by and including the polymorphic microsatellite markers OARJMP36 and BL1038

27.-34. (canceled)

35. The method according to claim 1, wherein the at least one genetic marker is located on the bovine chromosome BTA7 in the region flanked by and including the polymorphic microsatellite markers DIK4606 and BMS2258.

36.-44. (canceled)

45. The method according to claim 1, wherein the at least one genetic marker is located on the bovine chromosome BTA9 in the region flanked by and including the polymorphic microsatellite markers BMS2151 and BMS2819

46.-54. (canceled)

55. The method according to claim 1, wherein the at least one genetic marker is located on the bovine chromosome BTA11 in the region flanked by and including the polymorphic microsatellite markers BMS2047 and HEL13

56.-60. (canceled)

61. The method according to claim 1, wherein the at least one genetic marker is located on the bovine chromosome BTA15 in the region flanked by and including the polymorphic microsatellite markers BMS820 and BMS429.

62.-66. (canceled)

67. The method according to claim 1, wherein the at least one genetic marker is located on the bovine chromosome BTA21 in the region flanked by and including the polymorphic microsatellite markers ILSTS095 and INRA103.

68.-70. (canceled)

71. The method according to claim 1, wherein the at least one genetic marker is located on the bovine chromosome BTA26 in the region flanked by and including the polymorphic microsatellite markers BMS332 and BM7237

72.-80. (canceled)

81. The method according to claim 1, wherein the at least one genetic marker is located on the bovine chromosome BTA27 in the region flanked by and including the polymorphic microsatellite markers INRA134 and BM1857.

82.-88. (canceled)

89. A diagnostic kit for use in detecting the presence or absence in a bovine subject of at least one genetic marker associated with bovine udder health, comprising at least one oligonucleotide sequence selected from the group consisting of SEQ ID NO.: 1 to SEQ ID NO.: 206 and combinations thereof.
